SEQUENCE LISTING (258 sequences)

SEQ ID NO: 1 - protein, 756 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLMSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 2 - protein, 314 aa, Homo sapiens
  SIGNAL (1)..(24)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 3 - protein, 12 aa, Artificial Sequence
  modified IgG4 hinge
ESKYGPPCPPCP

SEQ ID NO: 4 - protein, 41 aa, Homo sapiens
RSKRSRLLHSDYMNMTPRRPGPTRKHYQPYAPPRDFAAYRS

SEQ ID NO: 5 - protein, 112 aa, Artificial Sequence
  mutated human CD3z ITAM-containing activating domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 21 aa, Homo sapiens
LLMIIAPSLGFVLFALFVAFL

SEQ ID NO: 7 - protein, 27 aa, Homo sapiens
FWVLVVVGGVLACYSLLVTVAFIIFWV

SEQ ID NO: 8 - protein, 21 aa, Homo sapiens
IYAGVCISVLVLLALLGVIIA

SEQ ID NO: 9 - protein, 52 aa, Homo sapiens
YFLGRLVPRGRGAAEAATRKQRITETESPYQELQGQRSDVYSDLNTQRPYYK

SEQ ID NO: 10 - protein, 22 aa, Homo sapiens
MLLLVTSLLLCELPHPAFLLIP

SEQ ID NO: 11 - protein, 24 aa, Homo sapiens
MSKEPLILWLMIEFWWLYLTPVTS

SEQ ID NO: 12 - protein, 18 aa, Artificial Sequence
  T2A self-cleaving peptide
EGRGSLLTCGDVEENPGP

SEQ ID NO: 13 - protein, 19 aa, Artificial Sequence
  P2A self-cleaving peptide
ATNFSLLKQAGDVEENPGP

SEQ ID NO: 14 - protein, 20 aa, Artificial Sequence
  E2A self-cleaving peptide
QCTNYALLKLAGDVESNPGP

SEQ ID NO: 15 - protein, 22 aa, Artificial Sequence
  F2A self-cleaving peptide
VKQTLNFDLLKLAGDVESNPGP

SEQ ID NO: 16 - protein, 285 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCH

SEQ ID NO: 17 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 18 - protein, 215 aa, Homo sapiens
MASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 19 - protein, 207 aa, Homo sapiens
MTSRKKVLLKVIILGDSGVGKTSLMNQYVNKKFSNQYKATIGADFLTKEVMVDDRLVTMQIWDTAGQERFQSLGVAFYRGADCCVLVFDVTAPNTFKTLDSWRDEFLIQASPRDPENFPFVVLGNKIDLENRQVATKRAQAWCYSKNNIPYFETSAKEAINVEQAFQTIARNALKQETEVELYNEFPEPIKLDKNDRAKASAESCSC

SEQ ID NO: 20 - protein, 184 aa, Homo sapiens
MREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEKKKPKKKSCLLL

SEQ ID NO: 21 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 22 - protein, 191 aa, Homo sapiens
MQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVLL

SEQ ID NO: 23 - protein, 21 aa, Mus musculus
ILIIACCVGFVLMVLLFLAFL

SEQ ID NO: 24 - protein, 279 aa, Mus musculus
  SIGNAL (1)..(22)
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 25 - protein, 22 aa, Mus musculus
MSKGLLLLWLVTELWWLYLTPA

SEQ ID NO: 26 - protein, 41 aa, Artificial Sequence
  human CD28 costimulatory signaling domain with L186G/L187G substitutions (positions reference to full length protein)
RSKRSRGGHSDYMNMTPRRPGPTRKHYQPYAPPRDFAAYRS

SEQ ID NO: 27 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 23 aa, Artificial Sequence
  T2A self-cleaving peptide variant
LEGGGEGRGSLLTCGDVEENPGP

SEQ ID NO: 29 - protein, 19 aa, Artificial Sequence
  T2A self-cleaving peptide variant
EGRGSLLTCGDVEENPGPR

SEQ ID NO: 30 - protein, 24 aa, Artificial Sequence
  T2A self-cleaving peptide variant
LEGGGEGRGSLLTCGDVEENPGPR

SEQ ID NO: 31 - protein, 27 aa, Artificial Sequence
  P2A self-cleaving peptide variant
RAKRSGSGATNFSLLKQAGDVEENPGP

SEQ ID NO: 32 - protein, 39 aa, Artificial Sequence
  CD28 hinge region
IEVMYPPPYLDNEKSNGTIIHVKGKHLCPSPLFPGPSKP

SEQ ID NO: 33 - protein, 24 aa, Artificial Sequence
  CD8a transmembrane domain
IYIWAPLAGTCGVLLLSLVITLYC

SEQ ID NO: 34 - protein, 111 aa, Homo sapiens
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRAS

SEQ ID NO: 35 - protein, 179 aa, Homo sapiens
TTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 36 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 37 - protein, 295 aa, Homo sapiens
  SIGNAL (1)..(20)
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 38 - protein, 108 aa, Homo sapiens
SVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPK

SEQ ID NO: 39 - protein, 167 aa, Homo sapiens
VTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 40 - protein, 20 aa, Homo sapiens
MHPQVVILSLILHLADSVAG

SEQ ID NO: 41 - protein, 108 aa, Artificial Sequence
  modified Tim1 IgV domain
SVKVGGEAGPSVTLPCHYSGAVTSMCWNRGSCSLFTCQNGIVWTNGTHVTYRKDTRYKLLGDLSRGDVSLTIENTAVSDSGVYCCRVEHRGWFNDMKITVSLEIVPPK

SEQ ID NO: 42 - protein, 290 aa, Homo sapiens [sequence omitted]

SEQ ID NO: 43 - protein, 275 aa, Homo sapiens
SVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 44 - protein, 48 aa, Homo sapiens
KKYFFKKEVQQLSVSFSSLQIKALQNAVEKEVQAEDNIYIENSLYATD

SEQ ID NO: 45 - protein, 43 aa, Homo sapiens
LRGKLMETYCSQKHTRLDYIGDSKNVLNDVQHGREDEDGLFTL

SEQ ID NO: 46 - protein, 274 aa, Artificial Sequence
  TRAF6 signaling domain
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 47 - protein, 193 aa, Artificial Sequence
  TLR8 signaling domain
HHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 48 - protein, 303 aa, Artificial Sequence
  TRAF2 signaling domain
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 49 - protein, 476 aa, Artificial Sequence
  Chimeric Tim - Tim1IgV - Tim1Mucin - Tim1TM - Tim1SD - CD3zSD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 471 aa, Artificial Sequence
  Chimeric Tim - Tim1IgV - Tim1Mucin - Tim1TM - Tim4SD - CD3zSD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIALRGKLMETYCSQKHTRLDYIGDSKNVLNDVQHGREDEDGLFTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 363 aa, Artificial Sequence
  Chimeric Tim - Tim1IgV - Tim1Mucin - CD28TM - CD28SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVRSKRSRLLHSDYMNMTPRRPGPTRKHYQPYAPPRDFAAYRS

SEQ ID NO: 52 - protein, 590 aa, Artificial Sequence
  Chimeric Tim - Tim1IgV - Tim1Mucin -Tim1TM - TRAF6SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 53 - protein, 596 aa, Artificial Sequence
  Chimeric Tim - Tim1IgV - Tim1Mucin -CD28TM - TRAF6SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 54 - protein, 619 aa, Artificial Sequence
  Chimeric Tim - Tim1IgV - Tim1Mucin - Tim1TM - TRAF2SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 55 - protein, 625 aa, Artificial Sequence
  Chimeric Tim - Tim1IgV - Tim1Mucin - CD28TM - TRAF2SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 56 - protein, 621 aa, Artificial Sequence
  Chimeric Tim - Tim1IgV - Tim1Mucin - Tim1TM - TLR8SD - CD3zSD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 415 aa, Artificial Sequence
  Chimeric Tim - Tim1IgV - Tim1Mucin - CD28TM - CD28SD - DAP12SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 58 - protein, 409 aa, Artificial Sequence
  Chimeric Tim - Tim1IgV - Tim1Mucin - Tim1TM - CD28SD - DAP12SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIARSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 59 - protein, 490 aa, Artificial Sequence
  Chimeric Tim - Tim4IgV - Tim4Mucin - Tim4TM - Tim4SD - CD3zSD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 495 aa, Artificial Sequence
  Chimeric Tim - Tim4IgV - Tim4Mucin - Tim4TM - Tim1SD - CD3zSD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 382 aa, Artificial Sequence
  Chimeric Tim - Tim4IgV - Tim4Mucin - CD28TM - CD28SD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRS

SEQ ID NO: 62 - protein, 609 aa, Artificial Sequence
  Chimeric Tim - Tim4IgV - Tim4Mucin - Tim4TM - TRAF6SD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 63 - protein, 615 aa, Artificial Sequence
  Chimeric Tim - Tim4IgV - Tim4Mucin - CD28TM - TRAF6SD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 64 - protein, 638 aa, Artificial Sequence
  Chimeric Tim - Tim4IgV - Tim4Mucin - Tim4TM - TRAF2SD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 65 - protein, 644 aa, Artificial Sequence
  Chimeric Tim - Tim4IgV - Tim4Mucin - CD28TM - TRAF2SD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 66 - protein, 644 aa, Artificial Sequence; identical to SEQ ID NO: 65
  Chimeric Tim - Tim4IgV - Tim4Mucin - Tim1TM - TLR8SD - CD3zSD

SEQ ID NO: 67 - protein, 628 aa, Artificial Sequence
  Chimeric Tim - Tim4IgV - Tim4Mucin - Tim1TM - TLR8SD - CD3zSD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 416 aa, Artificial Sequence
  Chimeric Tim - Tim4IgV - Tim1Mucin - Tim1TM - CD28SD - DAP12SD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIARSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 69 - protein, 422 aa, Artificial Sequence
  Chimeric Tim - Tim4IgV - Tim1Mucin - CD28TM - CD28SD - DAP12SD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 70 - protein, 45 aa, Artificial Sequence
  CD8a hinge region
TTTPAPRPPTPAPTIASQPLSLRPEACRPAAGGAVHTRGLDFACD

SEQ ID NO: 71 - protein, 496 aa, Artificial Sequence
  Chimeric Tim - TimIgV - Tim4Mucin - CD28TM - CD28SD - CD3zSD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 72 - protein, 510 aa, Artificial Sequence
  hTIM4-TIM4-TIM4tm-TLR2ICD
  SIGNAL (1)..(24)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 73 - protein, 528 aa, artificial sequence
  hTIM4-TIM4-TIM4tm-TLR8ICD
  SIGNAL (1)..(24)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 74-88 - skipped (15 entries)

SEQ ID NO: 89 - protein, 15 aa, Artificial Sequence
  Flexible linker
GGGGSGGGGSGGGGS

SEQ ID NO: 90 - protein, 15 aa, Artificial Sequence
  Flexible linker
GTGGSGGTGSGTGTS

SEQ ID NO: 91 - protein, 12 aa, Artificial Sequence
  IgG4 hinge region (short)
ESKYGPPCPPCP

SEQ ID NO: 92 - protein, 45 aa, Artificial Sequence; identical to SEQ ID NO: 70
  CD8a hinge region

SEQ ID NO: 93 - protein, 39 aa, Artificial Sequence; identical to SEQ ID NO: 32
  CD28 hinge region

SEQ ID NO: 94 - protein, 24 aa, Artificial Sequence
  CD8a transmembrane region
IYIWAPLAGTCGVLLLSLVITLYC

SEQ ID NO: 95 - protein, 27 aa, Artificial Sequence
  CD28 transmembrane region
FWVLVVVGGVLACYSLLVTVAFIIFWV

SEQ ID NO: 96 - protein, 113 aa, Artificial Sequence; identical to SEQ ID NO: 27
  wildtype CD3z signaling domain

SEQ ID NO: 97 - protein, 112 aa, Artificial Sequence; identical to SEQ ID NO: 5
  variant CD3z signaling domain

SEQ ID NO: 98 - protein, 41 aa, Artificial Sequence; identical to SEQ ID NO: 4
  wildtype CD28 costimulatory signaling domain

SEQ ID NO: 99 - protein, 41 aa, Artificial Sequence; identical to SEQ ID NO: 26
  variant CD28 costimulatory signaling domain with L186G/L187G substitutions with positions in reference to full length protein

SEQ ID NO: 100 - protein, 42 aa, Artificial Sequence
  4-1BB costimulatory signaling domain
KRGRKKLLYIFKQPFMRPVQTTQEEDGCSCRFPEEEEGGCEL

SEQ ID NO: 101-116 - skipped (16 entries)

SEQ ID NO: 117 - protein, 21 aa, Artificial Sequence
  GMCSFR_Signal Peptide
MLLVTSLLLCELPHPAFLLIP

SEQ ID NO: 118 - protein, 41 aa, Homo sapiens; identical to SEQ ID NO: 4

SEQ ID NO: 119 - protein, 41 aa, Artificial Sequence; identical to SEQ ID NO: 26
  CD28 costimulatory signaling domain with L186G/L187G substitutions (positions reference to full length protein)

SEQ ID NO: 120 - protein, 42 aa, Artificial Sequence
  OX40 costimulatory signaling domain
ALYLLRRDQRLPPDAHKPPGGGSFRTPIQEEQADAHSTLAKI

SEQ ID NO: 121 - protein, 116 aa, Artificial Sequence
  CD2 costimulatory signaling domain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 122 - protein, 42 aa, Artificial Sequence; identical to SEQ ID NO: 100
  4-1BB costimulatory signaling domain

SEQ ID NO: 123 - protein, 48 aa, Artificial Sequence
  CD27 costimulatory signaling domain
QRRKYRSNKGESPVEPAEPCHYSCPREEEGSTIPIQEDYRKPEPACSP

SEQ ID NO: 124 - protein, 29 aa, Artificial Sequence
  ICAM-1 costimulatory signaling domain
NRQRKIKKYRLQQAQKGTPMKPNTQATPP

SEQ ID NO: 125 - protein, 46 aa, Artificial Sequence
  LFA-1 costimulatory signaling domain
KALIHLSDLREYRRFEKEKLKSQWNNDNPLFKSATTTVMNPKFAES

SEQ ID NO: 126 - protein, 38 aa, Artificial Sequence
  ICOS costimulatory signaling domain
CWLTKKKYSSSVHDPNGEYMFMRAVNTAKKSRLTDVTL

SEQ ID NO: 127 - protein, 188 aa, Artificial Sequence
  CD30 costimulatory signaling domain
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 128 - protein, 62 aa, Artificial Sequence
  CD40 costimulatory signaling domain
KKVAKKPTNKAPHPKQEPQEINFPDDLPGSNTAAPVQETLHGCQPVTQEDGKESRISVQERQ

SEQ ID NO: 129 - protein, 97 aa, Artificial Sequence
  PD-1 costimulatory signaling domain
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 130 - protein, 39 aa, Artificial Sequence
  CD7 costimulatory signaling domain
RTQIKKLCSWRDKNSAACVVYEDMSHSRCNTLSSPNQYQ

SEQ ID NO: 131 - protein, 37 aa, Artificial Sequence
  LIGHT costimulatory signaling domain
MEESVVRPSVFVVDGQTDIPFTRLGRSHRRQSCSVAR

SEQ ID NO: 132 - protein, 70 aa, Artificial Sequence
  NKG2C costimulatory signaling domain
MSKQRGTFSEVSLAQDPKRQQRKPKGNKSSISGTEQEIFQVELNLQNPSLNHQGIDKIYDCQGLLPPPEK

SEQ ID NO: 133 - protein, 47 aa, Artificial Sequence
  B7-H3 costimulatory signaling domain
CWRKIKQSCEEENAGAEDQDGEGEGSKTALQPLKHSDSKEDDGQEIA

SEQ ID NO: 134 - protein, 113 aa, Homo sapiens; identical to SEQ ID NO: 27

SEQ ID NO: 135 - protein, 112 aa, Artificial Sequence; identical to SEQ ID NO: 5
  mutated human CD3z ITAM-containing activating domain

SEQ ID NO: 136 - protein, 45 aa, Artificial Sequence
  CD3g ITAM-containing activating domain
GQDGVRQSRASDKQTLLPNDQLYQPLKDREDDQYSHLQGNQLRRN

SEQ ID NO: 137 - protein, 45 aa, Artificial Sequence
  CD3d ITAM-containing activating domain
GHETGRLSGAADTQALLRNDQVYQPLRDRDDAQYSHLGGNWARNK

SEQ ID NO: 138 - protein, 55 aa, Artificial Sequence
  CD3e ITAM-containing activating domain
KNRKAKAKPVTRGAGAGGRQRGQNKERPPPVPNPDYEPIRKGQRDLYSGLNQRRI

SEQ ID NO: 139 - protein, 93 aa, Artificial Sequence
  CD5 ITAM-containing activating domain
KKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 140 - protein, 141 aa, Artificial Sequence
  CD22 ITAM-containing activating domain
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 141 - protein, 61 aa, Artificial Sequence
  CD79a ITAM-containing activating domain
RKRWQNEKLGLDAGDEYEDENLYEGLNLDDCSMYEDISRGLQGTYQDVGSLNIGDVQLEKP

SEQ ID NO: 142 - protein, 24 aa, Artificial Sequence
  DAP10 ITAM-containing activating domain
LCARPRRSPAQEDGKVYINMPGRG

SEQ ID NO: 143 - protein, 76 aa, Artificial Sequence
  CD66d ITAM-containing activating domain
AKTGRTSIQRDLKEQQPQALAPGRGPSHSSAFSMSPLSTAQAPLPNPRTAASIYEELLKHDTNIYCRMDHKAEVAS

SEQ ID NO: 144 - protein, 21 aa, Artificial Sequence
  Tim4 transmembrane domain
LLMIIAPSLGFVLFALFVAFL

SEQ ID NO: 145 - protein, 27 aa, Artificial Sequence
  CD28 transmembrane domain
FWVLVVVGGVLACYSLLVTVAFIIFWV

SEQ ID NO: 146 - protein, 27 aa, Artificial Sequence
  4-1BB transmembrane domain
IISFFLALTSTALLFLLFFLTLRFSVV

SEQ ID NO: 147 - protein, 21 aa, Artificial Sequence
  OX40 transmembrane domain
VAAILGLGLVLGLLGPLAILL

SEQ ID NO: 148 - protein, 21 aa, Artificial Sequence
  CD27 transmembrane domain
ILVIFSGMFLVFTLAGALFLH

SEQ ID NO: 149 - protein, 21 aa, Artificial Sequence
  ICOS transmembrane domain
FWLPIGCAAFVVVCILGCILI

SEQ ID NO: 150 - protein, 26 aa, Artificial Sequence
  CD2 transmembrane domain
IYLIIGICGGGSLLMVFVALLVFYIT

SEQ ID NO: 151 - protein, 23 aa, Artificial Sequence
  LFA-1 transmembrane domain
IAAIVGGTVAGIVLIGILLLVIW

SEQ ID NO: 152 - protein, 28 aa, Artificial Sequence
  CD30 transmembrane domain
PVLDAGPVLFWVILVLVVVVGSSAFLLC

SEQ ID NO: 153 - protein, 22 aa, Artificial Sequence
  CD40 transmembrane domain
ALVVIPIIFGILFAILLVLVFI

SEQ ID NO: 154 - protein, 21 aa, Artificial Sequence
  PD-1 transmembrane domain
VGVVGGLLGSLVLLVWVLAVI

SEQ ID NO: 155 - protein, 21 aa, Artificial Sequence
  CD7 transmembrane domain
AALAVISFLLGLGLGVACVLA

SEQ ID NO: 156 - protein, 21 aa, Artificial Sequence
  LIGHT transmembrane domain
VGLGLLLLLMGAGLAVQGWFL

SEQ ID NO: 157 - protein, 23 aa, Artificial Sequence
  NKG2C transmembrane domain
LTAEVLGIICIVLMATVLKTIVL

SEQ ID NO: 158 - protein, 21 aa, Artificial Sequence
  B7-H3 transmembrane domain
LWVTVGLSVCLIALLVALAFV

SEQ ID NO: 159 - protein, 347 aa, Artificial Sequence
  Tim4 binding domain-CD28 transmembrane-CD28 costimulatory signaling domain
  SIGNAL (1)..(22)
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFWVLVVVGGVLACYSLLVTVAFIIFWVRSKRSRGGHSDYMNMTPRRPGPTRKHYQPYAPPRDFAAYRS

SEQ ID NO: 160 - protein, 341 aa, Artificial Sequence
  Tim4 binding domain-Tim4 transmembrane-CD28 costimulatory signaling domain
  SIGNAL (1)..(22)
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLRSKRSRGGHSDYMNMTPRRPGPTRKHYQPYAPPRDFAAYRS

SEQ ID NO: 161 - protein, 454 aa, Artificial Sequence
  Tim4 binding domain-Tim4 transmembrane-4-1BB costimulatory signaling domain-CD3z ITAM containing activating domain
  SIGNAL (1)..(22)
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 496 aa, Artificial Sequence; identical to SEQ ID NO: 71
  Construct 13ATIM4 binding domain - CD28 TM - CD28 costim - CD3z
  SIGNAL (1)..(24)

SEQ ID NO: 163 - protein, 22 aa, Artificial Sequence
  GM-CSF signal peptide
MLLLVTSLLLCELPHPAFLLIP

SEQ ID NO: 164 - protein, 41 aa, Homo sapiens; identical to SEQ ID NO: 4

SEQ ID NO: 165 - protein, 41 aa, Artificial Sequence; identical to SEQ ID NO: 26
  CD28 costimulatory signaling domain with L186G/L187G substitutions (positions reference to full length protein)

SEQ ID NO: 166 - protein, 42 aa, Artificial Sequence; identical to SEQ ID NO: 120
  OX40 costimulatory signaling domain

SEQ ID NO: 167 - protein, 116 aa, Artificial Sequence; identical to SEQ ID NO: 121
  CD2 costimulatory signaling domain

SEQ ID NO: 168 - protein, 42 aa, Artificial Sequence; identical to SEQ ID NO: 100
  4-1BB costimulatory signaling domain

SEQ ID NO: 169 - protein, 48 aa, Artificial Sequence; identical to SEQ ID NO: 123
  CD27 costimulatory signaling domain

SEQ ID NO: 170 - protein, 29 aa, Artificial Sequence
  ICAM-1 costimulatory signaling domain
NRQRKIKKYRLQQAQKGTPMKPNTQATPP

SEQ ID NO: 171 - protein, 46 aa, Artificial Sequence; identical to SEQ ID NO: 125
  LFA-1 costimulatory signaling domain

SEQ ID NO: 172 - protein, 38 aa, Artificial Sequence; identical to SEQ ID NO: 126
  ICOS costimulatory signaling domain

SEQ ID NO: 173 - protein, 188 aa, Artificial Sequence; identical to SEQ ID NO: 127
  CD30 costimulatory signaling domain

SEQ ID NO: 174 - protein, 62 aa, Artificial Sequence; identical to SEQ ID NO: 128
  CD40 costimulatory signaling domain

SEQ ID NO: 175 - protein, 97 aa, Artificial Sequence; identical to SEQ ID NO: 129
  PD-1 costimulatory signaling domain

SEQ ID NO: 176 - protein, 39 aa, Artificial Sequence; identical to SEQ ID NO: 130
  CD7 costimulatory signaling domain

SEQ ID NO: 177 - protein, 37 aa, Artificial Sequence; identical to SEQ ID NO: 131
  LIGHT costimulatory signaling domain

SEQ ID NO: 178 - protein, 70 aa, Artificial Sequence; identical to SEQ ID NO: 132
  NKG2C costimulatory signaling domain

SEQ ID NO: 179 - protein, 47 aa, Artificial Sequence; identical to SEQ ID NO: 133
  B7-H3 costimulatory signaling domain

SEQ ID NO: 180 - protein, 52 aa, Artificial Sequence; identical to SEQ ID NO: 9
  DAP12

SEQ ID NO: 181 - protein, 27 aa, Artificial Sequence
  CD28 transmembrane domain
FWVLVVVGGVLACYSLLVTVAFIIFWV

SEQ ID NO: 182 - protein, 27 aa, Artificial Sequence
  4-1BB transmembrane domain
IISFFLALTSTALLFLLFFLTLRFSVV

SEQ ID NO: 183 - protein, 21 aa, Artificial Sequence
  OX40 transmembrane domain
VAAILGLGLVLGLLGPLAILL

SEQ ID NO: 184 - protein, 21 aa, Artificial Sequence
  CD27 transmembrane domain
ILVIFSGMFLVFTLAGALFLH

SEQ ID NO: 185 - protein, 21 aa, Artificial Sequence
  ICOS transmembrane domain
FWLPIGCAAFVVVCILGCILI

SEQ ID NO: 186 - protein, 26 aa, Artificial Sequence
  CD2 transmembrane domain
IYLIIGICGGGSLLMVFVALLVFYIT

SEQ ID NO: 187 - protein, 23 aa, Artificial Sequence
  LFA-1 transmembrane domain
IAAIVGGTVAGIVLIGILLLVIW

SEQ ID NO: 188 - protein, 28 aa, Artificial Sequence
  CD30 transmembrane domain
PVLDAGPVLFWVILVLVVVVGSSAFLLC

SEQ ID NO: 189 - protein, 22 aa, Artificial Sequence
  CD40 transmembrane domain
ALVVIPIIFGILFAILLVLVFI

SEQ ID NO: 190 - protein, 21 aa, Artificial Sequence
  PD-1 transmembrane domain
VGVVGGLLGSLVLLVWVLAVI

SEQ ID NO: 191 - protein, 21 aa, Artificial Sequence
  CD7 transmembrane domain
AALAVISFLLGLGLGVACVLA

SEQ ID NO: 192 - protein, 21 aa, Artificial Sequence
  LIGHT transmembrane domain
VGLGLLLLLMGAGLAVQGWFL

SEQ ID NO: 193 - protein, 23 aa, Artificial Sequence
  NKG2C transmembrane domain
LTAEVLGIICIVLMATVLKTIVL

SEQ ID NO: 194 - protein, 21 aa, Artificial Sequence
  B7-H3 transmembrane domain
LWVTVGLSVCLIALLVALAFV

SEQ ID NO: 195 - protein, 473 aa, Artificial Sequence
  Tim4 binding domain-CD28 hinge-CD28 transmembrane-CD28 costimulatory signaling domain-DAP12 signaling domain, amino acids 1-24 are signal peptide(CTX140)
  SIGNAL (1)..(24)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 196 - protein, 446 aa, Artificial Sequence
  Tim4 binding domain-modified IgG4 hinge-CD28 transmembrane-CD28 costimulatory signaling domain-DAP12 signaling domain, amino acids 1-24 are signal peptide(CTX143)
  SIGNAL (1)..(24)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQESKYGPPCPPCPFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 197 - protein, 434 aa, Artificial Sequence
  Tim4 binding domain-CD28 transmembrane-CD28 costimulatory signaling domain-DAP12 signaling domain, amino acids 1-24 are signal peptide (CTX137)
  SIGNAL (1)..(24)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 198 - protein, 428 aa, Artificial Sequence
  Tim4 binding domain - Tim4 TM - CD28 - DAP12; amino acids 1-24 are signal peptide(CTX256)
  SIGNAL (1)..(24)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 199-217 - skipped (19 entries)

SEQ ID NO: 218 - protein, 24 aa, Homo sapiens
MSKEPLILWLMIEFWWLYLTPVTS

SEQ ID NO: 219 - protein, 290 aa, Homo sapiens [sequence omitted]

SEQ ID NO: 220 - protein, 27 aa, Homo sapiens
FWVLVVVGGVLACYSLLVTVAFIIFWV

SEQ ID NO: 221 - protein, 21 aa, Homo sapiens
LLMIIAPSLGFVLFALFVAFL

SEQ ID NO: 222 - protein, 175 aa, Homo sapiens
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 223 - protein, 193 aa, Homo sapiens; identical to SEQ ID NO: 47

SEQ ID NO: 224 - protein, 42 aa, Homo sapiens
RGKLMETYCSQKHTRLDYIGDSKNVLNDVQHGREDEDGLFTL

SEQ ID NO: 225 - protein, 43 aa, Homo sapiens
MRGKLMETYCSQKHTRLDYIGDSKNVLNDVQHGREDEDGLFTL

SEQ ID NO: 226 - skipped

SEQ ID NO: 227 - protein, 496 aa, Artificial Sequence
  hTIM4-TIM4-CD28tm-CD28icd-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 228 - protein, 671 aa, Artificial Sequence
  hTIM4-TIM4-CD28tm-CD28icd-TLR2ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRSKRSRLLHSDYMNMTPRRPGPTRKHYQPYAPPRDFAAYRSHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 229 - protein, 671 aa, Artificial Sequence
  hTIM4-TIM4-CD28tm-CD28icd-CD3zICD-TLR2ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 230 - protein, 689 aa, Artificial Sequence
  hTIM4-TIM4-CD28tm-CD28icd-TLR8ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRSKRSRLLHSDYMNMTPRRPGPTRKHYQPYAPPRDFAAYRSHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 231 - protein, 689 aa, Artificial Sequence
  hTIM4-TIM4-CD28tm-CD28icd-CD3zICD-TLR8ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 232 - protein, 630 aa, Artificial Sequence
  hTIM4-TIM4-CD28tm-TLR2ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 233 - protein, 630 aa, Artificial Sequence
  hTIM4-TIM4-CD28tm-CD3zICD-TLR2ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 234 - protein, 648 aa, Artificial Sequence
  hTIM4-TIM4-CD28tm-TLR8ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 235 - protein, 648 aa, Artificial Sequence
  hTIM4-TIM4-CD28tm-CD3zICD-TLR8ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 236 - protein, 729 aa, Artificial Sequence
  hTIM4-TIM4-CD28tm-TRAF6ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 237 - protein, 729 aa, Artificial Sequence
  hTIM4-TIM4-CD28tm-CD3zICD-TRAF6ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQQFWVLVVVGGVLACYSLLVTVAFIIFWVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 238 - protein, 490 aa, Artificial Sequence
  hTIM4-TIM4-TIM4tm-CD28icd-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 239 - protein, 665 aa, Artificial Sequence
  hTIM4-TIM4-TIM4tm-CD28icd-TLR2ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRSKRSRLLHSDYMNMTPRRPGPTRKHYQPYAPPRDFAAYRSHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 240 - protein, 665 aa, Artificial Sequence
  hTIM4-TIM4-TIM4tm-CD28icd-CD3zICD-TLR2ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 241 - protein, 683 aa, Artificial Sequence
  hTIM4-TIM4-TIM4tm-CD28icd-TLR8ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRSKRSRLLHSDYMNMTPRRPGPTRKHYQPYAPPRDFAAYRSHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 242 - protein, 683 aa, Artificial Sequence
  hTIM4-TIM4-TIM4tm-CD28icd-CD3zICD-TLR8ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 243 - protein, 624 aa, Artificial Sequence
  hTIM4-TIM4-TIM4tm-TLR2ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 244 - protein, 624 aa, Artificial Sequence
  hTIM4-TIM4-TIM4tm-CD3zICD-TLR2ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 245 - protein, 642 aa, Artificial Sequence
  hTIM4-TIM4-TIM4tm-TLR8ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 246 - protein, 642 aa, Artificial Sequence
  hTIM4-TIM4-TIM4tm-CD3zICD-TLR8ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 247 - protein, 723 aa, Artificial Sequence
  hTIM4-TIM4-TIM4tm-TRAF6ICD-CD3zICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 248 - protein, 723 aa, Artificial Sequence
  hTIM4-TIM4-TIM4tm-CD3zICD-TRAF6ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 249 - protein, 379 aa, Artificial Sequence
  hTIM4-TIM4-TIM4tm-TIM4ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLMRGKLMETYCSQKHTRLDYIGDSKNVLNDVQHGREDEDGLFTL

SEQ ID NO: 250 - protein, 666 aa, Artificial Sequence
  hTIM4-TIM4-TIM4tm-TIM4ICD-TLR2-CD3z
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 251 - protein, 666 aa, Artificial Sequence
  hTIM4-TIM4-TIM4tm-TIM4ICD-CD3z-TLR2
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 252 - protein, 684 aa, Artificial Sequence
  hTIM4-TIM4-TIM4tm-TIM4ICD-TLR8-CD3z
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 253 - protein, 684 aa, Artificial Sequence
  hTIM4-TIM4-TIM4tm-TIM4ICD-CD3z-TLR8
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 254 - protein, 553 aa, Artificial Sequence
  hTIM4-TIM4-TIM4tm-TLR2ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 255 - protein, 571 aa, Artificial Sequence
  hTIM4-TIM4-TIM4tm-TLR8ICD
  SIGNAL (1)..(22)
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 256 - protein, 598 aa, Artificial Sequence
  HPV16 E7 TCRB chain-P2A-TCRa chain; amino acids 309-335 are P2A sequence
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 257 - protein, 496 aa, Artificial Sequence; identical to SEQ ID NO: 71
  pCTX247: Tim4 signal peptide - Tim4 ECD - CD28 TM - CD28 ICD - CD3z ICD

SEQ ID NO: 258 - protein, 496 aa, Artificial Sequence; identical to SEQ ID NO: 71
  pCTX797: Tim4 signal peptide - Tim4 ECD - CD28 TM - CD28 ICD - CD3z ICD

(19) United States
(12) Patent Application Publication
(10) Pub. No.: US 2025/0186491 A1
(43) Pub. Date: Jun. 12, 2025

(54) COMPOSITIONS AND METHODS FOR TREATING CANCER WITH CHIMERIC TIM RECEPTORS IN COMBINATION WITH INHIBITORS OF POLY (ADP-RIBOSE) POLYMERASE

(71) Applicant: CERO THERAPEUTICS HOLDINGS, INC., South San Francisco, CA (US)

(72) Inventors: Daniel Mark COREY, Menlo Park, CA (US); Nathan KIPNISS, San Francisco, CA (US); Geoff O'DONOGHUE, South San Francisco, CA (US)

(21) Appl. No.: 18/041,200

(22) PCT Filed: Aug. 13, 2021
(86) PCT No.: PCT/US2021/046041

§ 371 Date: Feb. 9, 2023

Related U.S. Application Data
(60) Provisional application No. 63/066,150, filed on Aug. 14, 2020.
(60) Provisional application No. 63/087,049, filed on Oct. 2, 2020.
(60) Provisional application No. 63/226,712, filed on Jul. 28, 2021.

(51) Int. Cl. A61K 35/17; A61K 31/4184; A61K 31/454; A61K 31/502; A61K 31/5025; A61K 31/55; A61K 40/11; A61K 40/41; A61K 40/42; C07K 14/705; C07K 14/725; C12N 5/0783

(52) U.S. Cl. CPC A61K 35/17; A61K 31/4184; A61K 31/454; A61K 31/502; A61K 31/5025; A61K 31/55; A61K 40/11; A61K 40/421; A61K 40/4285; C07K 14/7051; C07K 14/70521; C12N 5/0636; A61K 2239/59; C07K 2319/02; C07K 2319/03; C12N 2510/00

(57) ABSTRACT

The present disclosure relates to combination therapy compositions and methods comprising chimeric Tim receptors, host cells modified to include chimeric Tim receptor molecules, and PARP inhibitors.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 200265_415WO_ST25.txt. The text file is 425 KB, was created on Aug. 13, 2021, and is being submitted electronically via EFS-Web.

### BACKGROUND

Upon exposure to antigen, naïve antigen-specific CD8+ T cells undergo activation that promotes their clonal expansion, differentiation, and development into functional, effector T cells that can kill cells expressing the cognate antigen (e.g., tumor cells). Following antigen clearance, the majority of effector T cells undergo apoptosis, and a subset of the surviving effector T cells differentiate into memory T cells that can confer long-term protection against antigen re-exposure. However, prolonged antigen exposure may result in T cell exhaustion, enabling the persistence of tumor cells. T cell exhaustion refers to a dysfunctional state acquired by T cells experiencing persistent TCR stimulation characterized by upregulated expression of immune checkpoint molecules (e.g., PD-1, CTLA-4, Tim-3), impaired effector function, poor proliferation, and metabolic defects. Engineered T cells expressing chimeric antigen receptors (CARs) can also develop exhaustion.

### BRIEF DESCRIPTION OF THE SEVERAL VIEWS OF THE DRAWINGS

FIGS. 1A-1B show that the Kuramochi model of ovarian cancer is moderately sensitive to PARP inhibition. FIG. 1A shows the results of Kuramochi cells expressing an mCherry-NLS plasmid incubated with the PARP inhibitor Niraparib. FIG. 1B shows the results of Kuramochi cells expressing an mCherry-NLS plasmid incubated with the PARP inhibitors Niraparib, Olaparib, Talazoparib, Veliperib, and Rucaparib.

FIGS. 2A-2C show Kuranochi cells challenged with Niraparib bind Tim4, indicating increased levels of surface PS in response to PARP inhibition. FIG. 2A shows staining of Niraparib treated Kuramochi cells by Tim4-Fc chimera and anti-mouse IgG2a antibody conjugated to Alexa488. FIG. 2B shows staining for IgG control. FIG. 2C shows increase in Kuramochi cell number in response to 1.56-12.5 μM Niraparib.

FIG. 3 shows that Niraparib has negligible impact on CD4/CD8 T cell health, cell size, or expansion.

FIG. 4 shows the results of Kuramochi cells were incubated with chimeric Tim receptor Construct 13A, control T cells, and Niraparib.

FIG. 5 shows that combination of chimeric Tim4 receptor (CTX140) and niraparib treatment reduces Kuramochi cell numbers in vitro. Kuramochi cells were pre-incubated with 1.56 μM niraparib. CTX156 (tEGFR) is a transduction control.

FIG. 6 shows that combination of chimeric Tim4 receptor (CTX137) and niraparib treatment reduces Kuramochi cell numbers in vitro. Kuramochi cells were pre-incubated with 25 μM niraparib and given maintenance dose of 0.52 μM nariparib. CTX156 (tEGFR) is a transduction control.

FIG. 7 shows that combination of chimeric Tim4 receptor (CTX137) with nirparib treatment at different doses (6.25 μM, 12.5 μM, or 25 μM pre-treatment+0.52 μM maintenance dose) and different effector:target cell ratios reduces Kuramochi cell numbers in vitro. CTX156 (tEGFR) is a transduction control.

FIGS. 8A-8B show that pCTX133, a TLR-2 containing Chimeric Tim4 Receptor Enhances Potency of Niraparib in an Ovarian Cancer Model. FIG. 8A: Flow cytometry measurement of surface PtdSer. Kuramochi cells were treated with 1.56 or 25 μM Niraparib or with equivalent volume of DMSO (control). 48 hours later samples were trypsinized and stained using a Tim4-Fc followed by a fluorescently-labeled secondary anti body to the Tim4-Fc. FIG. 8B: Kuramochi cells pre-treated for ˜20 hours with 1.56 μM Niraparib were co-cultured with pCTX133 and Untransduced CD4 T cells from donor 32 at a 2:1 T cell:Kuramochi ratio and a final Niraparib concentration of 1.56 μM. Samples treated with Niraparib+pCTX133 exhibited substantially fewer tumor cells in culture ˜3 days later when compared to samples treated with Niraparib alone, or Niraparib+untransduced T cells. All data were collected via IncuCyte.

FIGS. 9A-9B: CER pCTX247 (SEQ ID NO:257) in vitro anti-tumor activity in two Ovarian cancer models. FIG. 9A: A2780-mCh-NLS cells pre-treated for ˜20 hours with 25 μM Niraparib were cocultured with CER 247 and MOCK 236 CD4/8 T cells from donor 21 at a 2:1 T cell:A2780 ratio and a final Niraparib concentration of 6.25 μM. Samples treated with Niraparib+CER 247 exhibited substantially fewer tumor cells in culture ˜5 days later when compared to samples treated with Niraparib alone, CER 247 alone, or Niraparib+MOCK 236. FIG. 9B: Kuramochi-mCh-NLS cells pre-treated for ˜20 hours with 25 μM Niraparib were cocultured with CER 247 and MOCK 236 CD4/8 T cells from donor 21 at a 2:1 T cell:A2780 ratio and a final Niraparib concentration of 1.56 μM. Samples treated with Niraparib+CER 247 exhibited substantially fewer tumor cells in culture ˜5 days later when compared to samples treated with Niraparib alone, CER 247 alone, or Niraparib+MOCK 236. All data were collected via IncuCyte.

FIG. 10: CER pCTX797 (SEQ ID NO:258) in vitro antitumor activity in the A2780 Ovarian cancer model. A2780-mCh-NLS cells pre-treated for ˜20 hours with 0.52 μM Niraparib were co-cultured with CER 797 and MOCK 236 CD4/8 T cells from donor 32 at a 2:1 T cell:A2780 ratio and a final Niraparib concentration of 0.52 LIM. Samples treated with Niraparib+CER 247 exhibited substantially fewer tumor cells in culture ˜5 days later when compared to samples treated with Niraparib alone, CER 247 alone, or Niraparib+MOCK 236. All data were collected via IncuCyte.

FIGS. 11A-11B: Phosphatidylserine is present on the cell surface in response to Niraparib in two Ovarian cancer models. FIG. 11A: A2780-mCh-NLS cells treated with 1.56 and 25 μM Niraparib exhibit a significantly higher fraction of PS+ cells and an increase in Tim4-Fc MFI compared to samples treated with equivalent volumes of DMSO. Niraparib also has an anti-tumor effect beginning at a concentration of at least 1.56 μM Niraparib. FIG. 11B: Kuramochi-mCh-NLS cells treated with 25 μM Niraparib exhibit a significantly higher fraction of PS+ cells and an increase in Tim4-Fc MFI compared to samples treated with equivalent volumes of DMSO. Niraparib also has an anti-tumor effect beginning at a concentration of at least 25 μM Niraparib. All samples were analyzed via Flow Cytometry.

FIG. 12: CERs exhibit synergy with additional PARP inhibitors (Rucaparib and Olaparib, in addition to Niraparib). A2780-mCh-NLS cells pre-treated for ˜20 hours with 0.52 μM Niraparib, Rucaparib, or Olaparib were co-cultured with CER 247 and MOCK 236 CD4/8 T cells from donor 32 at a 2:1 T cell:A2780 ratio and a final Niraparib concentration of 0.52 μM. Samples treated with Niraparib+CER 247, Rucaparib+CER247, or Olaparib+CER 247 exhibited substantially fewer tumor cells in culture ˜5 days later when compared to samples treated with Niraparib, Rucaparib, or Olaparib alone, CER 247 alone, or Niraparib+MOCK 236. All data were collected via IncuCyte.

### DETAILED DESCRIPTION

In one aspect, the present disclosure provides methods for the treatment of cancer using chimeric T-cell immunoglobulin mucin protein (Tim) receptors, also referred to as chimeric engulfment receptors (CERs), in combination with an inhibitor of Poly (ADP-ribose) polymerase (PARP). In another aspect, the present disclosure provides a pharmaceutical compositions or combination comprising a Chimeric Tim receptor and a PARP inhibitor. Chimeric Tim receptors useful in compositions and methods of the present disclosure confer engulfment, cytotoxicity, and/or antigen presentation activity to chimeric Tim receptor-modified host cells (e.g., T cells), with the cytotoxic activity being induced upon binding of the chimeric Tim receptor to its target antigen, phosphatidylserine. DNA damaging agents such as PARP inhibitors may synergize with chimeric Tim receptors by promoting cell damage and externalization of phosphatidylserine, which induces effector function of chimeric Tim receptors, such as engulfment, cytotoxicity, costimulatory activity, antigen presentation, or a combination thereof.

Chimeric Tim receptors described herein comprise a single chain chimeric protein, the single chain chimeric protein comprising: (a) an extracellular domain comprising a binding domain comprising: (i) a Tim1 IgV domain or a Tim4 IgV domain; and (ii) a Tim1 mucin domain or a Tim4 mucin domain; (b) an intracellular signaling domain, wherein the intracellular signaling domain comprises a primary intracellular signaling domain and optionally a secondary intracellular signaling domain; and (c) a transmembrane domain positioned between and connecting the extracellular domain and the intracellular signaling domain.

In certain embodiments, the extracellular domain of the chimeric Tim receptors described herein optionally includes an extracellular spacer domain positioned between and connecting the binding domain and transmembrane domain.

In certain embodiments, chimeric Tim receptors may also be capable of costimulating T cells via a different signaling pathway than the “classical” f cell costimulation pathways (e.g., CD28). For example, in addition to binding phosphatidylserine, Tim4 is also a ligand for Tim1, which is expressed on the surface of activated T cells. Tim1 is also capable of binding to phosphatidylserine. Tim4-induced Tim1 signaling has been found to costimulate T cell proliferation and survival (Hartt Meyers et al., 2005, Nat. Immunol. 6:455). Thus, in certain embodiments, cytotoxic chimeric Tim receptors may reduce or inhibit T cell exhaustion, or restore exhausted T cells by providing costimulatory signals via at least one signaling pathway. In certain embodiments, cytotoxic chimeric Tim receptors provide costimulatory signals via at least two distinct signaling pathways (e.g., via the selected costimulatory signaling domain in the cytotoxic chimeric Tim receptor and Tim1).

In certain embodiments, when expressed in a host cell, the chimeric Tim receptors of the present disclosure also confer engulfment activity to the host cell. For example, in certain such embodiments, binding of the chimeric Tim receptor expressed in a host cell to a phosphatidylserine target may induce both cytolytic and engulfment responses by the host cell. In particular embodiments of the modified host cells described herein, the host cell does not naturally exhibit an engulfment phenotype prior to modification with the chimeric Tim receptor.

In embodiments, administration of host cells modified with chimeric Tim receptors of the present disclosure can be used in methods for eliminating target cells bearing surface exposed phosphatidylserine, e.g., for the treatment of cancer. In normal, healthy cells phosphatidylserine is located in the inner leaflet of the plasma membrane. However, certain cellular events, such as damage, apoptosis, necrosis, and stress, activates a “scramblase” that quickly exposes phosphatidylserine on the cell surface, where it can bind to receptors such as Tim4 or Tim1. Endogenous tumor-specific effector T cells can induce exposure of phosphatidylserine on the outer membrane of targeted tumor cells during cytolysis. Furthermore, certain cancer therapies (e.g., chemotherapy, radiotherapy, CAR-T cells, etc.) can induce exposure of phosphatidylserine on targeted tumor cells or cells in the tumor microenvironment by inducing apoptosis, cellular stress, cellular damage, etc. Engineered host cells expressing the presently disclosed chimeric Tim receptors may clear damaged, stressed, apoptotic, or necrotic tumor cells bearing surface exposed phosphatidylserine by inducing apoptosis in the tumor cells bearing surface exposed phosphatidylserine. In certain embodiments, host cells expressing chimeric Tim receptors disclosed herein clear damaged, stressed, apoptotic, or necrotic tumor cells bearing surface exposed phosphatidylserine by inducing apoptosis and by engulfment. In another aspect, host cells modified with chimeric Tim receptors of the present disclosure can be used to enhance the effect of a PARP inhibitor that induces cellular stress, damage, necrosis, or apoptosis. For example, administration of a PARP inhibitor may increase levels of surface phosphatidylserine, thus resulting in a synergistic combination. Cells expressing a chimeric Tim receptor as presently described can bind to the phosphatidylserine moieties exposed on the outer leaflet of damaged or dying cells resulting from administration of the PARP inhibitor and induce cytolysis or both cytolysis and engulfment of the targeted cells.

The combination of engineered host cells comprising chimeric Tim receptors and a PARP inhibitor according to the present description may be administered to a subject alone, or in further combination with one or more additional therapeutic agents, including for example CAR-T cells, TCRs, antibodies, radiation therapy, chemotherapies, small molecules, oncolytic viruses, electropulse therapy, etc.

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g. “or”) should be understood to mean either one, both, or any combination thereof of the alternatives. As used herein, the terms “include,” “have” and “comprise” are used synonymously, which terms and variants thereof are intended to be construed as non-limiting.

Terms understood by those in the art of antibody technology are each given the meaning acquired in the art, unless expressly defined differently herein. The term “antibody” is used in the broadest sense and includes polyclonal and monoclonal antibodies. An “antibody” may refer to an intact antibody comprising at least two heavy (H) chains and two light (L) chains inter-connected by disulfide bonds, as well as an antigen-binding portion (or antigen-binding domain) of an intact antibody that has or retains the capacity to bind a target molecule. An antibody may be naturally occurring, recombinantly produced, genetically engineered, or modified forms of immunoglobulins, for example intrabodies, peptibodies, nanobodies, single domain antibodies, SMIPs, multispecific antibodies (e.g., bispecific antibodies, diabodies, triabodies, tetrabodies, tandem di-scFV, tandem tri-scFv, ADAPTIR). A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). “Antigen-binding portion” or “antigen-binding domain” of an intact antibody is meant to encompass an “antibody fragment,” which indicates a portion of an intact antibody and refers to the antigenic determining variable regions or complementary determining regions of an intact antibody. Examples of antibody fragments include, but are not limited to, Fab, Fab′, F(ab′), and Fv fragments, Fab′-SH, F(ab′)₂, diabodies, linear antibodies, scFv antibodies, VH, and multispecific antibodies formed from antibody fragments. A “Fab” (fragment antigen binding) is a portion of an antibody that binds to antigens and includes the variable region and CH1 of the heavy chain linked to the light chain via an inter-chain disulfide bond. An antibody may be of any class or subclass, including IgG and subclasses thereof (IgG₁, IgG₂, IgG₃, IgG₄), IgM, IgE, IgA, and IgD.

The term “variable region” or “variable domain” refers to the domain of an antibody heavy or light chain that is involved in binding of the antibody to antigen. The variable domains of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an anti body that binds the antigen to screen a library of complementary VL or VH domains, respectively. See, e.g., Portolano et al., J. Immunol. 150:880-887 (1993); Clarkson et al., Nature 352:624-628 (1991).

The terms “complementarity determining region” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (HCDR1, HCDR2, HCDR3) and three CDRs in each light chain variable region (LCDR1, LCDR2, LCDR3).

As used herein, the terms “binding domain”, “binding region”, and “binding moiety” refer to a molecule, such as a peptide, oligopeptide, polypeptide, or protein that possesses the ability to specifically and non-covalently bind, associate, unite, recognize, or combine with a target molecule (e.g., phosphatidylserine). A binding domain includes any naturally occurring, synthetic, semi-synthetic, or recombinantly produced binding partner for a biological molecule or other target of interest. In some embodiments, the binding domain is an antigen-binding domain, such as an antibody or functional binding domain or antigen-binding portion thereof. Exemplary binding domains include single chain antibody variable regions (e.g. domain antibodies, sFv, scFv, Fab), receptor ectodomains (e.g., Tim4), ligands (e.g., cytokines, chemokines), or synthetic polypeptides selected for the specific ability to bind to a biological molecule.

“T cell receptor” (TCR) refers to a molecule found on the surface of T cells (also referred to as T lymphocytes) that is generally responsible for recognizing antigens bound to major histocompatibility complex (MI-C) molecules. The TCR is generally composed of a disulfide-linked heterodimer of the highly variable α and β chains (also known as TCRα and TCRβ, respectively) in most T cells. In a small subset of T cells, the TCR is made up of a heterodimer of γ and δ chains (also known as TCRγ and TCRδ, respectively). Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Janeway et al. Immunobiology: The Immune System in Health and Disease, 3^{rd} Ed., Current Biology Publications, p. 4:33, 1997). TCRs of the present disclosure may be from various animal species, including human, mouse, rat, cat, dog, goat, horse, or other mammals. TCRs may be cell-bound (i.e., have a transmembrane region or domain) or in soluble form. TCRs include recombinantly produced, genetically engineered, fusion, or modified forms of TCRs, including for example, scTCRs, soluble TCRs, TCR fusion constructs (TRuC™; see, U.S. Patent Publication No. 2017/0166622).

The term “variable region” or “variable domain” of a TCR α-chain (Vα) and β-chain (Vβ), or Vγ and Vδ for γδ TCRs, are involved in binding of the TCR to antigen. The V_{α} and V_{β} of a native TCR generally have similar structures, with each variable domain comprising four conserved FRs and three CDRs. The V_{α} domain is encoded by two separate DNA segments, the variable gene segment (V gene) and the joining gene segment (J gene); the V_{β} domain is encoded by three separate DNA segments, the variable gene segment (V gene), the diversity gene segment (D gene), and the joining gene segment (J gene). A single V_{α} or V_{β} domain may be sufficient to confer antigen-binding specificity. “Major histocompatibility complex molecule” (MHC molecule) refers to a glycoprotein that delivers a peptide antigen to a cell surface. MHC class I molecules are heterodimers composed of a membrane spanning α chain (with three α domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, or other mammals.

“Chimeric antigen receptor” (CAR) refers to a chimeric protein comprising two or more distinct domains and can function as a receptor when expressed on the surface of a cell. CARs are generally composed of an extracellular domain comprising a binding domain that binds a target antigen, an optional extracellular spacer domain, a transmembrane domain, and an intracellular signaling domain (e.g., an immunoreceptor tyrosine-based activation motif (ITAM)-containing T cell activating motif, and optionally an intracellular costimulatory domain). In certain embodiments, an intracellular signaling domain of a CAR has an ITAM-containing T cell activating domain (e.g., CD3ζ) and an intracellular costimulatory domain (e.g., CD28). In certain embodiments, a CAR is synthesized as a single polypeptide chain or is encoded by a nucleic acid molecule as a single chain polypeptide.

A variety of assays are known for identifying binding domains of the present disclosure that specifically bind a particular target, as well as determining binding domain affinities, such as Western blot, ELISA, and BIACORE® analysis (see also, e.g., Scatchard et al., Ann. N.Y. Acad. Sci. 51.660, 1949; and U.S. Pat. Nos. 5,283,173, 5,468,614, or the equivalent). As used herein, “specifically binds” refers to an association or union of a binding domain, or a fusion protein thereof, to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample.

The terms “antigen” and “Ag” refer to a molecule that is capable of inducing an immune response. The immune response that is induced may involve antibody production, the activation of specific immunologically-competent cells, or both. Macromolecules, including proteins, glycoproteins, and glycolipids, can serve as an antigen. Antigens can be derived from recombinant or genomic DNA. As contemplated herein, an antigen need not be encoded (i) solely by a full length nucleotide sequence of a gene or (ii) by a “gene” at all. An antigen can be generated or synthesized, or an antigen can be derived from a biological sample. Such a biological sample can include, but is not limited, to a tissue sample, a tumor sample, a cell, or a biological fluid.

The term “epitope” or “antigenic epitope” includes any molecule, structure, amino acid sequence or protein determinant within an antigen that is specifically bound by a cognate immune binding molecule, such as an antibody or fragment thereof (e.g., scFv), T cell receptor (TCR), chimeric Tim receptor, or other binding molecule, domain or protein. Epitopic determinants generally contain chemically active surface groupings of molecules, such as amino acids or sugar side chains, and can have specific three dimensional structural characteristics, as well as specific charge characteristics. An epitope may be a linear epitope or a conformational epitope.

As used herein, the term “PARP” (Poly (ADP-ribose) polymerase) refers to a family of proteins, each of which have two ribose moieties and two phosphates per unit polymer. PARP1 and PARP2 are enzymes involved in a DNA repair pathway.

As used herein, the term “Tim4” (T-cell immunoglobulin and mucin domain containing protein 4), also known as “TimD4”, refers to a phosphatidylserine receptor that is typically expressed on antigen presenting cells, such as macrophages and dendritic cells. Tim4 mediates the phagocytosis of apoptotic, necrotic, damaged, injured, or stressed cells, which present phosphatidylserine (PtdSer) on the exofacial (outer) leaflet of the cell membrane. Tim4 is also capable of binding to Tim1 expressed on the surface of T cells and inducing proliferation and survival. In certain embodiments, Tim4 refers to human Tim4. An exemplary human Tim4 protein comprises an amino acid sequence of SEQ ID NO:1.

As used herein, the term “Tim4 binding domain” refers to the N-terminal immunoglobulin-fold domain of Tim4 that possesses a metal ion-dependent pocket that selectively binds PtdSer. An exemplary human Tim4 binding domain comprises an amino acid sequence of SEQ ID NO:2, and an exemplary mouse Tim4 binding domain comprises an amino acid sequence of SEQ ID NO:24.

A Tim4 binding domain includes a variable immunoglobulin (IgV) like domain (referred to herein as an “IgV domain”) and a Mucin like domain (“referred to herein as a “mucin domain”). An exemplary human Tim4 IgV domain comprises an amino acid sequence of SEQ ID NO:34, and an exemplary human Tim4 mucin domain comprises an amino acid sequence of SEQ ID NO:35. In certain embodiments, the Tim4 binding domain does not include a signal peptide. An exemplary human Tim4 signal peptide has the amino acid sequences of SEQ ID NO: 11. An exemplary mouse Tim4 signal peptide has the amino acid sequences of SEQ ID NO:25.

As used herein, the term “Tim1” (T-cell immunoglobulin and mucin domain containing protein 1), refers to a phosphatidylserine receptor that is expressed on the surface of T cells. Tim1, as noted above is also capable of binding to Tim4 expressed on the surface of antigen presenting cells. In certain embodiments. Tim1 refers to human Tim1. An exemplary human Tim1 protein comprises an amino acid sequence of SEQ LD NO:36.

As used herein, the term “Tim1 binding domain” refers to the N-terminal immunoglobulin-fold domain of Tim1 that selectively binds PtdSer. An exemplary human Tim1 binding domain comprises an amino acid sequence of SEQ ID NO:37.

A Tim1 binding domain includes an IgV domain and a mucin domain. An exemplary human Tim1 IgV domain comprises an amino acid sequence of SEQ ID NO:38, and an exemplary human Tim₁ mucin domain comprises an amino acid sequence of SEQ ID NO:39. In certain embodiments, the Tim1 binding domain does not include a signal peptide. An exemplary human Tim1 signal peptide has the amino acid sequences of SEQ ID NO:40.

As used herein, an “effector domain” is an intracellular portion of a fusion protein or receptor that can directly or indirectly promote a biological or physiological response in a cell expressing the effector domain when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response.

As used herein, a “costimulatory signaling domain” refers to an intracellular signaling domain, or functional portion thereof, of a costimulatory molecule, which, when activated in conjunction with a primary or classic (e.g., ITAM-driven) activation signal (provided by, for example, a CD3ζ intracellular signaling domain), promotes or enhances a T cell response, such as T cell activation, cytokine production, proliferation, differentiation, survival, effector function, or combinations thereof. Costimulatory signaling domains include, for example, CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (H-EM), CD273 (PD-L2), CD274 (PD-L1), CD278 (ICOS), DAP10, LAT, LFA-1, LIGHT, NKG2C, SLP76, TRIM, or any combination thereof.

As used herein, an “immunoreceptor tyrosine-based activation motif (ITAM) activating domain” refers to an intracellular signaling domain or functional portion thereof which is naturally or endogenously present on an immune cell receptor or a cell surface marker and contains at least one immunoreceptor tyrosine-based activation motif (ITAM). ITAM refers to a conserved motif of YXXL/I-X₆₋₈-YXXL/I. In certain embodiments an ITAM signaling domain contains one, two, three, four, or more ITAMs. An ITAM signaling domain may initiate T cell activation signaling following antigen binding or ligand engagement. ITAM-signaling domains include, for example, intracellular signaling domains of CD3γ, CD3δ, CD3ε, CD3ζ, CD79a, and CD66d.

“Junction amino acids” or “junction amino acid residues” refer to one or more (e.g., about 2-20) amino acid residues between two adjacent motifs, regions or domains of a polypeptide. Junction amino acids may result from the construct design of a chimeric protein (e.g., amino acid residues resulting from the use of a restriction enzyme site during the construction of a nucleic acid molecule encoding a chimeric protein).

“Nucleic acid molecule” and “polynucleotide” can be in the form of RNA or DNA, which includes cDNA, genomic DNA, and synthetic DNA. A nucleic acid molecule may be composed of naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

“Encoding” refers to the inherent property of specific polynucleotide sequences, such as DNA, cDNA, and mRNA sequences, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a polynucleotide encodes a protein if transcription and translation of mRNA corresponding to that polynucleotide produces the protein in a cell or other biological system. Both a coding strand and a non-coding strand can be referred to as encoding a protein or other product of the polynucleotide. Unless otherwise specified, a “nucleotide sequence encoding an amino acid sequence” includes all nucleotide sequences that are degenerate versions of each other and that encode the same amino acid sequence.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “mature polypeptide” or “mature protein” refers to a protein or polypeptide that is secreted or localized in the cell membrane or inside certain cell organelles (e.g., the endoplasmic reticulum, golgi, or endosome) and does not include an N-terminal signal peptide.

A “signal peptide”, also referred to as “signal sequence”, “leader sequence”, “leader peptide”, “localization signal” or “localization sequence”, is a short peptide (usually 15-30 amino acids in length) present at the N-terminus of newly synthesized proteins that are destined for the secretory pathway. A signal peptide typically comprises a short stretch of hydrophilic, positively charged amino acids at the N-terminus, a central hydrophobic domain of 5-15 residues, and a C-terminal region with a cleavage site for a signal peptidase. In eukaryotes, a signal peptide prompts translocation of the newly synthesized protein to the endoplasmic reticulum where it is cleaved by the signal peptidase, creating a mature protein that then proceeds to its appropriate destination.

The term “chimeric” refers to any nucleic acid molecule or protein that is not endogenous and comprises sequences joined or linked together that are not normally found joined or linked together in nature. For example, a chimeric nucleic acid molecule may comprise regulatory sequences and coding sequences that are derived from different sources, or regulatory sequences and coding sequences that are derived from the same source but arranged in a manner different than that found in nature.

As used herein, the term “endogenous” or “native” refers to a gene, protein, compound, molecule, or activity that is normally present in a host or host cell, including naturally occurring variants of the gene, protein, compound, molecule, or activity.

As used herein, “homologous” or “homolog” refers to a molecule or activity from a host cell that is related by ancestry to a second gene or activity, e.g., from the same host cell, from a different host cell, from a different organism, from a different strain, from a different species. For example, a heterologous molecule or heterologous gene encoding the molecule may be homologous to a native host cell molecule or gene that encodes the molecule, respectively, and may optionally have an altered structure, sequence, expression level or any combination thereof.

As used herein, “heterologous” nucleic acid molecule, construct or sequence refers to a nucleic acid molecule or portion of a nucleic acid molecule that is not native to a host cell, but can be homologous to a nucleic acid molecule or portion of a nucleic acid molecule from the host cell. The source of the heterologous nucleic acid molecule, construct or sequence can be from a different genus or species. In some embodiments, the heterologous nucleic acid molecules are not naturally occurring. In certain embodiments, a heterologous nucleic acid molecule is added (i.e., not endogenous or native) into a host cell or host genome by, for example, conjugation, transformation, transfection, transduction, electroporation, or the like, wherein the added molecule can integrate into the host cell genome or exist as extra-chromosomal genetic material (e.g., as a plasmid or other form of self-replicating vector), and can be present in multiple copies. In addition, “heterologous” refers to a non-native enzyme, protein or other activity encoded by a non-endogenous nucleic acid molecule introduced into the host cell, even if the host cell encodes a homologous protein or activity.

As used herein, the term “engineered,” “recombinant,” “modified” or “non-natural” refers to an organism, microorganism, cell, nucleic acid molecule, or vector that has been modified by introduction of a heterologous nucleic acid molecule, or refers to a cell or microorganism that has been genetically engineered by human intervention that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins, chimeric receptors, or enzymes, or other nucleic acid molecule additions, deletions, substitutions, or other functional disruption of or addition to a cell's genetic material. Exemplary modifications include those in coding regions or functional fragments thereof heterologous or homologous polypeptides from a reference or parent molecule. Additional exemplary modifications include, for example, modifications in non-coding regulatory regions in which the modifications alter expression of a gene or operon.

As used herein, the term “transgene” refers to a gene or polynucleotide encoding a protein of interest (e.g., chimeric Tim receptor) whose expression is desired in a host cell and that has been transferred by genetic engineering techniques into a cell. A transgene may encode proteins of therapeutic interest as well as proteins that are reporters, tags, markers, suicide proteins, etc. A transgene may be from a natural source, modification of a natural gene, or a recombinant or synthetic molecule. In certain embodiments, a transgene is a component of a vector.

The term “overexpressed” or “overexpression” of an antigen refers to an abnormally high level of antigen expression in a cell. Overexpressed antigen or overexpression of antigen is often associated with a disease state, such as in hematological malignancies and cells forming a solid tumor within a specific tissue or organ of a subject. Solid tumors or hematological malignancies characterized by overexpression of a tumor antigen can be determined by standard assays known in the art.

The “percent identity” between two or more nucleic acid or amino acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al, J. Mol. Biol 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8).

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may, for example, be one that expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that a promoter is in the correct location and orientation in relation to a polynucleotide to control the initiation of transcription by RNA polymerase and expression of the polynucleotide.

A “vector” is a nucleic acid molecule that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment.

In certain embodiments, the vector is a viral vector. Examples of viral vectors include, but are not limited to, adenovirus vectors, adeno-associated virus vectors, retrovirus vectors, gamma retrovirus vectors, and lentivirus vectors. “Retroviruses” are viruses having an RNA genome. “Gamma retrovirus” refers to a genus of the retroviridae family. Examples of gamma retroviruses include mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV).

In other embodiments, the vector is a non-viral vector. Examples of non-viral vectors include lipid-based DNA vectors, modified mRNA (modRNA), self-amplifying mRNA, closed-ended linear duplex (CELiD) DNA, and transposon-mediated gene transfer (PiggyBac, Sleeping Beauty). Where a non-viral delivery system is used, the delivery vehicle can be a liposome. Lipid formulations can be used to introduce nucleic acids into a host cell in vitro, ex vivo, or in vivo. The nucleic acid may be encapsulated in the interior of a liposome, interspersed within the lipid bilayer of a liposome, attached to a liposome via a linking molecule that is associated with both the liposome and the nucleic acid, contained or complexed with a micelle, or otherwise associated with a lipid.

As used herein, the term “engulfment” refers to a receptor-mediated process wherein endogenous or exogenous cells or particles greater than 100 nm in diameter are internalized by a phagocyte or host cell of the present disclosure. Engulfment is typically composed of multiple steps: (1) tethering of the target cell or particle via binding of an engulfment receptor to a pro-engulfment marker or antigenic marker directly or indirectly (via a bridging molecule) on a target cell or particle; and (2) internalization or engulfment of the whole target cell or particle, or a portion thereof. In certain embodiments, internalization may occur via cytoskeletal rearrangement of a phagocyte or host cell to form a phagosome, a membrane-bound compartment containing the internalized target. Engulfment may further include maturation of the phagosome, wherein the phagosome becomes increasingly acidic and fuses with lysosomes (to form a phagolysosome), whereupon the engulfed target is degraded (e.g., “phagocytosis”). Alternatively, phagosome-lysosome fusion may not be observed in engulfment. In yet another embodiment, a phagosome may regurgitate or discharge its contents to the extracellular environment before complete degradation. In some embodiments, engulfment refers to phagocytosis. In some embodiments, engulfment includes tethering of the target cell or particle by the phagocyte of host cell of the present disclosure, but not internalization. In some embodiments, engulfment includes tethering of the target cell or particle by the phagocyte of host cell of the present disclosure and internalization of part of the target cell or particle.

As used herein, the term “phagocytosis” refers to an engulfment process of cells or large particles (≥0.5 μm) wherein tethering of a target cell or particle, engulfment of the target cell or particle, and degradation of the internalized target cell or particle occurs. In certain embodiments, phagocytosis comprises formation of a phagosome that encompasses the internalized target cell or particle and phagosome fusion with a lysosome to form a phagolysosome, wherein the contents therein are degraded. In certain embodiments, during phagocytosis, following binding of a chimeric Tim receptor expressed on a host cell of the present disclosure to a phosphatidylserine expressed by a target cell or particle, a phagocytic synapse is formed; an actin-rich phagocytic cup is generated at the phagocytic synapse; phagocytic arms are extended around the target cell or particle through cytoskeletal rearrangements; and ultimately, the target cell or particle is pulled into the phagocyte or host cell through force generated by motor proteins. As used herein, “phagocytosis” includes the process of “efferocytosis”, which specifically refers to the phagocytosis of apoptotic or necrotic cells in a non-inflammatory manner.

The term “immune system cell” or “immune cell” means any cell of the immune system that originates from a hematopoietic stem cell in the bone marrow. Hematopoietic stem cells give rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a γδ T cell, a regulatory T cell, a natural killer cell, and a dendritic cell. Macrophages and dendritic cells may also be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

The term “T cells” refers to cells of T cell lineage. “Cells of T cell lineage” refer to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage, CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells. The term “T cells” encompasses naïve T cells (CD45 RA+, CCR7+, CD62L+, CD27+, CD45RO−), central memory T cells (CD45RO⁺, CD62L⁺, CD8⁺), effector memory T cells (CD45RA+, CD45RO−, CCR7−, CD62L−, CD27−), mucosal-associated invariant T (MAIT) cells, Tregs, natural killer T cells, and tissue resident T cells.

The term “B cells” refers to cells of the B cell lineage. “Cells of B cell lineage” refer to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B cells (e.g., CD19⁺, CD72+, CD24+, CD20⁺), or a physiological, morphological, functional, or immunological feature specific for a B cell. For example, cells of the B cell lineage may be progenitor or precursor cells committed to the B cell lineage (e.g., pre-pro-B cells, pro-B cells, and pre-B cells); immature and inactivated B cells or mature and functional or activated B cells. Thus, “B cells” encompass naïve B cells, plasma cells, regulatory B cells, marginal zone B cells, follicular B cells, lymphoplasmacytoid cells, plasmablast cells, and memory B cells (e.g., CD27⁺, IgD⁻).

The term “cytotoxic activity,” also referred to as “cytolytic activity,” with respect to a cell (e.g., a T cell or NK cell) expressing an immune receptor (e.g., a TCR) or a chimeric Tim receptor according to the present disclosure on its surface, means that upon antigen-specific signaling (e.g., via the TCR, chimeric Tim receptor), the cell induces a target cell to undergo apoptosis. In some embodiments, a cytotoxic cell may induce apoptosis in a target cell via the release of cytotoxins, such as perforin, granzyme, and granulysin, from granules. Perforins insert into the target cell membrane and form pores that allow water and salts to rapidly enter the target cell. Granzymes are serine proteases that induce apoptosis in the target cell. Granulysin is also capable of forming pores in the target cell membrane and is a proinflammatory molecule. In some embodiments, a cytotoxic cell may induce apoptosis in a target cell via interaction of Fas ligand, which is upregulated on T cell following antigen-specific signaling, with Fas molecules expressed on the target cell. Fas is an apoptosis-signaling receptor molecule on the surface of a number of different cells.

The term “exhaustion” with respect to immune cells refers to a state of immune cell dysfunction defined by poor effector function (e.g., reduced cytokine production, reduced cytotoxic activity), reduced proliferative capacity, increased expression of immune checkpoint molecules, and a transcriptional state distinct from that of functional effector or memory cells. In certain embodiments, an exhausted immune cell becomes unresponsive to the presence of its target antigen. Immune cell exhaustion may result from chronic exposure to a target antigen (e.g., as may result from chronic infection) or when it enters an immunosuppressive environment (e.g., a tumor microenvironment). In certain embodiments, immune cell exhaustion refers to T cell exhaustion, NK cell exhaustion, or both. In certain embodiments, exhausted T cells exhibit; (a) increased expression of PD-1, TIGIT, LAG3, TIM3, or any combination thereof; (b) decreased production of IFN-γ, IL-2, TNF-α, or any combination thereof; or both (a) and (b). In certain embodiments, exhausted NK cells exhibit; (a) increased expression of PD-1, NKG2A, TIM3, or any combination thereof; (b) decreased production of IFN-γ, TNF-α, or both; or both (a) and (b).

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

The term “cancer” as used herein is defined as disease characterized by the rapid and uncontrolled growth of aberrant cells. The aberrant cells may form solid tumors or constitute a hematological malignancy. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “subject,” “patient” and “individual” are used interchangeably herein and are intended to include living organisms in which an immune response can be elicited (e.g., mammals). Examples of subjects include humans, primates, cows, horses, sheep, dogs, cats, mice, rats, rabbits, guinea pigs, pigs, and transgenic species thereof.

“Adoptive cellular immunotherapy” or “adoptive immunotherapy” refers to the administration of naturally occurring or genetically engineered disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

“Autologous” refers to any material (e.g., a graft of organ, tissue, cells) derived from the same subject to which it is later to be re-introduced.

“Allogeneic” refers to a graft derived from a different subject of the same species.

A “therapeutically effective amount” or “effective amount” ofa chimeric protein or cell expressing a chimeric protein of this disclosure (e.g., a chimeric Tim receptor or a cell expressing a chimeric Tim receptor) refers to that amount of protein or cells sufficient to result in amelioration of one or more symptoms of the disease, disorder, or undesired condition being treated. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously.

“Treat” or “treatment” or “ameliorate” refers to medical management of a disease, disorder, or undesired condition of a subject. In general, an appropriate dose or treatment regimen comprising a host cell expressing a chimeric protein of this disclosure is administered in an amount sufficient to elicit a therapeutic or prophylactic benefit. Therapeutic or prophylactic/preventive benefit includes improved clinical outcome; lessening or alleviation of symptoms associated with a disease, disorder, or undesired condition; decreased occurrence of symptoms; improved quality of life; longer disease-free status; diminishment of extent of disease, disorder, or undesired condition; stabilization of disease state; delay of disease progression; remission; survival; prolonged survival; or any combination thereof.

The term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with a cancerous condition. An “anti-tumor effect” can also be manifested by prevention of a hematological malignancy or tumor formation.

“Autoimmune disease” refers to a disorder that results from an autoimmune response. An autoimmune disease is the result of an inappropriately excessive response to a self-antigen. An autoimmune response may involve self-reactive B-cells that produce autoantibodies, self-reactive T-cells, or both. An “autoantibody” as used herein is an antibody produced by a subject that binds to a self-antigen also produced by the subject.

Additional definitions are provided throughout the present disclosure.

As noted above, methods of the disclosure are useful for treating cancer in a subject. Therapeutic agents of the disclosure (i.e., a chimeric Tim receptor and a PARP inhibitor), when administered together or sequentially, treat a cancer in a subject (e.g., (i) arrest the cancer's development; (ii) cause regression of the cancer; or (iii) relieve the symptoms resulting from the cancer).

In embodiments, the methods of treatment disclosed herein comprise administering an effective amount of a chimeric Tim receptor and a PARP inhibitor to the subject, thereby treating their cancer.

### Chimeric Tim Receptors

The present disclosure provides a chimeric Tim receptor comprising a single chain chimeric protein, the single chain chimeric protein comprising: an extracellular domain comprising a Tim binding domain; an intracellular signaling domain comprising a first costimulatory signaling domain; and a transmembrane domain positioned between and connecting the extracellular domain and intracellular signaling domain. In certain embodiments, the extracellular domain of the chimeric Tim receptors described herein optionally includes an extracellular spacer domain positioned between and connecting the binding domain and transmembrane domain.

When expressed in a host cell, chimeric Tim receptors of the present disclosure can confer a phosphatidylserine-specific, cytotoxic phenotype to the modified host cell (e.g., the host cell becomes cytotoxic to a stressed, damaged, injured, apoptotic, or necrotic cell expressing phosphatidylserine on its surface). In certain embodiments, the chimeric Tim receptors induce apoptosis in targeted cells via release of granzymes, perforin, granulysin, or any combination thereof. In further embodiments, cells expressing a chimeric Tim receptor according to the present description exhibit an engulfment phenotype specific to phosphatidylserine presenting cells.

The intracellular signaling domain can include one or more effector domains that are capable of transmitting functional signals to a cell in response to binding of the extracellular domain of the chimeric Tim receptor and phosphatidylserine. Signaling by the intracellular signaling domain(s) is triggered by binding of the extracellular domain to phosphatidylserine. The signals transduced by the intracellular signaling domain promote effector function of the chimeric Tim receptor containing cell. Examples of effector function include cytotoxic activity, secretion of cytokines, proliferation, anti-apoptotic signaling, persistence, expansion, engulfment of a target cell or particle expressing phosphatidylserine on its surface, antigen presentation, or any combination thereof.

In certain embodiments, the intracellular signaling domain comprises a first intracellular signaling domain. In further embodiments, the intracellular signaling domain comprises a first intracellular signaling domain and a second intracellular signaling domain. In some embodiments, the intracellular signaling domain comprises a first intracellular signaling domain, a second intracellular signaling domain, and a third intracellular signaling domain. Chimeric Tim receptors according to the present disclosure can be used in a variety of therapeutic methods where clearance of apoptotic, necrotic, damaged, or stressed cells is beneficial, while providing costimulation that enhances cellular immune response, reduces immune cell exhaustion, or both.

Component parts of the fusion proteins of the present disclosure are further described in detail herein.

### Extracellular Domain

A Tim4 binding domain suitable for use in a chimeric Tim4 receptor of the present disclosure may be any polypeptide or peptide derived from a Tim4 molecule that specifically binds phosphatidylserine. In certain embodiments, the Tim4 binding domain is derived from human Tim4. An exemplary human Tim4 molecule is provided in Uniprot. Ref. Q96H15 (SEQ ID NO:1). An exemplary human Tim4 binding domain comprises or consists of an amino acid sequence of SEQ ID NO:2 or amino acids 25-314 of SEQ ID NO:2. An exemplary mouse Tim4 binding domain comprises or consists of an amino acid sequence of SEQ ID NO:24 or amino acids 23-279 of SEQ ID NO:24. In certain embodiments, the Tim4 binding domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:2 or amino acids 25-314 of SEQ ID NO:2, or SEQ ID NO:24 or amino acids 23-2⁷⁹ of SEQ ID NO:24. In certain embodiments, the Tim4 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:2 or amino acids 25-314 of SEQ ID NO:2, or SEQ ID NO:24 or amino acids 23-279 of SEQ ID NO:24.

In certain embodiments, the extracellular domain optionally comprises an extracellular, non-signaling spacer or linker domain. Where included, such a spacer or linker domain may position the binding domain away from the host cell surface to further enable proper cell/cell contact, binding, and activation. When included in a chimeric receptor as described herein, an extracellular spacer domain is generally located between the extracellular binding domain and the transmembrane domain of the chimeric Tim4 receptor. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/01687, which hinge region is incorporated herein by reference in its entirety. In a particular embodiment, an extracellular spacer domain comprises a modified IgG₄ hinge region having an amino acid sequence of ESKYGPPCPPCP (SEQ ID NO:3) Other examples of hinge regions that may be used in the chimeric Tim4 receptors described herein include the hinge region from the extracellular regions of type 1 membrane proteins, such as CD8a, CD4, CD28 and CD7, which may be wild-type or variants thereof. In some embodiments, an extracellular spacer domain comprises a CD28 hinge region having an amino acid sequence of SEQ ID NO:32. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

In certain embodiments, an extracellular domain comprises polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, an extracellular domain is murine, human, or chimeric.

The intracellular signaling domain of a chimeric Tim4 receptor as described herein is an intracellular effector domain and is capable of transmitting functional signals to a cell in response to binding of the extracellular domain of the chimeric Tim4 receptor and phosphatidylserine. The signals transduced by the intracellular signaling domain promote effector function of the chimeric Tim4 receptor containing cell. Examples of effector function include cytotoxic activity, secretion of cytokines, proliferation, anti-apoptotic signaling, persistence, expansion, engulfment of a target cell or particle expressing phosphatidylserine on its surface, or any combination thereof.

In certain embodiments, an intracellular signaling domain comprises a costimulatory signaling domain. The costimulatory signaling domain may be any portion of a costimulatory signaling molecule that retains sufficient signaling activity. In some embodiments, a full length or full length intracellular component of a costimulatory signaling molecule is used. In some embodiments, a truncated portion of a costimulatory signaling molecule or intracellular component of a costimulatory signaling molecule is used, provided that the truncated portion retains sufficient signal transduction activity. In further embodiments, a costimulatory signaling domain is a variant of a whole or truncated portion of a costimulatory signaling molecule, provided that the variant retains sufficient signal transduction activity (i.e., is a functional variant).

In certain embodiments, the costimulatory signaling domain comprises a CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), PD-1, CD273 (PD-L2), CD274 (PD-L1), 137-13 (CD276), RIOS (CD278), DAP10, LAT, LFA-1 (CD11a/CD18), LIGHT, NKG2C, SLP76, TRIM, or ZAP70 signaling domain. In particular embodiments, the costimulatory signaling domain comprises an OX40, CD2, CD27, CD28, ICAM-1, LFA-1 (CD11a/CD18), ICOS (CD278), or 4-1BB (CD137) signaling domain. An exemplary CD28 costimulatory signaling domain comprises or consists of an amino acid sequence of SEQ ID NO:118 or 119. An exemplary OX40 costimulatory signaling domain comprises or consists of an amino acid sequence of SEQ ID NO:120. An exemplary CD2 costimulatory signaling domain comprises or consists of an amino acid sequence of SEQ ID NO:121. An exemplary 4-1BB costimulatory signaling domain comprises or consists of an amino acid sequence of SEQ ID NO: 122. An exemplary CD27 costimulatory signaling domain comprises or consists of an amino acid sequence of SEQ ID NO: 123. An exemplary ICAM-1 costimulatory signaling domain comprises or consists of an amino acid sequence of SEQ ID NO:124. An exemplary LFA-1 costimulatory signaling domain comprises or consists of an amino acid sequence of SEQ ID NO:125. An exemplary ICOS costimulatory signaling domain comprises or consists of an amino acid sequence of SEQ ID NO:126. An exemplary CD30 costimulatory signaling domain comprises or consists of an amino acid sequence of SEQ ID NO: 127. An exemplary CD40 costimulatory signaling domain comprises or consists of an amino acid sequence of SEQ ID NO: 128. An exemplary PD-1 costimulatory signaling domain comprises or consists of an amino acid sequence of SEQ ID NO:129. An exemplary CD7 costimulatory signaling domain comprises or consists of an amino acid sequence of SEQ ID NO:130. An exemplary LIGHT costimulatory signaling domain comprises or consists of an amino acid sequence of SEQ ID NO:131. An exemplary NKG2C costimulatory signaling domain comprises or consists of an amino acid sequence of SEQ ID NO: 132. An exemplary B7-H3 costimulatory signaling domain comprises or consists of an amino acid sequence of SEQ ID NO: 133. In certain embodiments, the costimulatory signaling domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:118-133. In certain embodiments, the costimulatory signaling domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of any one of SEQ ID NOS: 118-133. In certain embodiments, the intracellular signaling comprises a second costimulatory signaling domain. In preferred embodiments, the first costimulatory signaling domain and second costimulatory signaling domain are different.

In certain embodiments, the intracellular signaling domain further comprises an ITAM-containing activating domain. The ITAM-containing activating domain may recapitulate TCR signaling independently of endogenous TCR complexes. In certain embodiments, signaling via the ITAM-containing activating domain leads to mediation of a T cell response, including, but not limited to, proliferation, activation, differentiation, and the like. The ITAM-containing activating domain may be any portion of an ITAM-containing activating domain molecule that retains sufficient signaling activity. In some embodiments, a full length or full length intracellular component of an ITAM-containing activating domain molecule is used. In some embodiments, a truncated portion of an ITAM-containing activating domain molecule or intracellular component of an ITAM-containing activating domain molecule is used, provided that the truncated portion retains sufficient signal transduction activity. In further embodiments, an ITAM-containing activating domain is a variant of a whole or truncated portion of an ITAM-containing activating domain molecule, provided that the variant retains sufficient signal transduction activity (i.e., is a functional variant).

Examples of ITAM-containing activating domains that may be used in the chimeric Tim4 receptors of the present disclosure include those derived from CD3ζ, CD3γ, CD3δ, CD3ε, CD5, CD22, CD79a, CD278 (ICOS), and CD66d. In specific embodiments, the ITAM-containing activating domain is a CD3ζ signaling domain. An exemplary CD3ζ signaling domain comprises or consists of an amino acid sequence of SEQ ID NO:134 or 135. An exemplary CD3γ signaling domain comprises or consists of an amino acid sequence of SEQ ID NO: 136. An exemplary CD3δ signaling domain comprises or consists of an amino acid sequence of SEQ ID NO: 137. An exemplary CD3, signaling domain comprises or consists of an amino acid sequence of SEQ ID NO:138. An exemplary CD5 signaling domain comprises or consists of an amino acid sequence of SEQ ID NO:139. An exemplary CD22 signaling domain comprises or consists of an amino acid sequence of SEQ ID NO: 140. An exemplary CD79a signaling domain comprises or consists of an amino acid sequence of SEQ ID NO:141. An exemplary CD66d signaling domain comprises or consists of an amino acid sequence of SEQ LD NO:143. In certain embodiments, the ITAM-containing activating domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:134-141, and 143. In certain embodiments, the ITAM containing activating domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) of an amino acid sequence to any one of SEQ ID NOS:134-141, and 143.

In another embodiment, an intracellular signaling domain comprises a CD28 costimulatory signaling domain and a CD3ζ signaling domain. In another embodiment, an intracellular signaling domain comprises a 4-1BB costimulatory signaling domain and a CD3ζ signaling domain. In yet another embodiment, an intracellular signaling domain comprises a CD27 costimulatory signaling domain and a CD3ζ signaling domain. In another embodiment, an intracellular signaling domain comprises a ICOS costimulatory signaling domain and a CD3ζ signaling domain. In another embodiment, an intracellular signaling domain comprises a LFA-1 costimulatory signaling domain and a CD3ζ signaling domain. In another embodiment, an intracellular signaling domain comprises an OX40 costimulatory signaling domain and a CD3ζ signaling domain. In yet another embodiment, an intracellular signaling domain comprises a CD2 costimulatory signaling domain and a CD3ζ signaling domain. In still another embodiment, an intracellular signaling domain comprises an ICAM-1 costimulatory signaling domain and a CD3ζ signaling domain.

Intracellular signaling domains may be derived from a mammalian species, including humans, primates, cows, horses, goats, sheep, dogs, cats, mice, rats, rabbits, guinea pigs, pigs, and transgenic species thereof.

The transmembrane domain of a chimeric Tim4 receptor connects and is positioned between the extracellular domain and the intracellular signaling domain. The transmembrane domain is a hydrophobic alpha helix that transverses the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). In one embodiment, the transmembrane domain is selected from the same molecule as the molecule from which the extracellular domain is derived. In another embodiment, the transmembrane domain is selected from the same molecule as the molecule from which the intracellular signaling domain is derived. For example, a chimeric Tim4 receptor may comprise a Tim4 binding domain and a Tim4 transmembrane domain. In another example, a chimeric Tim4 receptor may comprise a CD28 transmembrane domain and a CD28 costimulatory signaling domain. In certain embodiments, the transmembrane domain and the extracellular domain are derived from different molecules; the transmembrane domain and the intracellular signaling domain are derived from different molecules; or the transmembrane domain, extracellular domain, and intracellular signaling domain are all derived from different molecules. Examples of transmembrane domains that may be used in chimeric Tim4 receptors of the present disclosure include transmembrane domains from Tim4, Tim1, CD3ζ, CD3γ, CD3δ, CD3ε, CD28, CD45, CD4, CD5, CD8, CD9, CD16, CD22, CD33, CD37, CD64, CD80, CD86, CD134, CD137, CD154, CD27, CD28, 4-1BB, OX40, CD30, CD40, PD-1, ICOS, LFA-1, CD2, CD7, LIGHT, NKG2C, and B7-H3. An exemplary Tim4 transmembrane domain comprises or consists of an amino acid sequence of SEQ ID NO:144 or 23. An exemplary Tim1 transmembrane domain comprises or consists of an amino acid sequence of SEQ ID NO:8. An exemplary CD28 transmembrane domain comprises or consists of an amino acid sequence of SEQ ID NO:145. An exemplary 4-1BB transmembrane domain comprises or consists of an amino acid sequence of SEQ ID NO:146. An exemplary OX40 transmembrane domain comprises or consists of an amino acid sequence of SEQ ID NO:147. An exemplary CD27 transmembrane domain comprises or consists of an amino acid sequence of SEQ ID NO: 148. An exemplary ICOS transmembrane domain comprises or consists of an amino acid sequence of SEQ ID NO: 149. An exemplary CD2 transmembrane domain comprises or consists of an amino acid sequence of SEQ ID NO:150. An exemplary LFA-1 transmembrane domain comprises or consists of an amino acid sequence of SEQ ID NO: 151. An exemplary CD30 transmembrane domain comprises or consists of an amino acid sequence of SEQ ID NO: 152. An exemplary CD40 transmembrane domain comprises or consists of an amino acid sequence of SEQ ID NO:153. An exemplary PD-1 transmembrane domain comprises or consists of an amino acid sequence of SEQ ID NO: 154. An exemplary CD7 transmembrane domain comprises or consists of an amino acid sequence of SEQ ID NO:155. An exemplary LIGHT transmembrane domain comprises or consists of an amino acid sequence of SEQ ID NO: 156. An exemplary NKG2C transmembrane domain comprises or consists of an amino acid sequence of SEQ ID NO:157. An exemplary B37-H3 transmembrane domain comprises or consists of an amino acid sequence of SEQ ID NO: 158. In certain embodiments, the transmembrane domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:8, 23 and 144-158. In certain embodiments, the transmembrane domain comprises an amino acid sequence having at least about 1, 2, 3, 4, 5, 6, 7, 8, 9, or 10 amino acid modifications (e.g., deletion, additions, substitutions) to an amino acid sequence of any one of SEQ ID NOS:8, 23 and 144-158.

Transmembrane domains may derived from any mammalian species, including humans, primates, cows, horses, goats, sheep, dogs, cats, mice, rats, rabbits, guinea pigs, pigs, and transgenic species thereof.

Exemplary components, configurations and chimeric Tim4 receptor sequences of the present disclosure are provided in Table 1.

TABLE 1

| | | SEQ ID |
|---|---|---|
| Name | Amino Acid Sequence | NO: |
| Tim4 binding | MSKEPLILWLMIEFWWLYLTPVTSETVVTEVLGHRVT | 2 |
| domain | LPCLYSSWSHNSNSMCWGKDQCPYSGCKEALIRTDGM | |
| | RVTSRKSAKYRLQGTIPRGDVSLTILNPSESDSGVYCCR | |
| | IEVPGWFNDVKINVRLNLQRASTTTHRTATTTTRRTTT | |
| | TSPTTTRQMTTTPAALPTTVVTTPDLTTGTPLQMTTIAV | |
| | FTTANTCLSLTPSTLPEEATGLLTPEPSKEGPILTAESET | |
| | VLPSDSWSSVESTSADTVLLTSKESKVWDLPSTSHVSM | |
| | WKTSDSVSSPQPGASDTAVPEQNKTTKTGQMDGIPMS | |
| | MKNEMPISQ | |
| Modified IgG4 | ESKYGPPCPPCP | 3 |
| hinge | | |
| Wildtype | RSKRSRLLHSDYMNMTPRRPGPTRKHYQPYAPPRDFA | 118 |
| CD28 | AYRS | |
| costimulatory | | |
| signaling | | |
| domain | | |
| CD28 | RSKRSRGGHSDYMNMTPRRPGPTRKHYQPYAPPRDFA | 119 |
| costimulatory | AYRS | |
| signaling | | |
| domain with | | |
| L186G/L187G | | |
| substitutions | | |
| (positions | | |
| reference to | | |
| full length | | |
| protein) | | |
| OX40 | ALYLLRRDQRLPPDAHKPPGGGSFRTPIQEEQADAHST | 120 |
| costimulatory | LAKI | |
| signaling | | |
| domain | | |
| CD2 | KRKKQRSRRNDEELETRAHRVATEERGRKPHQIPASTP | 121 |
| costimulatory | QNPATSQHPPPPPGHRSQAPSHRPPPPGHRVQHQPQKR | |
| signaling | PPAPSGTQVHQQKGPPLPRPRVQPKPPHGAAENSLSPSS | |
| domain | N | |
| 4-1BB | KRGRKKLLYIFKQPFMRPVQTTQEEDGCSCRFPEEEEG | 122 |
| costimulatory | GCEL | |
| signaling | | |
| domain | | |
| CD27 | QRRKYRSNKGESPVEPAEPCHYSCPREEEGSTIPIQEDY | 123 |
| costimulatory | RKPEPACSP | |
| signaling | | |
| domain | | |
| ICAM-1 | NRQRKIKKYRLQQAQKGTPMKPNTQATPP | 124 |
| costimulatory | | |
| signaling | | |
| domain | | |
| LFA-1 | KALIHLSDLREYRRFEKEKLKSQWNNDNPLFKSATTTV | 125 |
| costimulatory | MNPKFAES | |
| signaling | | |
| domain | | |
| ICOS | CWLTKKKYSSSVHDPNGEYMEMRAVNTAKKSRLTDV | 126 |
| costimulatory | TL | |
| signaling | | |
| domain | | |
| CD30 | HRRACRKRIRQKLHLCYPVQTSQPKLELVDSRPRRSST | 127 |
| costimulatory | QLRSGASVTEPVAEERGLMSQPLMETCHSVGAAYLES | |
| signaling | LPLQDASPAGGPSSPRDLPEPRVSTEHTNNKIEKIYIMK | |
| domain | ADTVIVGTVKAELPEGRGLAGPAEPELEEELEADHTPH | |
| | YPEQETEPPLGSCSDVMLSVEEEGKEDPLPTAASGK | |
| CD40 | KKVAKKPTNKAPHPKQEPQEINFPDDLPGSNTAAPVQE | 128 |
| costimulatory | TLHGCQPVTQEDGKESRISVQERQ | |
| signaling | | |
| domain | | |
| PD-1 | CSRAARGTIGARRTGQPLKEDPSAVPVFSVDYGELDFQ | 129 |
| costimulatory | WREKTPEPPVPCVPEQTEYATIVFPSGMGTSSPARRGS | |
| signaling | ADGPRSAQPLRPEDGHCSWPL | |
| domain | | |
| CD7 | RTQIKKLCSWRDKNSAACVVYEDMSHSRCNTLSSPNQ | 130 |
| costimulatory | YQ | |
| signaling | | |
| domain | | |
| LIGHT | MEESVVRPSVFVVDGQTDIPFTRLGRSHRRQSCSVAR | 131 |
| costimulatory | | |
| signaling | | |
| domain | | |
| NKG2C | MSKQRGTFSEVSLAQDPKRQQRKPKGNKSSISGTEQEI | 132 |
| costimulatory | FQVELNLQNPSLNHQGIDKIYDCQGLLPPPEK | |
| signaling | | |
| domain | | |
| B7-H3 | CWRKIKQSCEEENAGAEDQDGEGEGSKTALQPLKHSD | 133 |
| costimulatory | SKEDDGQEIA | |
| signaling | | |
| domain | | |
| wildtype CD3ζ | RVKFSRSADAPAYQQGQNQLYNELNLGRREEYDVLD | 134 |
| activating | KRRGRDPEMGGKPQRRKNPQEGLYNELQKDKMAEAY | |
| domain | SEIGMKGERRRGKGHDGLYQGLSTATKDTYDALHMQ | |
| | ALPPR | |
| mutated human | RVKFSRSADAPAYQQGQNQLYNELNLGRREEYDVLD | 135 |
| CD3ζ ITAM- | KRRGRDPEMGGKPRRKNPQEGLYNELQKDKMAEAYS | |
| containing | EIGMKGERRRGKGHDGLYQGLSTATKDTYDALHMQA | |
| activating | LPPR | |
| domain | | |
| CD3γ ITAM- | GQDGVRQSRASDKQTLLPNDQLYQPLKDREDDQYSHL | 136 |
| containing | QGNQLRRN | |
| activating | | |
| domain | | |
| CD3δ ITAM- | GHETGRLSGAADTQALLRNDQVYQPLRDRDDAQYSH | 137 |
| containing | LGGNWARNK | |
| activating | | |
| domain | | |
| CD3ϵ ITAM- | KNRKAKAKPVTRGAGAGGRQRGQNKERPPPVPNPDY | 138 |
| containing | EPIRKGQRDLYSGLNQRRI | |
| activating | | |
| domain | | |
| CDS ITAM- | KKLVKKFRQKKQRQWIGPTGMNQNMSFHRNHTATVR | 139 |
| containing | SHAENPTASHVDNEYSQPPRNSHLSAYPALEGALHRSS | |
| activating | MQPDNSSDSDYDLHGAQRL | |
| domain | | |
| CD22 ITAM- | KLQRRWKRTQSQQGLQENSSGQSFFVRNKKVRRAPLS | 140 |
| containing | EGPHSLGCYNPMMEDGISYTTLRFPEMNIPRTGDAESS | |
| activating | EMQRPPPDCDDTVTYSALHKRQVGDYENVIPDFPEDE | |
| domain | GIHYSELIQFGVGERPQAQENVDYVILKH | |
| CD79a ITAM- | RKRWQNEKLGLDAGDEYEDENLYEGLNLDDCSMYED | 141 |
| containing | ISRGLQGTYQDVGSLNIGDVQLEKP | |
| activating | | |
| domain | | |
| DAP10 ITAM- | LCARPRRSPAQEDGKVYINMPGRG | 142 |
| containing | | |
| activating | | |
| domain | | |
| CD66d ITAM- | AKTGRTSIQRDLKEQQPQALAPGRGPSHSSAFSMSPLS | 143 |
| containing | TAQAPLPNPRTAASIYEELLKHDTNIYCRMDHKAEVAS | |
| activating | | |
| domain | | |
| Tim4 | LLMIIAPSLGFVLFALFVAFL | 144 |
| transmembrane | | |
| domain | | |
| CD28 | FWVLVVVGGVLACYSLLVTVAFIIFWV | 145 |
| transmembrane | | |
| domain | | |
| 4-1BB | IISFFLALTSTALLFLLFFLTLRFSVV | 146 |
| transmembrane | | |
| domain | | |
| OX40 | VAAILGLGLVLGLLGPLAILL | 147 |
| transmembrane | | |
| domain | | |
| CD27 | ILVIFSGMFLVFTLAGALFLH | 148 |
| transmembrane | | |
| domain | | |
| ICOS | FWLPIGCAAFVVVCILGCILI | 149 |
| transmembrane | | |
| domain | | |
| CD2 | IYLIIGICGGGSLLMVFVALLVFYIT | 150 |
| transmembrane | | |
| domain | | |
| LFA-1 | IAAIVGGTVAGIVLIGILLLVIW | 151 |
| transmembrane | | |
| domain | | |
| CD30 | PVLDAGPVLFWVILVLVVVVGSSAFLLC | 152 |
| transmembrane | | |
| domain | | |
| CD40 | ALVVIPIIFGILFAILLVLVFI | 153 |
| transmembrane | | |
| domain | | |
| PD-1 | VGVVGGLLGSLVLLVWVLAVI | 154 |
| transmembrane | | |
| domain | | |
| CD7 | AALAVISFLLGLGLGVACVLA | 155 |
| transmembrane | | |
| domain | | |
| LIGHT | VGLGLLLLLMGAGLAVQGWFL | 156 |
| transmembrane | | |
| domain | | |
| NKG2C | LTAEVLGIICIVLMATVLKTIVL | 157 |
| transmembrane | | |
| domain | | |
| B7-H3 | LWVTVGLSVCLIALLVALAFV | 158 |
| transmembrane | | |
| domain | | |
| Tim4 binding | MSKGLLLLWLVTELWWLYLTPAASEDTIIGFLGQPVTL | 159 |
| domain-CD28 | PCHYLSWSQSRNSMCWGKGSCPNSKCNAELLRTDGTR | |
| transmembrane | IISRKSTKYTLLGKVQFGEVSLTISNTNRGDSGVYCCRI | |
| -CD28 | EVPGWFNDVKKNVRLELRRATTTKKPTTTTRPTTTPY | |
| costimulatory | VTTTTPELLPTTVMTTSVLPTTTPPQTLATTAFSTAVTT | |
| signaling | CPSTTPGSFSQETTKGSAFTTESETLPASNHSQRSMMTI | |
| domain, amino | STDIAVLRPTGSNPGILPSTSQLTTQKTTLTTSESLQKTT | |
| acids 1-22 are | KSHQINSRQTFWVLVVVGGVLACYSLLVTVAFIIFWVR | |
| signal peptide | SKRSRGGHSDYMNMTPRRPGPTRKHYQPYAPPRDFAA | |
| | YRS | |
| Tim4 binding | MSKGLLLLWLVTELWWLYLTPAASEDTIIGFLGQPVTL | 160 |
| domain-Tim4 | PCHYLSWSQSRNSMCWGKGSCPNSKCNAELLRTDGTR | |
| transmembrane | IISRKSTKYTLLGKVQFGEVSLTISNTNRGDSGVYCCRI | |
| -CD28 | EVPGWENDVKKNVRLELRRATTTKKPTTTTRPTTTPY | |
| costimulatory | VTTTTPELLPTTVMTTSVLPTTTPPQTLATTAFSTAVTT | |
| signaling | CPSTTPGSFSQETTKGSAFTTESETLPASNHSQRSMMTI | |
| domain, amino | STDIAVLRPTGSNPGILPSTSQLTTQKTTLTTSESLQKTT | |
| acids 1-22 are | KSHQINSRQTILIIACCVGFVLMVLLFLAFLRSKRSRGG | |
| signal peptide | HSDYMNMTPRRPGPTRKHYQPYAPPRDFAAYRS | |
| Tim4 binding | MSKGLLLLWLVTELWWLYLTPAASEDTIIGFLGQPVTL | 161 |
| domain-Tim4 | PCHYLSWSQSRNSMCWGKGSCPNSKCNAELLRTDGTR | |
| transmembrane | IISRKSTKYTLLGKVQFGEVSLTISNTNRGDSGVYCCRI | |
| -4-1BB | EVPGWFNDVKKNVRLELRRATTTKKPTTTTRPTTTPY | |
| costimulatory | VTTTTPELLPTTVMTTSVLPTTTPPQTLATTAFSTAVTT | |
| signaling | CPSTTPGSFSQETTKGSAFTTESETLPASNHSQRSMMTI | |
| domain-CD36 | STDIAVLRPTGSNPGILPSTSQLTTQKTTLTTSESLQKTT | |
| ITAM | KSHQINSRQTILIIACCVGFVLMVLLFLAFLKRGRKKLL | |
| containing | YIFKQPFMRPVQTTQEEDGCSCRFPEEEEGGCELRVKF | |
| activating | SRSADAPAYQQGQNQLYNELNLGRREEYDVLDKRRG | |
| domain, amino | RDPEMGGKPRRKNPQEGLYNELQKDKMABAYSEIGM | |
| acids 1-22 are | KGERRRGKGHDGLYQGLSTATKDTYDALHMQALPPR | |
| signal peptide | | |
| Construct 13A | MSKEPLILWLMIEFWWLYLTPVTS | 162 |
| TIM4 binding | ETVVTEVLGHRVTLPCLYSSWSHNSNSMCWGKDQCP | |
| domain-CD28 | YSGCKEALIRTDGMRVTSRKSAKYRLQGTIPRGDVSLT | |
| TM-CD28 | ILNPSESDSGVYCCRIEVPGWFNDVKINVRLNLQRAST | |
| costim-CD3ζ | TTHRTATTTTRRTTTTSPTTTRQMTTTPAALPTTVVTTP | |
| (amino acids 1- | DLTTGTPLQMTTIAVFTTANTCLSLTPSTLPEEATGLLT | |
| 24 signal | PEPSKEGPILTAESETVLPSDSWSSVESTSADTVLLTSKE | |
| peptide) | SKVWDLPSTSHVSMWKTSDSVSSPQPGASDTAVPEQN | |
| | KTTKTGQMDGIPMSMKNEMPISQ | |
| | FWVLVVVGGVLACYSLLVTVAFIIFWV | |
| | RSKRSRLLHSDYMNMTPRRPGPTRKHYQPYAPPRDFAAYR | |
| | SSRVKFSRSADAPAYQQGQNQLYNELNLGRREEYDVL | |
| | DKRRGRDPEMGGKPQRRKNPQEGLYNELQKDKMAEA | |
| | YSEIGMKGERRRGKGHDGLYQGLSTATKDTYDALHM | |
| | QALPPR | |
| GM-CSF | MLLLVTSLLLCELPHPAFLLIP | 163 |
| signal peptide | | |
| Tim4 signal | MSKEPLILWLMIEFWWLYLTPVTS | 11 |
| peptide | | |
| Mouse Tim4 | ILIIACCVGFVLMVLLFLAFL | 23 |
| transmembrane | | |
| domain | | |
| Mouse Tim4 | MSKGLLLLWLVTELWWLYLTPAASEDTIIGFLGQPVTL | 24 |
| binding | PCHYLSWSQSRNSMCWGKGSCPNSKCNAELLRTDGTR | |
| domain, amino | IISRKSTKYTLLGKVQFGEVSLTISNTNRGDSGVYCCRI | |
| acids 1-22 are | EVPGWENDVKKNVRLELRRATTTKKPTTTTRPTTTPY | |
| signal peptide | VTTTTPELLPTTVMTTSVLPTTTPPQTLATTAFSTAVTT | |
| | CPSTTPGSFSQETTKGSAFTTESETLPASNHSQRSMMTI | |
| | STDIAVLRPTGSNPGILPSTSQLTTQKTTLTTSESLQKTT | |
| | KSHQINSRQT | |
| Mouse Tim4 | MSKGLLLLWLVTELWWLYLTPA | 25 |
| signal peptide | | |

Further embodiments of the chimeric Tim4 receptors described herein comprise a single chain chimeric protein, the single chain chimeric protein comprising: an extracellular domain comprising a Tim4 binding domain; an intracellular signaling domain comprising a first costimulatory signaling domain and an ITAM-containing activating domain, wherein the ITAM-containing activating domain comprises a DAP12 signaling domain; and a transmembrane domain positioned between and connecting the extracellular domain and the intracellular signaling domain.

A Tim4 binding domain suitable for use in a chimeric Tim4 receptor of the present disclosure may be any polypeptide or peptide derived from a Tim4 molecule that specifically binds phosphatidylserine. In certain embodiments, the Tim4 binding domain is derived from human Tim4. An exemplary human Tim4 molecule is provided in Uniprot. Ref. Q96H15 (SEQ ID NO:1). An exemplary human Tim4 binding domain comprises or consists of an amino acid sequence of SEQ ID NO:2 or amino acids 25-314 of SEQ ID NO:2. An exemplary mouse Tim4 binding domain comprises or consists of an amino acid sequence of SEQ ID NO:24 or amino acids 23-279 of SEQ ID NO:24. In certain embodiments, the Tim4 binding domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:2 or amino acids 25-314 of SEQ ID NO:2, or SEQ LD NO:24 or amino acids 23-279 of SEQ ID NO:24. In certain embodiments, the Tim4 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:2 or amino acids 25-3141 of SEQ ID NO:2, or SEQ ID NO:24 or amino acids 23-279 of SEQ ID NO:24.

In certain embodiments, the extracellular domain optionally comprises an extracellular, non-signaling spacer or linker domain. Where included, such a spacer or linker domain may position the binding domain away from the host cell surface to further enable proper cell/cell contact, binding, and activation. When included in a chimeric receptor as described herein, an extracellular spacer domain is generally located between the extracellular binding domain and the transmembrane domain of the chimeric Tim4 receptor. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG₄, IgA, IgD). An immunoglobulin hinge region may be a wild type immunoglobulin hinge region or an altered wild type immunoglobulin hinge region. An altered IgG4 hinge region is described in PCT Publication No. WO 2014/031687, which hinge region is incorporated herein by reference in its entirety. In a particular embodiment, an extracellular spacer domain comprises a modified IgG4 binge region having an amino acid sequence of ESKYGPPCPPCP (SEQ ID NO:3). Other examples of hinge regions that may be used in the chimeric Tim4 receptors described herein include the hinge region from the extracellular regions of type 1 membrane proteins, such as CD8a, CD4, CD28 and CD7, which may be wild-type or variants thereof. In some embodiments, an extracellular spacer domain comprises a CD28 hinge region having an amino acid sequence of SEQ ID NO:32. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

In certain embodiments, an extracellular domain comprises polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, an extracellular domain is murine, human, or chimeric.

In certain embodiments, an intracellular signaling domain comprises a costimulatory signaling domain and an ITAM-containing activating domain, wherein the ITAM-containing activating domain comprises DAP12 signaling domain. The costimulatory signaling domain may be any portion of a costimulatory signaling molecule that retains sufficient signaling activity. In some embodiments, a full length or full length intracellular component of a costimulatory signaling molecule is used. In some embodiments, a truncated portion of a costimulatory signaling molecule or intracellular component of a costimulatory signaling molecule is used, provided that the truncated portion retains sufficient signal transduction activity. In further embodiments, a costimulatory signaling domain is a variant of a whole or truncated portion of a costimulatory signaling molecule, provided that the variant retains sufficient signal transduction activity (i.e., is a functional variant).

In certain embodiments, the costimulatory signaling domain comprises a CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), PD-1, CD273 (PD-L2), CD274 (PD-L1), B7-13 (CD276), ICOS (CD278), DAP10, LAT, LFA-1 (CD11/CD18), LIGHT, NKG2C, SLP76, or TRIM signaling domain. In particular embodiments, the costimulatory signaling domain comprises an OX40, CD2, CD27, CD28, ICAM-1, LFA-1 (CD11a/CD18), ICOS (CD278), or 4-1BB (CD137) signaling domain. An exemplary CD28 costimulatory signaling domain comprises or consists of an amino acid sequence of SEQ ID NO: 164 or 165. An exemplary OX40 costimulatory signaling domain comprises or consists of an amino acid sequence of SEQ ID NO:166. An exemplary CD2 costimulatory signaling domain comprises or consists of an amino acid sequence of SEQ ID NO:167. An exemplary 4-1BB costimulatory signaling domain comprises or consists of an amino acid sequence of SEQ ID NO:168. An exemplary CD27 costimulatory signaling domain comprises or consists of an amino acid sequence of SEQ FD NO:169. An exemplary ICAM-1 costimulatory signaling domain comprises or consists of an amino acid sequence of SEQ ID NO:170. An exemplary LFA-1 costimulatory signaling domain comprises or consists of an amino acid sequence of SEQ ID NO:171. An exemplary ICOS costimulatory signaling domain comprises or consists of an amino acid sequence of SEQ ID NO:172. An exemplary CD30 costimulatory signaling domain comprises or consists of an amino acid sequence of SEQ ID NO:173. An exemplary CD40 costimulatory signaling domain comprises or consists of an amino acid sequence of SEQ ID NO:174. An exemplary PD-1 costimulatory signaling domain comprises or consists of an amino acid sequence of SEQ ID NO:175. An exemplary CD7 costimulatory signaling domain comprises or consists of an amino acid sequence of SEQ ID NO:176. An exemplary LIGHT costimulatory signaling domain comprises or consists of an amino acid sequence of SEQ ID NO:177. An exemplary NKG2C costimulatory signaling domain comprises or consists of an amino acid sequence of SEQ ID NO:178. An exemplary B7-H3 costimulatory signaling domain comprises or consists of an amino acid sequence of SEQ ID NO:179. In certain embodiments, the costimulatory signaling domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS: 164-179. In certain embodiments, the costimulatory signaling domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of any one of SEQ ID NOS: 164-179. In certain embodiments, the intracellular signaling comprises a second costimulatory signaling domain. In preferred embodiments, the first costimulatory signaling domain and second costimulatory signaling domain are different.

The DAP12 signaling domain may recapitulate TCR signaling independently of endogenous ICR complexes. In certain embodiments, signaling via the DAP12 signaling domain leads to mediation of a T cell response, including, but not limited to, proliferation, activation, differentiation, and the like. The DAP12 signaling domain may be any portion of the DAP12 molecule that retains sufficient signaling activity. In some embodiments, a full length or full length intracellular component of the DAP12 molecule is used. In some embodiments, a truncated portion of DAP12 or intracellular component of a DAP12 is used, provided that the truncated portion retains sufficient signal transduction activity. In further embodiments, the DAP12 signaling domain is a variant of a whole or truncated portion of DAP12, provided that the variant retains sufficient signal transduction activity (i.e., is a functional variant).

An exemplary human DAP12 molecule is provided in Uniprot. Ref. 043914. An exemplary DAP12 signaling domain comprises or consists of an amino acid sequence of SEQ ID NO: 180. In certain embodiments, the DAP12 signaling domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:180. In certain embodiments, the DAP12 signaling domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence to SEQ ID NO: 180.

In another embodiment, an intracellular signaling domain comprises a CD28 costimulatory signaling domain and a DAP12 signaling domain. In another embodiment, an intracellular signaling domain comprises a 4-1BB costimulatory signaling domain and a DAP12 signaling domain. In yet another embodiment, an intracellular signaling domain comprises a CD27 costimulatory signaling domain and a DAP12 signaling domain. In another embodiment, an intracellular signaling domain comprises a ICOS costimulatory signaling domain and a DAP12 signaling domain. In another embodiment, an intracellular signaling domain comprises a LFA-1 costimulatory signaling domain and a DAP12 signaling domain. In another embodiment, an intracellular signaling domain comprises an OX40 costimulatory signaling domain and a DAP12 signaling domain. In yet another embodiment, an intracellular signaling domain comprises a CD2 costimulatory signaling domain and a DAP12 signaling domain. In still another embodiment, an intracellular signaling domain comprises an ICAM-1 costimulatory signaling domain and a DAP12 signaling domain.

Intracellular signaling domains may be derived from a mammalian species, including humans, primates, cows, horses, goats, sheep, dogs, cats, mice, rats, rabbits, guinea pigs, pigs, and transgenic species thereof.

In certain embodiments, the transmembrane domain is derived from an integral membrane protein (e.g., receptor, cluster of differentiation (CD) molecule, enzyme, transporter, cell adhesion molecule, or the like). In one embodiment, the transmembrane domain is selected from the same molecule as the molecule from which the extracellular domain is derived. In another embodiment, the transmembrane domain is selected from the same molecule as the molecule from which the intracellular signaling domain is derived. For example, a chimeric Tim4 receptor may comprise a Tim4 binding domain and a Tim4 transmembrane domain. In another example, a chimeric Tim4 receptor may comprise a CD28 transmembrane domain and a CD28 costimulatory signaling domain. In certain embodiments, the transmembrane domain and the extracellular domain are derived from different molecules; the transmembrane domain and the intracellular signaling domain are derived from different molecules; or the transmembrane domain, extracellular domain, and intracellular signaling domain are all derived from different molecules. Examples of transmembrane domains that may be used in chimeric Tim4 receptors of the present disclosure include transmembrane domains from Tim4, CD3ζ, CD3γ, CD3δ, CD3ε, CD28, CD45, CD4, CD5, CD8, CD9, CD16, CD22, CD33, CD37, CD64, CD80, CD86, CD134, CD137, CD154, CD27, CD28, 4-1BB, OX40, CD30, CD40, PD-1, ICOS, LFA-1, CD2, CD⁷, LIGHT, NKG2C, and B7-H3. An exemplary Tim4 transmembrane domain comprises or consists of an amino acid sequence of SEQ ID NO: 144 or 23. An exemplary CD28 transmembrane domain comprises or consists of an amino acid sequence of SEQ ID NO: 181. An exemplary 4-1BB transmembrane domain comprises or consists of an amino acid sequence of SEQ ID NO:182. An exemplary OX40 transmembrane domain comprises or consists of an amino acid sequence of SEQ ID NO: 183. An exemplary CD27 transmembrane domain comprises or consists of an amino acid sequence of SEQ ID NO:184. An exemplary ICOS transmembrane domain comprises or consists of an amino acid sequence of SEQ ID NO:185. An exemplary CD2 transmembrane domain comprises or consists of an amino acid sequence of SEQ ID NO: 186. An exemplary LFA-1 transmembrane domain comprises or consists of an amino acid sequence of SEQ ID NO: 187. An exemplary CD30 transmembrane domain comprises or consists of an amino acid sequence of SEQ ID NO: 188. An exemplary CD40 transmembrane domain comprises or consists of an amino acid sequence of SEQ ID NO:189. An exemplary PD-1 transmembrane domain comprises or consists of an amino acid sequence of SEQ ID NO:190. An exemplary CD7 transmembrane domain comprises or consists of an amino acid sequence of SEQ ID NO: 191. An exemplary LIGHT transmembrane domain comprises or consists of an amino acid sequence of SEQ ID NO:192. An exemplary NKG2C transmembrane domain comprises or consists of an amino acid sequence of SEQ ID NO 193. An exemplary B7-H3 transmembrane domain comprises or consists of an amino acid sequence of SEQ ID NO: 194. In certain embodiments, the transmembrane domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:23, 144, and 181-194. In certain embodiments, the transmembrane domain comprises an amino acid sequence having at least about 1, 2, 3, 4, 5, 6, 7, 8, 9, or 10 amino acid modifications (e.g., deletion, additions, substitutions) to an amino acid sequence of any one of SEQ ID NOS:23, 144, and 181-194.

Transmembrane domains may derived from any mammalian species, including humans, primates, cows, horses, goats, sheep, dogs, cats, mice rats, rabbits, guinea pigs, pigs, and transgenic species thereof.

In certain embodiments, a chimeric Tim4 receptor comprises polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, a chimeric Tim4 receptor is murine, chimeric, human, or humanized.

It is understood that direct fusion of one domain to another domain of a chimeric Tim4 receptor described herein does not preclude the presence of intervening junction amino acids. Junction amino acids may be natural or non-natural (e.g. resulting from the construct design of a chimeric protein). For example, junction amino acids may result from restriction enzyme sites used for joining one domain to another domain or cloning polynucleotides encoding chimeric Tim4 receptors into vectors.

Exemplary components, configurations and chimeric Tim4 receptor sequences of the present disclosure are provided in Table 2.

TABLE 2

| | | SEQ |
|---|---|---|
| | | ID |
| Name | Amino Acid Sequence | NO: |
| human Tim4 | MSKEPLILWLMIEFWWLYLTPVTSETVVTEVLGHRVTLP | 2 |
| binding | CLYSSWSHNSNSMCWGKDQCPYSGCKEALIRTDGMRV | |
| domain (amino | TSRKSAKYRLQGTIPRGDVSLTILNPSESDSGVYCCRIEV | |
| acids 1-24 | PGWFNDVKINVRLNLQRASTTTHRTATTTTRRTTTTSPT | |
| signal peptide) | TTRQMTTTPAALPTTVVTTPDLTTGTPLQMTTIAVFTTA | |
| | NTCLSLTPSTLPEEATGLLTPEPSKEGPILTAESETVLPSDS | |
| | WSSVESTSADTVLLTSKESKVWDLPSTSHVSMWKTSDS | |
| | VSSPQPGASDTAVPEQNKTTKTGQMDGIPMSMKNEMPI | |
| | SQ | |
| mouse Tim4 | MSKGLLLLWLVTELWWLYLTPAASEDTIIGFLGQPVTLP | 24 |
| binding | CHYLSWSQSRNSMCWGKGSCPNSKCNAELLRTDGTRIIS | |
| domain (amino | RKSTKYTLLGKVQFGEVSLTISNTNRGDSGVYCCRIEVP | |
| acids 1-22 | GWENDVKKNVRLELRRATTTKKPTTTTRPTTTPYVTTTT | |
| signal peptide) | PELLPTTVMTTSVLPTTTPPQTLATTAFSTAVTTCPSTTPG | |
| | SFSQETTKGSAFTTESETLPASNHSQRSMMTISTDIAVLR | |
| | PTGSNPGILPSTSQLTTQKTTLTTSESLQKTTKSHQINSRQ | |
| | T | |
| Modified IgG4 | ESKYGPPCPPCP | 3 |
| hinge | | |
| Wildtype | RSKRSRLLHSDYMNMTPRRPGPTRKHYQPYAPPRDFAA | 164 |
| CD28 | YRS | |
| costimulatory | | |
| signaling | | |
| domain | | |
| CD28 | RSKRSRGGHSDYMNMTPRRPGPTRKHYQPYAPPRDFAA | 165 |
| costimulatory | YRS | |
| signaling | | |
| domain with | | |
| L186G/L187G | | |
| substitutions | | |
| (positions | | |
| reference to | | |
| full length | | |
| protein) | | |
| OX40 | ALYLLRRDQRLPPDAHKPPGGGSFRTPIQEEQADAHSTL | 166 |
| costimulatory | AKI | |
| signaling | | |
| domain | | |
| CD2 | KRKKQRSRRNDEELETRAHRVATEERGRKPHQIPASTPQ | 167 |
| costimulatory | NPATSQHPPPPPGHRSQAPSHRPPPPGHRVQHQPQKRPP | |
| signaling | APSGTQVHQQKGPPLPRPRVQPKPPHGAAENSLSPSSN | |
| domain | | |
| 4-1BB | KRGRKKLLYIFKQPFMRPVQTTQEEDGCCRFPEEEEGG | 168 |
| costimulatory | CEL | |
| signaling | | |
| domain | | |
| CD27 | QRRKYRSNKGESPVEPAEPCHYSCPREEEGSTIPIQEDYR | 169 |
| costimulatory | KPEPACSP | |
| signaling | | |
| domain | | |
| ICAM-1 | NRQRKIKKYRLQQAQKGTPMKPNTQATPP | 170 |
| costimulatory | | |
| signaling | | |
| domain | | |
| LFA-1 | KALIHLSDLREYRRFEKEKLKSQWNNDNPLFKSATTTV | 171 |
| costimulatory | MNPKFAES | |
| signaling | | |
| domain | | |
| ICOS | CWLTKKKYSSSVHDPNGEYMFMRAVNTAKKSRLTDVT | 172 |
| costimulatory | L | |
| signaling | | |
| domain | | |
| CD30 | HRRACRKRIRQKLHLCYPVQTSQPKLELVDSRPRRSSTQ | 173 |
| costimulatory | LRSGASVTEPVAEERGLMSQPLMETCHSVGAAYLESLPL | |
| signaling | QDASPAGGPSSPRDLPEPRVSTEHTNNKIEKIYIMKADTV | |
| domain | IVGTVKAELPEGRGLAGPAEPELEEELEADHTPHYPEQET | |
| | EPPLGSCSDVMLSVEEEGKEDPLPTAASGK | |
| CD40 | KKVAKKPTNKAPHPKQEPQEINFPDDLPGSNTAAPVQET | 174 |
| costimulatory | LHGCQPVTQEDGKESRISVQERQ | |
| signaling | | |
| domain | | |
| PD-1 | CSRAARGTIGARRTGQPLKEDPSAVPVFSVDYGELDFQ | 175 |
| costimulatory | WREKTPEPPVPCVPEQTEYATIVFPSGMGTSSPARRGSA | |
| signaling | DGPRSAQPLRPEDGHCSWPL | |
| domain | | |
| CD7 | RTQIKKLCSWRDKNSAACVVYEDMSHSRCNTLSSPNQY | 176 |
| costimulatory | Q | |
| signaling | | |
| domain | | |
| LIGHT | MEESVVRPSVFVVDGQTDIPFTRLGRSHRRQSCSVAR | 177 |
| costimulatory | | |
| signaling | | |
| domain | | |
| NKG2C | MSKQRGTFSEVSLAQDPKRQQRKPKGNKSSISGTEQEIF | 178 |
| costimulatory | QVELNLQNPSLNHQGIDKIYDCQGLLPPPEK | |
| signaling | | |
| domain | | |
| B7-H3 | CWRKIKQSCEEENAGAEDQDGEGEGSKTALQPLKHSDS | 179 |
| costimulatory | KEDDGQEIA | |
| signaling | | |
| domain | | |
| DAP12 | YFLGRLVPRGRGAAEAATRKQRITETESPYQELQGQRSD | 180 |
| | VYSDLNTQRPYYK | |
| Tim4 | LLMIIAPSLGFVLFALFVAFL | 144 |
| transmembrane | | |
| domain | | |
| CD28 | FWVLVVVGGVLACYSLLVTVAFIIFWV | 181 |
| transmembrane | | |
| domain | | |
| 4-1BB | IISFFLALTSTALLFLLFFLTLRFSVV | 182 |
| transmembrane | | |
| domain | | |
| OX40 | VAAILGLGLVLGLLGPLAILL | 183 |
| transmembrane | | |
| domain | | |
| CD27 | ILVIFSGMFLVFTLAGALFLH | 184 |
| transmembrane | | |
| domain | | |
| ICOS | FWLPIGCAAFVVVCILGCILI | 185 |
| transmembrane | | |
| domain | | |
| CD2 | IYLIIGICGGGSLLMVFVALLVFYIT | 186 |
| transmembrane | | |
| domain | | |
| LFA-1 | IAAIVGGTVAGIVLIGILLLVIW | 187 |
| transmembrane | | |
| domain | | |
| CD30 | PVLDAGPVLFWVILVLVVVVGSSAFLLC | 188 |
| transmembrane | | |
| domain | | |
| CD40 | ALVVIPIIFGILFAILLVLVFI | 189 |
| transmembrane | | |
| domain | | |
| PD-1 | VGVVGGLLGSLVLLVWVLAVI | 190 |
| transmembrane | | |
| domain | | |
| CD7 | AALAVISFLLGLGLGVACVLA | 191 |
| transmembrane | | |
| domain | | |
| LIGHT | VGLGLLLLLMGAGLAVQGWFL | 192 |
| transmembrane | | |
| domain | | |
| NKG2C | LTAEVLGIICIVLMATVLKTIVL | 193 |
| transmembrane | | |
| domain | | |
| B7-H3 | LWVTVGLSVCLIALLVALAFV | 194 |
| transmembrane | | |
| domain | | |
| Tim4 binding | MSKEPLILWLMIEFWWLYLTPVTSETVVTEVLGHRVTLP | 195 |
| domain-CD28 | CLYSSWSHNSNSMCWGKDQCPYSGCKEALIRTDGMRVT | |
| hinge-CD28 | SRKSAKYRLQGTIPRGDVSLTILNPSESDSGVYCCRIEV | |
| transmembrane | PGWFNDVKINVRLNLQRASTTTHRTATTTTRRTTTTSPT | |
| -CD28 | TTRQMTTTPAALPTTVVTTPDLTTGTPLQMTTIAVFTTA | |
| costimulatory | NTCLSLTPSTLPEEATGLLTPEPSKEGPILTAESETVLPS | |
| signaling | DSWSSVESTSADTVLLTSKESKVWDLPSTSHVSMWKTSDS | |
| domain- | VSSPQPGASDTAVPEQNKTTKTGQMDGIPMSMKNEMPISQ | |
| DAP12 | IEVMYPPPYLDNEKSNGTIIHVKGKHLCPSPLFPGPSKPF | |
| signaling | WVLVVVGGVLACYSLLVTVAFIIFWVRSKRSRLLHSDYM | |
| domain, amino | NMTPRRPGPTRKHYQPYAPPRDFAAYRSYFLGRLVPRGRG | |
| acids 1-24 are | AAEAATRKQRITETESPYQELQGQRSDVYSDLNTQRPYY | |
| signal peptide | K | |
| (CTX140) | | |
| Tim4 binding | MSKEPLILWLMIEFWWLYLTPVTSETVVTEVLGHRVTLP | 196 |
| domain- | CLYSSWSHNSNSMCWGKDQCPYSGCKEALIRTDGMRV | |
| modified IgG₄ | TSRKSAKYRLQGTIPRGDVSLTILNPSESDSGVYCCRIEV | |
| hinge-CD28 | PGWFNDVKINVRLNLQRASTTTHRTATTTTRRTTTTSPT | |
| transmembrane | TTRQMTTTPAALPTTVVTTPDLTTGTPLQMTTIAVFTTA | |
| -CD28 | NTCLSLTPSTLPEEATGLLTPEPSKEGPILTAESETVLPS | |
| costimulatory | DSWSSVESTSADTVLLTSKESKVWDLPSTSHVSMWKTSDS | |
| signaling | VSSPQPGASDTAVPEQNKTTKTGQMDGIPMSMKNEMPI | |
| domain- | SQESKYGPPCPPCPFWVLVVVGGVLACYSLLVTVAFIIF | |
| DAP12 | WVRSKRSRLLHSDYMNMTPRRPGPTRKHYQPYAPPRDFAA | |
| signaling | YRSYFLGRLVPRGRGAAEAATRKQRITETESPYQELQGQ | |
| domain, amino | RSDVYSDLNTQRPYYK | |
| acids 1-24 are | | |
| signal peptide | | |
| (CTX143) | | |
| Tim4 binding | MSKEPLILWLMIEFWWLYLTPVTSETVVTEVLGHRVTLP | 197 |
| domain-CD28 | CLYSSWSHNSNSMCWGKDQCPYSGCKEALIRTDGMRV | |
| transmembrane | | |
| -CD28 | | |
| costimulatory | | |
| signaling | | |
| domain- | | |
| DAP12 | TSRKSAKYRLQGTIPRGDVSLTILNPSESDSGVYCCRIEV | |
| signaling | PGWFNDVKINVRLNLQRASTTTHRTATTTTRRTTTTSPT | |
| domain, amino | TTRQMTTTPAALPTTVVTTPDLTTGTPLQMTTIAVFTTA | |
| acids 1-24 are | NTCLSLTPSTLPEEATGLLTPEPSKEGPILTAESETVLPS | |
| signal peptide | DSWSSVESTSADTVLLTSKESKVWDLPSTSHVSMWKTSDS | |
| (CTX137) | VSSPQPGASDTAVPEQNKTTKTGQMDGIPMSMKNEMPI | |
| | SQFWVLVVVGGVLACYSLLVTVAFIIFWVRSKRSRLLHSD | |
| | YMNMTPRRPGPTRKHYQPYAPPRDFAAYRSYFLGRLVPRG | |
| | RGAAEAATRKQRITETESPYQELQGQRSDVYSDLNTQRP | |
| | YYK | |
| Tim4 binding | MSKEPLILWLMIEFWWLYLTPVTSETVVTEVLGHRVTLP | 198 |
| domain-Tim4 | CLYSSWSHNSNSMCWGKDQCPYSGCKEALIRTDGMRVT | |
| TM-CD28- | SRKSAKYRLQGTIPRGDVSLTILNPSESDSGVYCCRIEV | |
| DAP12; amino | PGWFNDVKINVRLNLQRASTTTHRTATTTTRRTTTTSPT | |
| acids 1-24 are | TTRQMTTTPAALPTTVVTTPDLTTGTPLQMTTIAVFTTA | |
| signal peptide | NTCLSLTPSTLPEEATGLLTPEPSKEGPILTAESETVLPS | |
| (CTX256) | DSWSSVESTSADTVLLTSKESKVWDLPSTSHVSMWKTSDS | |
| | VSSPQPGASDTAVPEQNKTTKTGQMDGIPMSMKNEMPIS | |
| | QLLMIIAPSLGFVLFALFVAFLRSKRSRLLHSDYMNMTP | |
| | RRPGPTRKHYQPYAPPRDFAAYRSYFLGRLVPRGRGAA | |
| | EAATRKQRITETESPYQELQGQRSDVYSDLNTQRPYYK | |

In embodiments, a chimeric Tim receptor comprises a single chain chimeric protein, the single chain chimeric protein comprising: (a) an extracellular domain comprising a binding domain comprising: (i) a Tim1 IgV domain or Tim4 IgV domain and (ii) a Tim1 mucin domain or Tim4 mucin domain; (b) an intracellular signaling domain, wherein the intracellular signaling domain comprises a primary intracellular signaling domain and optionally a secondary intracellular signaling domain; and (c) a transmembrane domain positioned between and connecting the extracellular domain and the intracellular signaling domain.

In particular embodiments, the binding domain comprises: (i) a Tim1 IgV domain and a Tim1 mucin domain; (ii) a Tim4 IgV domain and a Tim4 mucin domain; (iii) a Tim1 IgV domain and a Tim4 mucin domain; or (iv) a Tim4 IgV domain and a Tim1 mucin domain. In certain embodiments, the extracellular domain of the chimeric Tim receptors described herein optionally includes an extracellular spacer domain positioned between and connecting the binding domain and transmembrane domain.

When expressed in a host cell, chimeric Tim receptors of the present disclosure can confer a phosphatidylserine-specific, cytotoxic phenotype to the modified host cell (e.g., the host cell becomes cytotoxic to a stressed, damaged, injured, apoptotic, or necrotic cell expressing phosphatidylserine on its surface). In certain embodiments, the chimeric Tim receptors induce apoptosis in targeted cells via release of granzymes, perforin, granulysin, or any combination thereof. In further embodiments, cells expressing a chimeric Tim receptor according to the present description exhibit an engulfment phenotype specific to phosphatidylserine presenting cells.

The intracellular signaling domain can include one or more effector (also referred to as “costimulatory signaling”) domains that costimulate the modified host cell. Signaling by the intracellular signaling domain(s) is triggered by binding of the extracellular domain to phosphatidylserine. In certain embodiments, the intracellular signaling domain comprises a first intracellular signaling domain. In further embodiments, the intracellular signaling domain comprises a first intracellular signaling domain and a second intracellular signaling domain. In some embodiments, the intracellular signaling domain comprises a first intracellular signaling domain, a second intracellular signaling domain, and a third intracellular signaling domain. Chimeric Tim receptors according to the present disclosure can be used in a variety of therapeutic methods where clearance of apoptotic, necrotic, damaged, or stressed cells is beneficial, while providing costimulation that enhances cellular immune response, reduces immune cell exhaustion, or both.

As described herein, a chimeric Tim receptor comprises an extracellular domain comprising a Tim binding domain. The Tim binding domain confers specificity to phosphatidylserine (PtdSer), which is a phospholipid with a negatively charged head-group and a component of the cell membrane. In healthy cells, phosphatidylserine is preferentially found in the inner leaflet of the cell membrane. However, when cells are stressed, damaged or undergo apoptosis or necrosis, phosphatidylserine is exposed on the outer leaflet of the cell membrane. Thus, phosphatidylserine may be used as a marker to distinguish stressed, damaged, apoptotic, necrotic, pyroptotic, or oncotic cells. Binding of phosphatidylserine by the Tim binding domain may block the interaction between the phosphatidylserine and another molecule and, for example, interfere with, reduce or eliminate certain functions of the phosphatidylserine (e.g., signal transduction). In some embodiments, the binding of a phosphatidylserine may induce certain biological pathways or identify the phosphatidylserine molecule or a cell expressing phosphatidylserine for elimination.

A Tim binding domain suitable for use in a chimeric Tim receptor of the present disclosure may be any polypeptide or peptide derived from a Tim1 and/or Tim4 molecule that specifically binds phosphatidylserine. In embodiments, a Tim binding domain comprises an IgV domain from Tim1 or Tim4, and a mucin domain from Tim1 or Tim4. For example, a Tim binding domain may comprise a Tim1 IgV domain and a Tim1 mucin domain. In another example, a Tim binding domain may comprise a Tim1 IgV domain and a Tim4 mucin domain. In another example, a Tim binding domain may comprise a Tim4 IgV domain and a Tim1 mucin domain. In another example, a Tim binding domain may comprise a Tim4 IgV domain and a Tim4 mucin domain.

Phosphatidylserine binding is generally regulated by the IgV domain. The core phosphatidylserine binding domain is a four amino acid sequence in the IgV domain (e.g., amino acids 95-98 of SEQ ID NO:34 or amino acids 92-95 of SEQ ID NO:38). A Tim4 binding domain binds minimally to cells with low phosphatidylserine density. A Tim1 binding domain binds more strongly to a lower phosphatidylserine density, resulting in a lower threshold for response. A summary of Tim1 and Tim4 binding to phosphatidylserine is provided in Table 3. By combining Tim1 IgV domain and Tim4 mucin domain, or Tim4 IgV domain and Tim1 mucin domain, the binding affinity of the binding domain to phosphatidylserine can be modulated. Further, such a combination in the Tim binding domain also provides a combination of the sensitivity to phosphatidylserine of Tim4 and the stability in protein expression of Tim1.

TABLE 3

| | | | | Co- |
|---|---|---|---|---|
| | Low PtdSer | Moderate PtdSer | High PtdSer | operativity |
| Tim-1 | Moderate | Strong | Strong | |
| | binding | binding | binding | |
| Tim-4 | Minimal | Moderate | Strong | Yes |
| | binding | binding | binding | |

Additionally, an RGD domain (e.g., amino acids 68-70 of SEQ ID NO:34) in an IgV domain may regulate integrin binding as a co-receptor for engulfment.

In certain embodiments, the Tim binding domain is derived from human Tim1 and/or Tim4. An exemplary human Tim1 molecule is provided in Uniprot. Ref. Q96D42 (SEQ ID NO:36). An exemplary human Tim1 binding domain comprises or consists of an amino acid sequence of SEQ ID NO:37 or SEQ ID NO:43. In certain embodiments, the Tim1 binding domain comprises or consists of an amino acid sequence having at least about 75%, 80%, 85%, 90%, 91%, 92% 93%, 94%, 95%, 96%, 97%, 98%, 99%, 99.5%, or 100% identity to SEQ ID NO:37 or SEQ ID NO:43. In certain embodiments, the Tim1 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:37 or SEQ ID NO:43.

An exemplary human Tim4 molecule is provided in Uniprot. Ref. Q96H15 (SEQ ID NO:1). An exemplary human Tim4 binding domain comprises or consists of an amino acid sequence of SEQ ID NO:2 or SEQ ID NO:42. An exemplary mouse Tim4 binding domain comprises or consists of an amino acid sequence of SEQ ID NO:24 or amino acids 23-279 of SEQ ID NO:24. In certain embodiments, the Tim4 binding domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:2 or SEQ ID NO:42, or SEQ ID NO:241 or amino acids 23-279 of SEQ ID NO:24. In certain embodiments, the Tim4 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:2 or SEQ ID NO:42, or SEQ ID NO:24 or amino acids 23-279 of SEQ ID NO:24.

In embodiments, the Tim binding domain comprises an IgV domain from Tim1. An exemplary human Tim1 IgV domain is provided in SEQ ID NO:38. In some embodiments, the Tim1 IgV domain is a modified Tim1 IgV domain comprising a R66G substitution in SEQ ID NO:38. The R66G substitution (e.g., amino acids 68-70 of SEQ ID NO:34) confers a RGD domain in Tim1 IgV domain, which may regulate integrin binding as a co-receptor for engulfment. In particular embodiments, the modified Tim1 IgV domain comprises or consists of the amino acid sequence of SEQ ID NO:41. In some embodiments, this modified Tim1 domain may increase phagocytic activity while preserving Tim1 sensitivity. In certain embodiments, the Tim1 IgV domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:38, SEQ ID NO:38 with a R66G substitution, or SEQ ID NO:41. In certain embodiments, the Tim1 IgV comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:38, SEQ ID NO:38 with a R66G substitution, or SEQ ID NO:41.

In other embodiments, the Tim binding domain comprises an IgV domain from Tim4. An exemplary human Tim4 IgV domain is provided in SEQ ID NO:34. In certain embodiments, the Tim4 IgV domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:34. In certain embodiments, the Tim4 IgV domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:34.

In embodiments, the Tim binding domain comprises a mucin domain from Tim1. An exemplary human Tim1 mucin domain is provided in SEQ ID NO:39. In certain embodiments, the Tim1 mucin domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:39. In certain embodiments, the Tim1 mucin domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:39.

In other embodiments, the Tim binding domain comprises a mucin domain from Tim4. An exemplary human Tim4 mucin domain is provided in SEQ ID NO:35. In certain embodiments, the Tim4 mucin domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:35. In certain embodiments, the Tim4 mucin domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:35.

In some embodiments, the Tim binding domain comprises a Tim1 IgV domain and a Tim1 mucin domain. In a particular embodiment, the Tim1 IgV domain comprises the amino acid sequence set forth in SEQ ID NO:38 and the Tim1 mucin domain comprises the amino acid sequence set forth in SEQ ID NO:39. In another particular embodiment, the Tim IgV domain comprises the amino acid sequence set forth in SEQ ID NO:38 with a R66G substitution and the Tim1 mucin domain comprises the amino acid sequence set forth in SEQ ID NO:39. In a further particular embodiment, the Tim1 IgV domain comprises the amino acid sequence set forth in SEQ ID NO:41 and the Tim1 mucin domain comprises the amino acid sequence set forth in SEQ ID NO:39. In some embodiments, the Tim1 IgV domain and Tim1 mucin domain together comprise or consist of the amino acid sequence set forth in SEQ ID NO:37 or SEQ ID NO:43.

In some embodiments, the Tim binding domain comprises a Tim4 IgV domain and a Tim4 mucin domain. In a particular embodiment, the Tim4 IgV domain comprises the amino acid sequence set forth in SEQ ID NO:34 and the Tim4 mucin domain comprises the amino acid sequence set forth in SEQ ID NO:35. In some embodiments, the Tim4 IgV domain and Tim4 mucin domain together comprise or consist of the amino acid sequence set forth in SEQ ID NO:2 or SEQ ID NO:42.

In some embodiments, the Tim binding domain comprises a Tim1 IgV domain and a Tim4 mucin domain. In a particular embodiment, the Tim1 IgV domain comprises the amino acid sequence set forth in SEQ ID NO:38 and the Tim4 mucin domain comprises the amino acid sequence set forth in SEQ ID NO:35. In another particular embodiment, the Tim1 IgV domain comprises the amino acid sequence set forth in SEQ ID NO:38 with a R66G substitution and the Tim4 mucin domain comprises the amino acid sequence set forth in SEQ ID NO:35. In a further particular embodiment, the Tim1 IgV domain comprises the amino acid sequence set forth in SEQ ID NO:41 and the Tim4 mucin domain comprises the amino acid sequence set forth in SEQ ID NO:35. In some embodiments, the Tim1 IgV domain further comprises the Tim1 signal sequence of SEQ ID NO:40.

In still further embodiments, the Tim binding domain comprises a Tim4 IgV domain and a Tim1 mucin domain. In a particular embodiment, the Tim4 IgV domain comprises the amino acid sequence set forth in SEQ ID NO:34 and the Tim1 mucin domain comprises the amino acid sequence set forth in SEQ ID NO:39. In some embodiments, the Tim4 IgV domain further comprises the Tim4 signal sequence of SEQ ID NO:11.

In certain embodiments, the extracellular domain optionally comprises an extracellular, non-signaling spacer or linker domain. Where included, such a spacer or linker domain may position the binding domain away from the host cell surface to further enable proper cell/cell contact, binding, and activation. When included in a chimeric receptor as described herein, an extracellular spacer domain is generally located between the extracellular binding domain and the transmembrane domain of the chimeric Tim receptor. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region.

An altered IgG4 hinge region is described in PCT Publication No. WO 2014/031687, which hinge region is incorporated herein by reference in its entirety. In a particular embodiment, an extracellular spacer domain comprises a modified IgG₄ hinge region having an amino acid sequence of ESKYGPPCPPCP (SEQ ID NO:3). Other examples of hinge regions that may be used in the chimeric Tim receptors described herein include the hinge region from the extracellular regions of type 1 membrane proteins, such as CD8a, CD4, CD28 and CD7, which may be wild-type or variants thereof. In a particular embodiment, an extracellular spacer domain comprises a CD28 hinge region having an amino acid sequence of SEQ ID NO:32. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

In certain embodiments, an extracellular domain comprises an amino acid sequences derived from any mammalian species, including humans, primates, cows, horses, goats, sheep, dogs, cats, mice, rats, rabbits, guinea pigs, pigs, transgenic species thereof, or any combination thereof. In certain embodiments, an extracellular domain is murine, human, or chimeric.

The intracellular signaling domain of a chimeric Tim receptor as described herein is an intracellular effector domain and is capable of transmitting functional signals to a cell in response to binding of the extracellular domain of the chimeric Tim receptor and phosphatidylserine. The signals transduced by the intracellular signaling domain promote effector function of the chimeric Tim receptor containing cell. Examples of effector function include cytotoxic activity, secretion of cytokines, proliferation, anti-apoptotic signaling, persistence, expansion, engulfment of a target cell or particle expressing phosphatidylserine on its surface, antigen capture, antigen processing, antigen presentation, or any combination thereof.

An intracellular signaling domain comprises a primary intracellular signaling domain. In some embodiments, an intracellular signaling domain comprises a primary intracellular signaling domain and a secondary intracellular signaling domain. In some embodiments, an intracellular signaling domain comprises a primary intracellular signaling domain, a secondary intracellular signaling domain, and a tertiary intracellular signaling domain. The primary, secondary, and/or tertiary intracellular signaling domains may independently be any portion of a signaling molecule that retains sufficient signaling activity. In some embodiments, a full length signaling molecule or full length intracellular component of a signaling molecule is used. In some embodiments, a truncated portion of a signaling molecule or intracellular component of a signaling molecule is used, provided that the truncated portion retains sufficient signal transduction activity. In some embodiments, a signaling domain is a variant of a whole or truncated portion of a signaling molecule, provided that the variant retains sufficient signal transduction activity (i.e., is a functional variant).

In some embodiments, the primary intracellular signaling domain comprises a Tim1 signaling domain, a Tim4 signaling domain, a TRAF2 signaling domain, a TRAF6 signaling domain, a CD28 signaling domain, a DAP12 signaling domain, a CD3ζ signaling domain, 4-1BB signaling domain, TLR2 signaling domain, or a TLR8 signaling domain.

In embodiments, the secondary intracellular signaling domain comprises a Tim1 signaling domain, a Tim4 signaling domain, a TRAF2 signaling domain, a TRAF6 signaling domain, a CD28 signaling domain, a DAP12 signaling domain, a CD3ζ signaling domain, 4-1BB signaling domain, TLR2 signaling domain, or a TLR8 signaling domain.

In some embodiments, the tertiary intracellular signaling domain comprises a Tim1 signaling domain, a Tim4 signaling domain, a TRAF2 signaling domain, a TRAF6 signaling domain, a CD28 signaling domain, a DAP12 signaling domain, a CD3ζ signaling domain, 4-1BB signaling domain, TLR2 signaling domain or a TLR8 signaling domain.

In some embodiments, the primary intracellular signaling domain comprises an immunoreceptor tyrosine-based activation motif (ITAM) containing signaling domain; the secondary intracellular signaling domain comprises a costimulatory signaling domain, Tim1 signaling domain or Tim4 signaling domain; and the tertiary intracellular signaling domain comprises a TLR signaling domain. An ITAM containing signaling domain generally contains at least one (one, two, three, four, or more) ITAMs, which refer to a conserved motif of YXXL/I-X₆₋₈-YXXL/I. An ITAM containing signaling domain may initiate T cell activation signaling following antigen binding or ligand engagement. ITAM-signaling domains include, for example, intracellular signaling domains of CD3γ, CD3δ, CD3ε, CD3ζ, CD5, C122, CD79a, CD278 (ICOS), DAP12, FcRγ, and CD66d. A costimulatory signaling domain, which, when activated in conjunction with a primary or classic (e.g., ITAM-driven) activation signal, promotes or enhances T cell response, such as T cell activation, cytokine production, proliferation, differentiation, survival, effector function, or combinations thereof. Costimulatory signaling domains for use in chimeric Tim receptors include, for example, CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, NKG2C, SLP76, TRIM, ZAP70, or any combination thereof. In some embodiments, the costimulatory signaling domain comprises a OX40, CD2, CD27, CD28, ICAM-1, LFA-1 (CD11a/CD18), ICOS (CD278), or 4-1BB (CD137) signaling domain. A TLR signaling domain may be a TLR1, TLR2, TLR3, TLR4, TLR5, TLR6, TLR7, TLR8, or TLR9 signaling domain. In some embodiments, the TLR signaling domain is a TLR2 signaling domain or TLR8 signaling domain.

As used herein, the designation of primary, secondary, and tertiary intracellular signaling domains includes but is not limited to arrangements of the primary intracellular signaling domain at the N-terminus, secondary intracellular signaling domain in the middle, and tertiary intracellular signaling domain at the C-terminus of the intracellular portion of the chimeric Tim receptor. Thus, designation of the primary intracellular signaling domain does not limit the use of the selected intracellular signaling domain at the N-terminus of the intracellular portion of the chimeric Tim receptor. Designation of the secondary intracellular signaling domain does not limit the use of the selected intracellular signaling domain in the middle (or at the C-terminus for those chimeric Tim receptors only having two intracellular signaling domains) of the intracellular portion of the chimeric Tim receptor. Designation of the tertiary intracellular signaling domain does not limit the use of the selected intracellular signaling domain at the C-terminus of the intracellular portion of the chimeric Tim receptor. Thus, different arrangements of the primary, secondary, and/or tertiary intracellular signaling domains within the intracellular portion of the chimeric Tim receptor are contemplated.

An exemplary Tim1 signaling domain comprises or consists of an amino acid sequence of SEQ ID NO:44. An exemplary Tim4 signaling domain comprises or consists of an amino acid sequence of SEQ ID NO:45, SEQ ID NO:224, or SEQ ID NO:225. An exemplary TRAF2 signaling domain comprises or consists of an amino acid sequence of SEQ ID NO:48. An exemplary TRAF6 signaling domain comprises or consists of an amino acid sequence of SEQ ID NO:46. An exemplary CD28 signaling domain comprises or consists of an amino acid sequence of SEQ ID NO:4 or 26. An exemplary DAP12 signaling domain comprises or consists of an amino acid sequence of SEQ ID NO:9. An exemplary CD3ζ signaling domain comprises or consists of an amino acid sequence of SEQ ID NO:27 or 5. An exemplary 4-1BB signaling domain comprises or consists of the amino acid sequence of SEQ ID NO:122. An exemplary TLR2 signaling domain comprises or consists of the amino acid sequence of SEQ ID NO:222. An exemplary TLR8 signaling domain comprises or consists of an amino acid sequence of SEQ ID NO:47.

In a particular embodiment, the Tim1 signaling domain comprises the amino acid sequence set forth in SEQ ID NO:44. In another particular embodiment, the Tim4 signaling domain comprises the amino acid sequence set forth in SEQ ID NO:45 SEQ ID NO:224, or SEQ ID NO:225. In another particular embodiment, the TRAF2 signaling domain comprises the amino acid sequence set forth in SEQ ID NO:48. In another particular embodiment, the TRAF6 signaling domain comprises the amino acid sequence set forth in SEQ ID NO:46. In another particular embodiment, the CD28 signaling domain comprises the amino acid sequence set forth in SEQ ID NO:4. In another particular embodiment, the CD28 signaling domain comprises the amino acid sequence set forth in SEQ ID NO:26. In another particular embodiment, the DAP12 signaling domain comprises the amino acid sequence set forth in SEQ ID NO:9. In another particular embodiment, the CD3 signaling domain comprises the amino acid sequence set forth in SEQ ID NO:27. In another particular embodiment, the CD3ζ signaling domain comprises the amino acid sequence set forth in SEQ ID NO:5. In another particular embodiment, the 4-1BB signaling domain comprises the amino acid sequence of SEQ ID NO: 122. In another particular embodiment, the TLR2 signaling domain comprises of the amino acid sequence of SEQ ID NO:222. In another particular embodiment, or the TLR8 signaling domain comprises the amino acid sequence set forth in SEQ ID NO:47.

In embodiments, the primary, secondary, and/or tertiary signaling domain (if present) comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:4, 5, 9, 26, 27, or 44-48. In certain embodiments, the primary, secondary, and/or tertiary signaling domains comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of any one of SEQ ID NOS:4, 5, 9, 26, 27, 44-48, 122, and 222. In embodiments, the primary signaling domain, secondary, and/or tertiary intracellular signaling domain are the same. In some embodiments, two or three of the primary, secondary, and tertiary intracellular signaling domains are different.

In certain embodiments, an intracellular signaling domain comprises a Tim1 intracellular signaling domain. In some embodiments, an intracellular signaling domain comprises a Tim4 intracellular signaling domain. In some embodiments, an intracellular signaling domain comprises a CD3ζ intracellular signaling domain. In some embodiments, an intracellular signaling domain comprises a CD28 intracellular signaling domain. In some embodiments, an intracellular signaling domain comprises a TRAF6 intracellular signaling domain. In some embodiments, an intracellular signaling domain comprises a TRAF2 intracellular signaling domain. In some embodiments, an intracellular signaling domain comprises a TLR2 intracellular signaling domain. In some embodiments, an intracellular signaling domain comprises a TLR8 intracellular signaling domain.

In certain embodiments, an intracellular signaling domain comprises a Tim1 primary intracellular signaling domain and a CD3ζ secondary intracellular signaling domain. In other embodiments, an intracellular signaling domain comprises a Tim4 primary intracellular signaling domain and a CD3ζ secondary intracellular signaling domain. In other embodiments, an intracellular signaling domain comprises a TLR8 primary intracellular signaling domain and a CD3ζ secondary intracellular signaling domain. In other embodiments, an intracellular signaling domain comprises a TLR2 primary intracellular signaling domain and a CD3ζ secondary intracellular signaling domain. In other embodiments, an intracellular signaling domain comprises a CD28 primary intracellular signaling domain and a DAP12 secondary intracellular signaling domain. In some embodiments, an intracellular signaling domain comprises a CD28 primary intracellular signaling domain and a CD3ζ secondary intracellular signaling domain. In some embodiments, an intracellular signaling domain comprises a CD28 primary intracellular signaling domain, a TLR2 secondary intracellular signaling domain, and a CD3ζ tertiary intracellular signaling domain. In some embodiments, an intracellular signaling domain comprises a CD28 primary intracellular signaling domain, a CD3ζ secondary intracellular signaling domain, and a TLR2 tertiary intracellular signaling domain. In some embodiments, an intracellular signaling domain comprises a CD28 primary intracellular signaling domain, a TLR8 secondary intracellular signaling domain, and a CD3ζ tertiary intracellular signaling domain. In some embodiments, an intracellular signaling domain comprises a CD28 primary intracellular signaling domain, a CD3ζ secondary intracellular signaling domain, and a TLR8 tertiary intracellular signaling domain. In some embodiments, an intracellular signaling domain comprises a TLR2 primary intracellular signaling domain and a CD3ζ secondary intracellular signaling domain. In some embodiments, an intracellular signaling domain comprises a CD3ζ primary intracellular signaling domain and a TLR2 secondary intracellular signaling domain. In some embodiments, an intracellular signaling domain comprises a TLR8 primary intracellular signaling domain and a CD3ζ secondary intracellular signaling domain. In some embodiments, an intracellular signaling domain comprises a CD3ζ primary intracellular signaling domain and a TLR8 secondary intracellular signaling domain. In some embodiments, an intracellular signaling domain comprises a TRAF6 primary intracellular signaling domain and a CD3ζ secondary intracellular signaling domain. In some embodiments, an intracellular signaling domain comprises a CD3ζ primary intracellular signaling domain and a TRAF6 secondary intracellular signaling domain. In some embodiments, an intracellular signaling domain comprises a CD28 primary intracellular signaling domain and a CD3ζ secondary intracellular signaling domain. In some embodiments, an intracellular signaling domain comprises a CD28 primary intracellular signaling domain, a TLR2 secondary intracellular signaling domain, and a CD3ζ tertiary intracellular signaling domain. In some embodiments, an intracellular signaling domain comprises a CD28 primary intracellular signaling domain, a CD3ζ secondary intracellular signaling domain, and a TLR2 tertiary intracellular signaling domain. In some embodiments, an intracellular signaling domain comprises a CD28 primary intracellular signaling domain, a TLR8 secondary intracellular signaling domain, and a CD3ζ tertiary intracellular signaling domain. In some embodiments, an intracellular signaling domain comprises a CD28 primary intracellular signaling domain, a CD3ζ secondary intracellular signaling domain, and a TLR8 tertiary intracellular signaling domain. In some embodiments, an intercellular signaling domain comprises a CD3ζ primary intracellular signaling domain and a TLR2 secondary intracellular signaling domain. In some embodiments, an intercellular signaling domain comprises a TLR2 primary intracellular signaling domain and a CD3ζ secondary intracellular signaling domain. In some embodiments, an intercellular signaling domain comprises a CD3ζ primary intracellular signaling domain and a TLR8 secondary intracellular signaling domain. In some embodiments, an intercellular signaling domain comprises a TLR8 primary intracellular signaling domain and a CD3ζ secondary intracellular signaling domain. In some embodiments, an intercellular signaling domain comprises a CD3ζ primary intracellular signaling domain and a TRAF6 secondary intracellular signaling domain. In some embodiments, an intercellular signaling domain comprises a TRAF6 primary intracellular signaling domain and a CD3ζ secondary intracellular signaling domain. In some embodiments, an intracellular signaling domain comprises a Tim4 primary intracellular signaling domain, a TLR2 secondary intracellular signaling domain, and a CD3ζ tertiary intracellular signaling domain. In some embodiments, an intracellular signaling domain comprises a Tim4 primary intracellular signaling domain, a CD3ζ secondary intracellular signaling domain, and a TLR2 tertiary intracellular signaling domain. In some embodiments, an intracellular signaling domain comprises a Tim4 primary intracellular signaling domain, a TLR8 secondary intracellular signaling domain, and a CD3ζ tertiary intracellular signaling domain. In some embodiments, an intracellular signaling domain comprises a Tim4 primary intracellular signaling domain, a CD3ζ secondary intracellular signaling domain, and a TLR8 tertiary intracellular signaling domain.

In a particular embodiment, an intracellular signaling domain comprises a Tim1 primary intracellular signaling domain comprising an amino acid sequence of SEQ ID NO:44 and a CD3; secondary intracellular signaling domain comprising an amino acid sequence of SEQ ID NO:27 or 5. In another particular embodiment, an intracellular signaling domain comprises a Tim4 primary intracellular signaling domain comprising an amino acid sequence of SEQ ID NO:45, SEQ ID NO:224, or SEQ ID NO:225, and a CD3ζ secondary intracellular signaling domain comprising an amino acid sequence of SEQ ID NO:2.7 or 5. In a further particular embodiment, an intracellular signaling domain comprises a TLR8 primary intracellular signaling domain comprising an amino acid sequence of SEQ ID NO:47 and a CD3ζ secondary intracellular signaling domain comprising an amino acid sequence of SEQ ID NO:27 or 5. In another particular embodiment, an intracellular signaling domain comprises a CD28 primary intracellular signaling domain comprising an amino acid sequence of SEQ ID NO:4 or 26 and a DAP12 secondary intracellular signaling domain comprising an amino acid sequence of SEQ ID NO:9. In some embodiments, an intracellular signaling domain comprises a combination of primary, secondary, and optionally tertiary intracellular signaling domain sequences as shown in Table 10.

Intracellular signaling domains may be derived from a mammalian species, including humans, primates, cows, horses, goats, sheep, dogs, cats, mice, rats, rabbits, guinea pigs, pigs, and transgenic species thereof.

The transmembrane domain of a chimeric Tim receptor connects and is positioned between the extracellular domain and the intracellular signaling domain. The transmembrane domain is a hydrophobic alpha helix that transverses the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). In one embodiment, the transmembrane domain is selected from the same molecule as the molecule from which the extracellular domain is derived. In another embodiment, the transmembrane domain is selected from the same molecule as the molecule from which the intracellular signaling domain is derived. For example, a chimeric Tim receptor may comprise a Tim4 binding domain and a Tim4 transmembrane domain. In another example, a chimeric Tim receptor may comprise a CD28 transmembrane domain and a CD28 costimulatory signaling domain. In certain embodiments, the transmembrane domain and the extracellular domain are derived from different molecules; the transmembrane domain and the intracellular signaling domain are derived from different molecules; or the transmembrane domain, extracellular domain, and intracellular signaling domain are all derived from different molecules. Examples of transmembrane domains that may be used in chimeric Tim receptors of the present disclosure include transmembrane domains from Tim1, Tim4, and CD28. An exemplary Tim1 transmembrane domain comprises or consists of an amino acid sequence of SEQ ID NO:8. An exemplary Tim4 transmembrane domain comprises or consists of an amino acid sequence of SEQ ID NO:6 or 23. An exemplary CD28 transmembrane domain comprises or consists of an amino acid sequence of SEQ ID NO:7. In certain embodiments, the transmembrane domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:6-8 or 23. In certain embodiments, the transmembrane domain comprises an amino acid sequence having at least about 1, 2, 3, 4, 5, 6, 7, 8, 9, or 10 amino acid modifications (e.g., deletion, additions, substitutions) to an amino acid sequence of any one of SEQ ID NOS:6-8 or 23.

Transmembrane domains may derived from any mammalian species, including humans, primates, cows, horses, goats, sheep, dogs, cats, mice, rats, rabbits, guinea pigs, pigs, and transgenic species thereof.

In certain embodiments, a chimeric Tim receptor comprises polynucleotide sequences derived from any mammalian species, including humans, primates, cows, horses, goats, sheep, dogs, cats, mice, rats, rabbits, guinea pigs, pigs, transgenic species thereof or any combination thereof. In certain embodiments, a chimeric Tim receptor is murine, chimeric, human, or humanized.

It is understood that direct fusion of one domain to another domain of a chimeric Tim receptor described herein does not preclude the presence of intervening junction amino acids. Junction amino acids may be natural or non-natural (e.g., resulting from the construct design of a chimeric protein). For example, junction amino acids may result from restriction enzyme sites used for joining one domain to another domain or cloning polynucleotides encoding chimeric Tim receptors into vectors.

### Exemplary Chimeric Tim Receptors

The component parts of a chimeric Tim receptor as disclosed herein can be selected and arranged in various combinations to provide a desired specificity and effector phenotype to a host cell.

Exemplary chimeric Tim receptors of the present disclosure are described in Table 4.

TABLE 4

| | | | Trans- | Primary | Secondary |
|---|---|---|---|---|---|
| Construct | IgV | Mucin | membrane | Signaling | Signaling |
| No. | Domain | Domain | Domain | Domain | Domain |
| 1 | Tim1 | Tim1 | Tim1 | Tim1 | CD3ζ |
| 2 | Tim1 | Tim1 | Tim1 | Tim4 | CD3ζ |
| 3 | Tim1 | Tim1 | CD28 | CD28 | |
| 4 | Tim1 | Tim1 | Tim1 | TRAF6 | |
| 5 | Tim1 | Tim1 | CD28 | TRAF6 | |
| 6 | Tim1 | Tim1 | Tim1 | TRAF2 | |
| 7 | Tim1 | Tim1 | CD28 | TRAF2 | |
| 8 | Tim1 | Tim1 | Tim1 | TLR8 | CD3ζ |
| 9 | Tim1 | Tim1 | CD28 | CD28 | DAP12 |
| 10 | Tim1 | Tim1 | Tim1 | CD28 | DAP12 |

Further exemplary chimeric Tim receptors are described in Table 5.

TABLE 5

| | | | Trans- | Primary | Secondary |
|---|---|---|---|---|---|
| Construct | IgV | Mucin | membrane | Signaling | Signaling |
| No. | Domain | Domain | Domain | Domain | Domain |
| 1′ | SEQ ID | SEQ ID | SEQ ID NO: 8 | SEQ ID | SEQ ID |
| | NO: 38 | NO: 39 | | NO: 44 | NO: 5 |
| 2′ | SEQ ID | SEQ ID | SEQ ID NO: 8 | SEQ ID | SEQ ID |
| | NO: 38 | NO: 39 | | NO: 45 | NO: 5 |
| 3′ | SEQ ID | SEQ ID | SEQ ID NO: 7 | SEQ ID | |
| | NO: 38 | NO: 39 | | NO: 4 | |
| 4′ | SEQ ID | SEQ ID | SEQ ID NO: 8 | SEQ ID | |
| | NO: 38 | NO: 39 | | NO: 46 | |
| 5′ | SEQ ID | SEQ ID | SEQ ID NO: 7 | SEQ ID | |
| | NO: 38 | NO: 39 | | NO: 46 | |
| 6′ | SEQ ID | SEQ ID | SEQ ID NO: 8 | SEQ ID | |
| | NO: 38 | NO: 39 | | NO: 48 | |
| 7′ | SEQ ID | SEQ ID | SEQ ID NO: 7 | SEQ ID | |
| | NO: 38 | NO: 39 | | NO: 48 | |
| 8′ | SEQ ID | SEQ ID | SEQ ID NO: 8 | SEQ ID | SEQ ID |
| | NO: 38 | NO: 39 | | NO: 47 | NO: 5 |
| 9′ | SEQ ID | SEQ ID | SEQ ID NO: 7 | SEQ ID | SEQ ID |
| | NO: 38 | NO: 39 | | NO: 4 | NO: 9 |
| 10′ | SEQ ID | SEQ ID | SEQ ID NO: 8 | SEQ ID | SEQ ID |
| | NO: 38 | NO: 39 | | NO: 4 | NO: 9 |

In some embodiments, a chimeric Tim receptor of the present disclosure comprises a construct of Table 4. In some embodiments, a chimeric Tim receptor of the present disclosure comprises a construct of Table 5.

In some embodiments, a chimeric Tim receptor of Construct 1 or Construct 1′ comprises amino acids 21-456 of SEQ ID NO:49. In some embodiments, a chimeric Tim receptor of Construct 1 or Construct 1′ comprises an amino acid sequence of SEQ ID NO:49.

In some embodiments, a chimeric Tim receptor of Construct 2 or Construct 2′ comprises amino acids 21-471 of SEQ ID NO:50. In some embodiments, a chimeric Tim receptor of Construct 2 or Construct 2′ comprises an amino acid sequence of SEQ ID NO:50.

In some embodiments, a chimeric Tim receptor of Construct 3 or Construct 3′ comprises amino acids 21-363 of SEQ ID NO:51. In some embodiments, a chimeric Tim receptor of Construct 3 or Construct 3′ comprises an amino acid sequence of SEQ ID NO:51.

In some embodiments, a chimeric Tim receptor of Construct 4 or Construct 4′ comprises amino acids 21-590 of SEQ ID NO:52. In some embodiments, a chimeric Tim receptor of Construct 4 or Construct 4′ comprises an amino acid sequence of SEQ ID NO:52.

In some embodiments, a chimeric Tim receptor of Construct 5 or Construct 5′ comprises amino acids 21-596 of SEQ ID NO:53. In some embodiments, a chimeric Tim receptor of Construct 5 or Construct 5′ comprises an amino acid sequence of SEQ LD NO:53.

In some embodiments, a chimeric Tim receptor of Construct 6 or Construct 6′ comprises amino acids 21-619 of SEQ ID NO:54. In some embodiments, a chimeric Tim receptor of Construct 6 or Construct 6′ comprises an amino acid sequence of SEQ ID NO:54.

In some embodiments, a chimeric Tim receptor of Construct 7 or Construct 7′ comprises amino acids 21-625 of SEQ ID NO:55. In some embodiments, a chimeric Tim receptor of Construct 7 or Construct 7′ comprises an amino acid sequence of SEQ ID NO:55.

In some embodiments, a chimeric Tim receptor of Construct 8 or Construct 8′ comprises amino acids 21-621 of SEQ ID NO:56. In some embodiments, a chimeric Tim receptor of Construct 8 or Construct 8′ comprises an amino acid sequence of SEQ LD NO:56.

In some embodiments, a chimeric Tim receptor of Construct 9 or Construct 9′ comprises amino acids 21-415 of SEQ ID NO:57. In some embodiments, a chimeric Tim receptor of Construct 9 or Construct 9′ comprises an amino acid sequence of SEQ ID NO:57.

In some embodiments, a chimeric Tim receptor of Construct 10 or Construct 10′ comprises amino acids 21-409 of SEQ ID NO:58& In some embodiments, a chimeric Tim receptor of Construct 10 or Construct 10′ comprises an amino acid sequence of SEQ ID NO:58.

Further exemplary chimeric Tim receptors of the present disclosure are described in Table 6.

TABLE 6

| | | | Trans- | Primary | Secondary |
|---|---|---|---|---|---|
| Construct | IgV | Mucin | membrane | Signaling | Signaling |
| No. | Domain | Domain | Domain | Domain | Domain |
| 11 | Tim4 | Tim4 | Tim4 | Tim4 | CD3ζ |
| 12 | Tim4 | Tim4 | Tim4 | Tim1 | CD3ζ |
| 13 | Tim4 | Tim4 | CD28 | CD28 | |
| 13A | Tim4 | Tim4 | CD28 | CD28 | CD3ζ |
| 14 | Tim4 | Tim4 | Tim4 | TRAF6 | |
| 15 | Tim4 | Tim4 | CD28 | TRAF6 | |
| 16 | Tim4 | Tim4 | Tim4 | TRAF2 | |
| 17 | Tim4 | Tim4 | CD28 | TRAF2 | |
| 18 | Tim4 | Tim4 | Tim1 | TLR8 | CD3ζ |

TABLE 7

| | | | | Primary | Secondary |
|---|---|---|---|---|---|
| Construct | Ig V | Mucin | Transmembrane | Signaling | Signaling |
| No. | Domain | Domain | Domain | Domain | Domain |
| 11′ | SEQ ID | SEQ ID | SEQ ID NO: 6 | SEQ ID | SEQ ID |
| | NO: 34 | NO: 35 | | NO: 45 | NO: 5 |
| 12′ | SEQ ID | SEQ ID | SEQ ID NO: 6 | SEQ ID | SEQ ID |
| | NO: 34 | NO: 35 | | NO: 44 | NO: 5 |
| 13′ | SEQ ID | SEQ ID | SEQ ID NO: 7 | SEQ ID | |
| | NO: 34 | NO: 35 | | NO: 4 | |
| 14′ | SEQ ID | SEQ ID | SEQ ID NO: 6 | SEQ ID | |
| | NO: 34 | NO: 35 | | NO: 46 | |
| 15′ | SEQ ID | SEQ ID | SEQ ID NO: 7 | SEQ ID | |
| | NO: 34 | NO: 35 | | NO: 46 | |
| 16′ | SEQ ID | SEQ ID | SEQ ID NO: 6 | SEQ ID | |
| | NO: 34 | NO: 35 | | NO: 48 | |
| 17′ | SEQ ID | SEQ ID | SEQ ID NO: 7 | SEQ ID | |
| | NO: 34 | NO: 35 | | NO: 48 | |
| 18′ | SEQ ID | SEQ ID | SEQ ID NO: 8 | SEQ ID | SEQ ID |
| | NO: 34 | NO: 35 | | NO: 47 | NO: 5 |

In some embodiments, a chimeric Tim receptor of the present disclosure comprises a construct of Table 6. In some embodiments, a chimeric Tim receptor of the present disclosure comprises a construct of Table 7.

In some embodiments, a chimeric Tim receptor of Construct 11 or Construct 11′ comprises amino acids 25-490 of SEQ ID NO:59. In some embodiments, a chimeric Tim receptor of Construct 11 or Construct 11′ comprises an amino acid sequence of SEQ ID NO:59.

In some embodiments, a chimeric Tim receptor of Construct 12 or Construct 12′ comprises amino acids 25-495 of SEQ ID NO:60. In some embodiments, a chimeric Tim receptor of Construct 12 or Construct 12′ comprises an amino acid sequence of SEQ ID NO:60.

In some embodiments, a chimeric Tim receptor of Construct 13 or Construct 13′ comprises amino acids 25-382 of SEQ ID NO:61. In some embodiments, a chimeric Tim receptor of Construct 13 or Construct 13′ comprises an amino acid sequence of SEQ ID NO:61.

In some embodiments, a chimeric Tim receptor of Construct 13A (TIM4 binding domain-CD28 TM—CD28 costim-CD3ζ amino acids 1-24 signal peptide)) comprises amino acids 25-383 of SEQ ID NO:71. In some embodiments, a chimeric Tim receptor of Construct 13A comprises an amino acid sequence of SEQ ID NO:71.

In some embodiments, a chimeric Tim receptor of Construct 14 or Construct 14′ comprises amino acids 25-609 of SEQ ID NO:62. In some embodiments, a chimeric Tim receptor of Construct 14 or Construct 14′ comprises an amino acid sequence of SEQ ID NO:62.

In some embodiments, a chimeric Tim receptor of Construct 15 or Construct 15′ comprises amino acids 25-615 of SEQ ID NO:63. In some embodiments, a chimeric Tim receptor of Construct 15 or Construct 15′ comprises an amino acid sequence of SEQ ID NO:63.

In some embodiments, a chimeric Tim receptor of Construct 16 or Construct 16′ comprises amino acids 25-638 of SEQ ID NO:64. In some embodiments, a chimeric Tim receptor of Construct 16 or Construct 16′ comprises an amino acid sequence of SEQ ID NO:64.

In some embodiments, a chimeric Tim receptor of Construct 17 or Construct 17′ comprises amino acids 25-644 of SEQ ID NO:65. In some embodiments, a chimeric Tim receptor of Construct 17 or Construct 17′ comprises an amino acid sequence of SEQ FD NO:65.

In some embodiments, a chimeric Tim receptor of Construct 18 or Construct 18′ comprises amino acids 25-640 of SEQ ID NO:66. In some embodiments, a chimeric Tim receptor of Construct 18 or Construct 18′ comprises an amino acid sequence of SEQ ID NO:66.

Further exemplary chimeric Tim receptors of the present disclosure are described in Table 8.

TABLE 8

| | | | | Primary | Secondary |
|---|---|---|---|---|---|
| Construct | IgV | Mucin | Transmembrane | Signaling | Signaling |
| No. | Domain | Domain | Domain | Domain | Domain |
| 19 | Tim4 | Tim1 | Tim1 | TLR8 | CD3ζ |
| 20 | Tim4 | Tim1 | Tim1 | CD28 | DAP12 |
| 21 | Tim4 | Tim1 | CD28 | CD28 | DAP12 |

TABLE 9

| | | | | Primary | Secondary |
|---|---|---|---|---|---|
| Construct | IgV | Mucin | Transmembrane | Signaling | Signaling |
| No. | Domain | Domain | Domain | Domain | Domain |
| 19 | SEQ ID | SEQ ID | SEQ ID NO: 8 | SEQ ID | SEQ ID |
| | NO: 34 | NO: 39 | | NO: 47 | NO: 5 |
| 20 | SEQ ID | SEQ ID | SEQ ID NO: 8 | SEQ ID | SEQ ID |
| | NO: 34 | NO: 39 | | NO: 4 | NO: 9 |
| 21 | SEQ ID | SEQ ID | SEQ ID NO: 7 | SEQ ID | SEQ ID |
| | NO: 34 | NO: 39 | | NO: 4 | NO: 9 |

In some embodiments, a chimeric Tim receptor of the present disclosure comprises a construct of Table 8. In some embodiments, a chimeric Tim receptor of the present disclosure comprises a construct of Table 9.

In some embodiments, a chimeric Tim receptor of Construct 19 or Construct 19′ comprises amino acids 25-628 of SEQ ID NO:67. In some embodiments, a chimeric Tim receptor of Construct 19 or Construct 19′ comprises an amino acid sequence of SEQ ID NO:67.

In some embodiments, a chimeric Tim receptor of Construct 20 or Construct 20′ comprises amino acids 25-416 of SEQ ID NO:68. In some embodiments, a chimeric Tim receptor of Construct 20 or Construct 20′ comprises an amino acid sequence of SEQ ID NO:68.

In some embodiments, a chimeric Tim receptor of Construct 21 or Construct 21 comprises amino acids 25-422 of SEQ ID NO:69. In some embodiments, a chimeric Tim receptor of Construct 21 or Construct 21′ comprises an amino acid sequence of SEQ ID NO:69.

Further exemplary chimeric Tim receptors are described in Table 10. In some embodiments, a chimeric Tim receptor of the present disclosure comprises a construct of Table 10. In some embodiments, a chimeric Tim receptor of the present disclosure does not include a chimeric Tim receptor having the amino acid sequence of SEQ ID NO:227, 238, 249, 72, or 73. In some embodiments, a chimeric Tim receptor of the present disclosure does not include a chimeric Tim receptor having the combination of components as described for constructs SEQ ID NO:227, 238, 249, 72, or 73.

In some embodiments, a chimeric Tim 4 receptor comprises the amino acid sequence of SEQ ID NO: 127 or the amino acid sequence of SEQ LD NO:127 absent the signal sequence (amino acids 1-24). In some embodiments, a chimeric Tim 4 receptor comprises the amino acid sequence of SEQ ID NO:128 or the amino acid sequence of SEQ ID NO:128 absent the signal sequence (amino acids 1-24). In some embodiments, a chimeric Tim 4 receptor comprises the amino acid sequence of SEQ ID NO:129 or the amino acid sequence of SEQ ID NO:129 absent the signal sequence (amino acids 1-24). In some embodiments, a chimeric Tim 4 receptor comprises the amino acid sequence of SEQ ID NO:130 or the amino acid sequence of SEQ ID NO:130 absent the signal sequence (amino acids 1-24). In some embodiments, a chimeric Tim 4 receptor comprises the amino acid sequence of SEQ ID NO: 131 or the amino acid sequence of SEQ ID NO: 131 absent the signal sequence (amino acids 1-24). In some embodiments, a chimeric Tim 4 receptor comprises the amino acid sequence of SEQ ID NO: 132 or the amino acid sequence of SEQ ID NO:132 absent the signal sequence (amino acids 1-24). In some embodiments, a chimeric Tim 4 receptor comprises the amino acid sequence of SEQ ID NO: 133 or the amino acid sequence of SEQ ID NO:133 absent the signal sequence (amino acids 1-24). In some embodiments, a chimeric Tim 4 receptor comprises the amino acid sequence of SEQ ID NO: 134 or the amino acid sequence of SEQ ID NO:134 absent the signal sequence (amino acids 1-24). In some embodiments, a chimeric Tim 4 receptor comprises the amino acid sequence of SEQ ID NO:135 or the amino acid sequence of SEQ ID NO: 135 absent the signal sequence (amino acids 1-24). In some embodiments, a chimeric Tim 4 receptor comprises the amino acid sequence of SEQ ID NO:136 or the amino acid sequence of SEQ ID NO: 136 absent the signal sequence (amino acids 1-24). In some embodiments, a chimeric Tim 4 receptor comprises the amino acid sequence of SEQ ID NO:137 or the amino acid sequence of SEQ ID NO: 137 absent the signal sequence (amino acids 1-24). In some embodiments, a chimeric Tim 4 receptor comprises the amino acid sequence of SEQ ID NO: 138 or the amino acid sequence of SEQ ID NO:138 absent the signal sequence (amino acids 1-24). In some embodiments, a chimeric Tim 4 receptor comprises the amino acid sequence of SEQ ID NO:139 or the amino acid sequence of SEQ ID NO:139 absent the signal sequence (amino acids 1-24). In some embodiments, a chimeric Tim 4 receptor comprises the amino acid sequence of SEQ ID NO:140 or the amino acid sequence of SEQ ID NO:140 absent the signal sequence (amino acids 1-24). In some embodiments, a chimeric Tim 4 receptor comprises the amino acid sequence of SEQ ID NO:141 or the amino acid sequence of SEQ ID NO:141 absent the signal sequence (amino acids 1-24). In some embodiments, a chimeric Tim 4 receptor comprises the amino acid sequence of SEQ ID NO:142 or the amino acid sequence of SEQ ID NO:142 absent the signal sequence (amino acids 1-24). In some embodiments, a chimeric Tim 4 receptor comprises the amino acid sequence of SEQ ID NO: 143 or the amino acid sequence of SEQ 1D NO:143 absent the signal sequence (amino acids 1-24). In some embodiments, a chimeric Tim 4 receptor comprises the amino acid sequence of SEQ ID NO:144 or the amino acid sequence of SEQ ID NO:144 absent the signal sequence (amino acids 1-24). In some embodiments, a chimeric Tim 4 receptor comprises the amino acid sequence of SEQ ID NO:145 or the amino acid sequence of SEQ ID NO: 145 absent the signal sequence (amino acids 1-24). In some embodiments, a chimeric Tim 4 receptor comprises the amino acid sequence of SEQ ID NO: 146 or the amino acid sequence of SEQ ID NO: 146 absent the signal sequence (amino acids 1-24). In some embodiments, a chimeric Tim 4 receptor comprises the amino acid sequence of SEQ ID NO: 147 or the amino acid sequence of SEQ ID NO: 147 absent the signal sequence (amino acids 1-24). In some embodiments, a chimeric Tim 4 receptor comprises the amino acid sequence of SEQ ID NO: 148 or the amino acid sequence of SEQ ID NO:148 absent the signal sequence (amino acids 1-24). In some embodiments, a chimeric Tim 4 receptor comprises the amino acid sequence of SEQ ID NO: 149 or the amino acid sequence of SEQ ID NO:149 absent the signal sequence (amino acids 1-24). In some embodiments, a chimeric Tim 4 receptor comprises the amino acid sequence of SEQ ID NO:150 or the amino acid sequence of SEQ ID NO: 150 absent the signal sequence (amino acids 1-24). In some embodiments, a chimeric Tim 4 receptor comprises the amino acid sequence of SEQ ID NO:151 or the amino acid sequence of SEQ ID NO:151 absent the signal sequence (amino acids 1-24). In some embodiments, a chimeric Tim 4 receptor comprises the amino acid sequence of SEQ ID NO:152 or the amino acid sequence of SEQ ID NO: 152 absent the signal sequence (amino acids 1-24). In some embodiments, a chimeric Tim 4 receptor comprises the amino acid sequence of SEQ ID NO:153 or the amino acid sequence of SEQ ID NO:153 absent the signal sequence (amino acids 1-24). In some embodiments, a chimeric Tim 4 receptor comprises the amino acid sequence of SEQ ID NO: 154 or the amino acid sequence of SEQ ID NO:154 absent the signal sequence (amino acids 1-24). In some embodiments, a chimeric Tim 4 receptor comprises the amino acid sequence of SEQ ID NO:155 or the amino acid sequence of SEQ ID NO:155 absent the signal sequence (amino acids 1-24).

TABLE 10

| Construct | | Signal | Extracellular | Transmembrane | Intracellular | Intracellular | Intracellular |
|---|---|---|---|---|---|---|---|
| Number | Construct Description | Peptide | Domain | Domain | Domain #1 | Domain #2 | Domain #3 |
| pCTX1161 | hTIM4-TIM4-CD28tm- | hTIM4 | TIM4 | CD28 | CD28 | CD3z | |
| [SEQ ID | CD28icd-CD3zICD | [SEQ ID | [SEQ ID | [SEQ ID | [SEQ ID NO: 4] | [SEQ ID NO: 27] | |
| NO: 227] | | NO: 218] | NO: 219] | NO: 220] | | | |
| pCTX1162 | hTIM4-TIM4-CD28tm- | hTIM4 | TIM4 | CD28 | CD28 | TLR2 | CD3z |
| [SEQ ID | CD28icd-TLR2ICD- | [SEQ ID | [SEQ ID | [SEQ ID | [SEQ ID NO: 4] | [SEQ ID | [SEQ ID NO: 27] |
| NO: 228] | CD3zICD | NO: 218] | NO: 219] | NO: 220] | | NO: 222] | |
| pCTX1163 | hTIM4-TIM4-CD28tm- | hTIM4 | TIM4 | CD28 | CD28 | CD3z | TLR2 |
| [SEQ ID | CD28icd-CD3zICD- | [SEQ ID | [SEQ ID | [SEQ ID | [SEQ ID NO: 4] | [SEQ ID NO: 27] | [SEQ ID |
| NO: 229] | TLR2ICD | NO: 218] | NO: 219] | NO: 220] | | | NO: 222] |
| pCTX1164 | hTIM4-TIM4-CD28tm- | hTIM4 | TIM4 | CD28 | CD28 | TLR8 | CD3z |
| [SEQ ID | CD28icd-TLR8ICD- | [SEQ ID | [SEQ ID | [SEQ ID | [SEQ ID NO: 4] | [SEQ ID NO: 47] | [SEQ ID NO: 27] |
| NO: 230] | CD3zICD | NO: 218] | NO: 219] | NO: 220] | | | |
| pCTX1165 | hTIM4-TIM4-CD28tm- | hTIM4 | TIM4 | CD28 | CD28 | CD3z | TLR8 |
| [SEQ ID | CD28icd-CD3zICD- | [SEQ ID | [SEQ ID | [SEQ ID | [SEQ ID NO: 4] | [SEQ ID NO: 27] | [SEQ ID NO: 47] |
| NO: 231] | TLR8ICD | NO: 218] | NO: 219] | NO: 220] | | | |
| pCTX1166 | hTIM4-TIM4-CD28tm- | hTIM4 | TIM4 | CD28 | TLR2 | CD3z | |
| [SEQ ID | TLR2ICD-CD3zICD | [SEQ ID | [SEQ ID | [SEQ ID | [SEQ ID | [SEQ ID NO: 27] | |
| NO: 232] | | NO: 218] | NO: 219] | NO: 220] | NO: 222] | | |
| pCTX1167 | hTIM4-TIM4-CD28tm- | hTIM4 | TIM4 | CD28 | CD3z | TLR2 | |
| [SEQ ID | CD3zICD-TLR2ICD | [SEQ ID | [SEQ ID | [SEQ ID | [SEQ ID NO: 27] | [SEQ ID | |
| NO: 233] | | NO: 218] | NO: 219] | NO: 220] | | NO: 222] | |
| pCTX1168 | hTIM4-TIM4-CD28tm- | hTIM4 | TIM4 | CD28 | TLR8 | CD3z | |
| [SEQ ID | TLR8ICD-CD3zICD | [SEQ ID | [SEQ ID | [SEQ ID | [SEQ ID NO: 47] | [SEQ ID NO: 27] | |
| NO: 234] | | NO: 218] | NO: 219] | NO: 220] | | | |
| pCTX1169 | hTIM4-TIM4-CD28tm- | bTIM4 | TIM4 | CD28 | CD3z | TLR8 | |
| [SEQ ID | CD3zICD-TLR8ICD | [SEQ ID | [SEQ ID | [SEQ ID | [SEQ ID NO: 27] | [SEQ ID NO: 47] | |
| NO: 235] | | NO: 218] | NO: 219] | NO: 220] | | | |
| pCTX1170 | hTIM4-TIM4-CD28tm- | hTIM4 | TIM4 | CD28 | TRAF6 | CD3z | |
| [SEQ ID | TRAF6ICD-CD3zICD | [SEQ ID | [SEQ ID | [SEQ ID | [SEQ ID NO: 46] | [SEQ ID NO: 27] | |
| NO: 236] | | NO: 218] | NO: 219] | NO: 220] | | | |
| pCTX1171 | hTIM4-TIM4-CD28tm- | hTIM4 | TIM4 | CD28 | CD3z | TRAF6 | |
| [SEQ ID | CD3zICD-TRAF6ICD | [SEQ ID | [SEQ ID | [SEQ ID | [SEQ ID NO: 27] | [SEQ ID NO: 46] | |
| NO: 237] | | NO: 218] | NO: 219] | NO: 220] | | | |
| pCTX1172 | hTIM4-TIM4-TIM4tm- | hTIM4 | TIM4 | TIM4 | CD28 | CD3z | |
| [SEQ ID | CD28icd-CD3zICD | [SEQ ID | [SEQ ID | [SEQ ID | [SEQ ID NO: 4] | [SEQ ID NO: 27] | |
| NO: 238] | | NO: 218] | NO: 219] | NO: 221] | | | |
| pCTX1173 | hTIM4-TIM4-TIM4tm- | hTIM4 | TIM4 | TIM4 | CD28 | TLR2 | CD3z |
| [SEQ ID | CD28icd-TLR2ICD- | [SEQ ID | [SEQ ID | [SEQ ID | [SEQ ID NO: 4] | [SEQ ID | [SEQ ID NO: 27] |
| NO: 239] | CD3zICD | NO: 218] | NO: 219] | NO: 221] | | NO: 222] | |
| pCTX1174 | hTIM4-TIM4-TIM4tm- | hTIM4 | TIM4 | TIM4 | CD28 | CD3z | TLR2 |
| [SEQ ID | CD28icd-CD3zICD- | [SEQ ID | [SEQ ID | [SEQ ID | [SEQ ID NO: 4] | [SEQ ID NO: 27] | [SEQ ID |
| NO: 240] | TLR2ICD | NO: 218] | NO: 219] | NO: 221] | | | NO: 222] |
| pCTX1175 | hTIM4-TIM4-TIM4tm- | hTIM4 | TIM4 | TIM4 | CD28 | TLR8 | CD3z |
| [SEQ ID | CD28icd-TLR8ICD- | [SEQ ID | [SEQ ID | [SEQ ID | [SEQ ID NO: 4] | [SEQ ID NO: 47] | [SEQ ID NO: 27] |
| NO: 241] | CD3zICD | NO: 218] | NO: 219] | NO: 221] | | | |
| pCTX1176 | hTIM4-TIM4-TIM4tm- | hTIM4 | TIM4 | TIM4 | CD28 | CD3z | TLR8 |
| [SEQ ID | CD28icd-CD3zICD- | [SEQ ID | [SEQ ID | [SEQ ID | [SEQ ID NO: 4] | [SEQ ID NO: 27] | [SEQ ID NO: 47] |
| NO: 242] | TLR8ICD | NO: 218] | NO: 219] | NO: 221] | | | |
| pCTX1177 | hTIM4-TIM4-TIM4tm- | hTIM4 | TIM4 | TIM4 | TLR2 | CD3z | |
| [SEQ ID | TLR2ICD-CD3zICD | [SEQ ID | [SEQ ID | [SEQ ID | [SEQ ID | [SEQ ID NO: 27] | |
| NO: 243] | | NO: 218] | NO: 219] | NO: 221] | NO: 222] | | |
| pCTX1178 | hTIM4-TIM4-TIM4tm- | hTIM4 | TIM4 | TIM4 | CD3z | TLR2 | |
| [SEQ ID | CD3zICD-TLR2ICD | [SEQ ID | [SEQ ID | [SEQ ID | [SEQ ID NO: 27] | [SEQ ID | |
| NO: 244] | | NO: 218] | NO: 2191] | NO: 221] | | NO: 222] | |
| pCTX1179 | hTIM4-TIM4-TIM4tm- | hTIM4 | TIM4 | TIM4 | TLR8 | CD3z | |
| [SEQ ID | TLR8ICD-CD3zICD | [SEQ ID | [SEQ ID | [SEQ ID | [SEQ ID NO: 47] | [SEQ ID NO: 27] | |
| NO: 245] | | NO: 218] | NO: 219] | NO: 221] | | | |
| pCTX1180 | hTIM4-TIM4-TIM4tm- | hTIM4 | TIM4 | TIM4 | CD3z | TLR8 | |
| [SEQ ID | CD3zICD-TLR8ICD | [SEQ ID | [SEQ ID | [SEQ ID | [SEQ ID NO: 27] | [SEQ ID NO: 47] | |
| NO: 246] | | NO: 218] | NO: 219] | NO: 221] | | | |
| pCTX1181 | hTIM4-TIM4-TIM4tm- | hTIM4 | TIM4 | TIM4 | TRAF6 | CD3z | |
| [SEQ ID | TRAF6ICD-CD3zICD | [SEQ ID | [SEQ ID | [SEQ ID | [SEQ ID NO: 46] | [SEQ ID NO: 27] | |
| NO: 247] | | NO: 218] | NO: 219] | NO: 221] | | | |
| pCTX1182 | hTIM4-TIM4-TIM4tm- | hTIM4 | TIM4 | TIM4 | CD3z | TRAF6 | |
| [SEQ ID | CD3zICD-TRAF6ICD | [SEQ ID | [SEQ ID | [SEQ ID | [SEQ ID NO: 27] | [SEQ ID NO: 46] | |
| NO: 248] | | NO: 218] | NO: 219] | NO: 221] | | | |
| pCTX1183 | hTIM4-TIM4-TIM4tm- | hTIM4 | TIM4 | TIM4 | TIM4 | | |
| [SEQ ID | TIM4ICD | [SEQ ID | [SEQ ID | [SEQ ID | [SEQ ID | | |
| NO: 249] | | NO: 218] | NO: 219] | NO: 221] | NO: 225] | | |
| pCTX1184 | hTIM4-TIM4-TIM4tm- | hTIM4 | TIM4 | TIM4 | TIM4 | TLR2 | CD3z |
| [SEQ ID | TIM4ICD-TLR2-CD3z | [SEQ ID | [SEQ ID | [SEQ ID | [SEQ ID | [SEQ ID NO: 87] | [SEQ ID NO: 27] |
| NO: 250] | | NO: 218] | NO: 219] | NO: 221] | NO: 224] | | |
| pCTX1185 | hTIM4-TIM4-TIM4tm- | hTIM4 | TIM4 | TIM4 | TIM4 | CD3z | TLR2 |
| [SEQ ID | TIM4ICD-CD3z-TLR2 | [SEQ ID | [SEQ ID | [SEQ ID | [SEQ ID | [SEQ ID NO: 27] | [SEQ ID |
| NO: 251] | | NO: 218] | NO: 219] | NO: 221] | NO: 224] | | NO: 222] |
| pCTX1186 | hTIM4-TIM4-TIM4tm- | hTIM4 | TIM4 | TIM4 | TIM4 | TLR8 | CD3z |
| [SEQ ID | TIM4ICD-TLR8-CD3z | [SEQ ID | [SEQ ID | [SEQ ID | [SEQ ID | [SEQ ID NO: 47] | [SEQ ID NO: 27] |
| NO: 252] | | NO: 218] | NO: 219] | NO: 221] | NO: 224] | | |
| pCTX1187 | hTIM4-TIM4-TIM4tm- | hTIM4 | TIM4 | TIM4 | TIM4 | CD3z | TLR8 |
| [SEQ ID | TIM4ICD-CD3z-TLR8 | [SEQ ID | [SEQ ID | [SEQ ID | [SEQ ID | [SEQ ID NO: 27] | [SEQ ID NO: 47] |
| NO: 253] | | NO: 218] | NO: 219] | NO: 221] | NO: 224] | | |
| pCTX1189 | hTIM4-TIM4-TIM4tm- | hTIM4 | TIM4 | TIM4 | TLR2 | | |
| [SEQ ID | TLR2ICD | [SEQ ID | [SEQ ID | [SEQ ID | [SEQ ID | | |
| NO: 72] | | NO: 218] | NO: 219] | NO: 221] | NO: 222] | | |
| pCTX1190 | hTIM4-TIM4-TIM4tm- | hTIM4 | TIM4 | TIM4 | TLR8 | | |
| [SEQ ID | TLR8ICD | [SEQ ID | [SEQ ID | [SEQ ID | [SEQ ID NO: 47] | | |
| NO: 73] | | NO: 218] | NO: 219] | NO: 221] | | | |

### Polynucleotides, Vectors and Host Cells

In certain aspects, the present disclosure provides nucleic acid molecules that encode any one or more of the chimeric Tim receptors described herein. A nucleic acid may refer to a single- or double-stranded DNA, cDNA, or RNA, and may include a positive and a negative strand of the nucleic acid which complement one another, including antisense DNA, cDNA, and RNA. A nucleic acid may be naturally occurring or synthetic forms of DNA or RNA. The nucleic acid sequences encoding a desired chimeric Tim receptor can be obtained or produced using recombinant methods known in the art using standard techniques, such as by screening libraries from cells expressing the desired sequence or a portion thereof, by deriving the sequence from a vector known to include the same, or by isolating the sequence or a portion thereof directly from cells or tissues containing the same as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). Alternatively, the sequence of interest can be produced synthetically, rather than being cloned.

Polynucleotides encoding the chimeric Tim receptor compositions provided herein may be derived from any animal, such as humans, primates, cows, horses, sheep, dogs, cats, mice, rats, rabbits, guinea pigs, pigs, or a combination thereof. In certain embodiments, a polynucleotide encoding the chimeric Tim receptor is from the same animal species as the host cell into which the polynucleotide is inserted.

The polynucleotides encoding chimeric Tim receptors of the present disclosure may be operatively linked to expression control sequences. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion.

In certain embodiments, a polynucleotide encoding a chimeric Tim receptor comprises a sequence encoding a signal peptide (also referred to as leader peptide or signal sequence) at the 5′-end for targeting of the precursor protein to the secretory pathway. The signal peptide is optionally cleaved from the N-terminus of the extracellular domain during cellular processing and localization of the chimeric Tim receptor to the host cell membrane. A polypeptide from which a signal peptide sequence has been cleaved or removed may also be called a mature polypeptide. Examples of signal peptides that may be used in the chimeric Tim receptors of the present disclosure include signal peptides derived from endogenous secreted proteins, including, e.g., GM-CSF (amino acid sequence of SEQ ID NO:10), Tim1 (amino acid sequence of SEQ ID NO:40), or Tim4 (amino acid sequence of SEQ ID NO:11 or 25). In certain embodiments, a polynucleotide sequence encodes a mature chimeric Tim receptor polypeptide, or a polypeptide sequence comprises a mature chimeric Tim receptor polypeptide. It is understood by persons of skill in the art that for sequences disclosed herein that include a signal peptide sequence, the signal peptide sequence may be replaced with another signal peptide that is capable of trafficking the encoded protein to the extracellular membrane.

In certain embodiments, a chimeric Tim receptor encoding polynucleotide of the present disclosure is codon optimized for efficient expression in a target host cell comprising the polynucleotide (see, e.g. Scholten et al., Clin. Immunol. 119:135-145 (2006)). As used herein, a “codon optimized” polynucleotide comprises a heterologous polynucleotide having codons modified with silent mutations corresponding to the abundances of tRNA in a host cell of interest.

A single polynucleotide molecule may encode one, two, or more chimeric Tim receptors according to any of the embodiments disclosed herein. A polynucleotide encoding more than one transgene may comprise a sequence (e.g., IRES, viral 2A peptide) disposed between each gene for multicistronic expression.

Polynucleotides encoding at least two transgenes (e.g., a chimeric Tim receptor and CAR) provided in the present disclosure may be used to compose tandem expression cassettes. A tandem expression cassette refers to a component of a vector nucleic acid comprising at least two transgenes under the control of, or operatively linked to, the same set of regulatory sequences for tandem or co-expression of the at least two transgenes. Regulatory sequences that may be used in tandem expression cassettes of the present disclosure include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; sequences that enhance protein secretion, or any combination thereof.

In one aspect, the present disclosure provides a tandem expression cassette comprising a polynucleotide encoding a chimeric Tim receptor of the present disclosure and a polynucleotide encoding a cellular immunotherapy agent (e.g., a CAR, TCR, etc.).

In certain embodiments, a tandem expression cassette can be constructed to optimize spatial and temporal control. For example, a tandem expression cassette can include promoter elements to optimize spatial and temporal control. In some embodiments, a tandem expression cassette includes tissue specific promoters or enhancers that enable specific induction of a tandem expression cassette to an organ, a cell type (e.g., immune cell), or a pathologic microenvironment, such as a tumor or infected tissue. An “enhancer” is an additional promoter element that can function either cooperatively or independently to activate transcription. In certain embodiments, a tandem expression cassette includes a constitutive promoter. An exemplary constitutive promoter for use in tandem expression cassettes of the present disclosure is an EF-1α promoter. In certain embodiments, a tandem expression cassette includes an inducible promoter. In certain embodiments, a tandem expression cassette includes a tissue specific promoter.

The at least two transgenes contained within the tandem expression cassettes may be in any order. For example, a tandem expression cassette comprising a polynucleotide encoding a chimeric Tim receptor and a polynucleotide encoding a CAR may be arranged from 5′ to 3′: chimeric Tim receptor-CAR, or CAR-chimeric Tim receptor.

In certain embodiments, receptors that comprise two or more polypeptide chains that associate to form a multimer or complex may be encoded by two or more polynucleotide molecules within a tandem expression construct. Exemplary multimeric receptors contemplated for expression in tandem expression constructs of the present disclosure include multichain CARs, TCRs, TCR-CARs, and TRuC™ constructs. Accordingly, exemplary tandem expression cassette embodiments encoding a chimeric Tim receptor and a ICR may comprise a polynucleotide encoding a chimeric Tim receptor, a polynucleotide encoding a TCRα chain polypeptide, and a polynucleotide encoding a TCRβ chain polypeptide.

In certain embodiments, tandem expression cassettes of the present disclosure may comprise an internal ribosome entry site (IRES) or peptide cleavage site such as a furin cleavage site or viral 2A peptide, disposed between each polynucleotide contained within the tandem expression cassette to allow for co-expression of multiple proteins from a single mRNA. For example, an IRES, furin cleavage site, or viral 2A peptide may be disposed between a polynucleotide encoding a chimeric Tim receptor and a polynucleotide encoding a CAR within a tandem expression cassette. In another example, an IRES, furin cleavage site, or viral 2A peptide may be disposed between earth of a polynucleotide encoding a chimeric Tim receptor, a polynucleotide encoding a TCRα chain polypeptide, and a polynucleotide encoding a TCRβ chain polypeptide. In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of any one of SEQ ID NOs:12, 28, 29, or 30. An exemplary P2A peptide comprises an amino acid sequence of SEQ ID NO: 13 or 31. An exemplary E2A peptide sequence comprises an amino acid sequence of SEQ ID NO: 14. An exemplary F2A peptide sequence comprises an amino acid sequence of SEQ LD NO:15.

Certain embodiments of tandem expression cassettes of the present disclosure comprise a polynucleotide encoding a CAR/or TCR specific for a target antigen (e.g., tumor antigen) and a polynucleotide encoding a chimeric Tim receptor of the present disclosure. Upon binding a target cell expressing the target antigen by the CAR/or TCR, a cell modified to express such a tandem expression cassette induces apoptosis of the target cell. Apoptosis induces exposure of pro-engulfment markers on the target cell, such as phosphatidylserine, which may then target the damaged or apoptotic cells for engulfment by the chimeric Tim receptor.

A polynucleotide encoding a desired chimeric Tim receptor can be inserted into an appropriate vector, e.g., a viral vector, non-viral plasmid vector, and non-viral vectors, such as lipid-based DNA vectors, modified mRNA (modRNA), self-amplifying mRNA, CELiD, and transposon-mediated gene transfer (PiggyBac, Sleeping Beauty), for introduction into a host cell of interest (e.g., an immune cell). Polynucleotides encoding a chimeric Tim receptor of the present disclosure can be cloned into any suitable vector, such as an expression vector, a replication vector, a probe generation vector, or a sequencing vector. In certain embodiments, a polynucleotide encoding the extracellular domain, a polynucleotide encoding the transmembrane domain, and a polynucleotide encoding the intracellular signaling domain are joined together into a single polynucleotide and then inserted into a vector. In other embodiments, a polynucleotide encoding the extracellular domain, a polynucleotide encoding the transmembrane domain, and a polynucleotide encoding the intracellular signaling domain may be inserted separately into a vector such that the expressed amino acid sequence produces a functional chimeric Tim receptor. A vector that encodes a chimeric Tim receptor is referred to herein as a “chimeric Tim receptor vector.”

In certain embodiments, a vector comprises a polynucleotide encoding one chimeric Tim receptor. In certain embodiments, a vector comprises one polynucleotide encoding two or more chimeric Tim receptors. In certain embodiments, a single polynucleotide encoding two or more chimeric Tim receptors is cloned into a cloning site and expressed from a single promoter, with each chimeric Tim receptor sequence separated from each other by an internal ribosomal entry site (IES), furin cleavage site, or viral 2A peptide to allow for co-expression of multiple genes from a single open reading frame (e.g., a multicistronic vector). In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of SEQ ID NO: 12, 28, 29, or 30. An exemplary P2A peptide comprises an amino acid sequence of SEQ ID NO:13 or 31. An exemplary E2A peptide sequence comprises an amino acid sequence of SEQ ID NO:14. An exemplary F2A peptide sequence comprises an amino acid sequence of SEQ ID NO:15.

In certain embodiments, a vector comprises two or more polynucleotides, each polynucleotide encoding a chimeric Tim receptor. The two or more polynucleotides encoding chimeric Tim receptors may be cloned sequentially into a vector at different cloning sites, with each chimeric Tim receptor expressed under the regulation of different promoters. In certain embodiments, vectors that allow long-term integration of a transgene and propagation to daughter cells are utilized. Examples include viral vectors such as, adenovirus, adeno-associated virus, vaccinia virus, herpes viruses, cytomegalovirus, pox virus, or retroviral vectors, such as lentiviral vectors. Vectors derived from lentivirus can be used to achieve long-term gene transfer and have added advantages over vectors including the ability to transduce non-proliferating cells, such as hepatocytes, and low immunogenicity.

A vector that encodes a core virus is referred to herein as a “viral vector.” There are a large number of available viral vectors suitable for use with the compositions of the instant disclosure, including those identified for human gene therapy applications (see Pfeifer and Verme, Ann. Rev. Genomics Hum. Genet. 2:177, 2001). Suitable viral vectors include vectors based on RNA viruses, such as retrovirus-derived vectors, e.g., Maloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing chimeric receptor transgenes are known in the art and have been previous described, for example, in U.S. Pat. No. 8,119,772; Walchli et al., PLoS One 6:327930, 2011; Zhao et al., J. Immunol. 174:415, 2005; Engels et al., Hum. Gene Ther. 14:1155, 2003; Frecha et al., Mol. Ther. 18:1748, 2010; Verhoeyen et al., Methods Mol. Biol. 506:97, 2009. Retroviral and lentiviral vector constructs and expression systems are also commercially available.

In certain embodiments, a viral vector is used to introduce a non-endogenous polynucleotide encoding a chimeric Tim receptor to a host cell. A viral vector may be a retroviral vector or a lentiviral vector. A viral vector may also include a nucleic acid sequence encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising a fluorescent protein (e.g., green, yellow), an extracellular domain of human CD2, or a truncated human EGFR (EGFRt or tEGFR; see Wang et al., Blood 118:1255, 2011). An exemplary tEGFR comprises an amino acid sequence of SEQ ID NO: 16. When a viral vector genome comprises a plurality of genes to be expressed in a host cell as separate proteins from a single transcript, the viral vector may also comprise additional sequences between the two (or more) genes allowing for multicistronic expression. Examples of such sequences used in viral vectors include internal ribosome entry sites (IRES), furin cleavage sites, viral 2A peptides (e.g., T2A, P2A, E2A, F2A), or any combination thereof.

Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky e al., Gen Ther. 5: 1517, 1998).

Other viral vectors recently developed for gene therapy uses can also be used with the compositions and methods of this disclosure. Such vectors include those derived from baculoviruses and r-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

In certain embodiments, a chimeric Tim receptor vector can be constructed to optimize spatial and temporal control. For example, a chimeric Tim receptor vector can include promoter elements to optimize spatial and temporal control. In some embodiments, a chimeric Tim receptor vector includes tissue specific promoters or enhancers that enable specific induction of a chimeric Tim receptor to an organ, a cell type (e.g., immune cell), or a pathologic microenvironment, such as a tumor or infected tissue. An “enhancer” is an additional promoter element that can function either cooperatively or independently to activate transcription. In certain embodiments, a chimeric Tim receptor vector includes a constitutive promoter. In certain embodiments, a chimeric Tim receptor vector includes an inducible promoter. In certain embodiments, a chimeric Tim receptor vector includes a tissue specific promoter.

In certain embodiments, a chimeric Tim receptor vector can include a gene encoding a homing receptor, such as CCR4 or CXCR4, to improve homing and antitumor activity in vivo.

Where temporal control is desired, a chimeric Tim receptor vector may include an element that allows for inducible depletion of transduced cells. For example, such a vector may include an inducible suicide gene. A suicide gene may be an apoptotic gene or a gene that confers sensitivity to an agent (e.g., a drug). Exemplary suicide genes include chemically inducible caspase 9 (iCASP9) (U.S. Patent Publication No. 2013/0071414), chemically inducible Fas, or Herpes simplex virus thymidine kinase (HSV-TK), which confers sensitivity to ganciclovir. In further embodiments, a chimeric Tim receptor vector can be designed to express a known cell surface antigen that, upon infusion of an associated antibody, enables depletion of transduced cells. Examples of cell surface antigens and their associated antibodies that may be used for depletion of transduced cells include CD20 and Rituximab, RQR8 (combined CD34 and CD20 epitopes, allowing CD34 selection and anti-CD20 deletion) and Rituximab, and EGFR and Cetuximab.

Inducible vector systems, such as the tetracycline (Tet)-On vector system which activates transgene expression with doxycycline (Heinz et al., Hum. Gene Ther. 2011, 22:166-76) may also be used for inducible chimeric Tim receptor expression. Inducible chimeric Tim receptor expression may be also accomplished via retention using a selective hook (RUSH) system based on streptavidin anchored to the membrane of the endoplasmic reticulum through a hook and a streptavidin binding protein introduced into the chimeric Tim receptor structure, where addition of biotin to the system leads to the release of the chimeric Tim receptor from the endoplasmic reticulum (Agaugue et al., 2015, Mol. Ther. 23(Suppl. 1):S88).

In certain embodiments, a cell, such as an immune cell, obtained from a subject may be engineered into a non-natural or recombinant cell (e.g., a non-natural or recombinant immune cell) by introducing a polynucleotide that encodes a chimeric Tim receptor as described herein, whereby the cell expresses a cell surface localized chimeric Tim receptor. In certain embodiments, a host cell is an immune cell, such as a myeloid progenitor cell or a lymphoid progenitor cell. Exemplary immune cells that may be modified to comprise a polynucleotide encoding a chimeric Tim receptor or a vector comprising a polynucleotide encoding a chimeric Tim receptor include a T cell, a natural killer cell, a B cell, a lymphoid precursor cell, an antigen presenting cell, a dendritic cell, a Langerhans cell, a myeloid precursor cell, a mature myeloid cell, a monocyte, or a macrophage.

In certain embodiments, a B cell is genetically modified to express one or more chimeric Tim receptors. B cells possess certain properties that may be advantageous as host cells, including: trafficking to sites of inflammation, capable of internalizing and presenting antigen, capable of costimulating T cells, highly proliferative, and self-renewing (persist for life). In certain embodiments, a chimeric Tim receptor modified B cell is capable of digesting an engulfed target cell or engulfed target particle into smaller peptides and presenting them to T cells via an MHC molecule. Antigen presentation by a chimeric Tim receptor modified B cell may contribute to antigen spreading of the immune response to non-targeted antigens. B cells include progenitor or precursor cells committed to the B cell lineage (e.g., pre-pro-B cells, pro-B cells, and pre-B cells); immature and inactivated B cells; or mature and functional or activated B cells. In certain embodiments, B cells may be naïve B cells, plasma cells, regulatory B cells, marginal zone B cells, follicular B cells, lymphoplasmacytoid cell, plasmablast cell, memory B cells, or any combination thereof. Memory B cells may be distinguished from naïve B cells by expression of CD27, which is absent on naïve B cells. In certain embodiments, the B cells can be primary cells or cell lines derived from human, mouse, rat, or other mammals. B cell lines are well known in the art. If obtained from a mammal, a B cell can be obtained from numerous sources, including blood, bone marrow, spleen, lymph node, or other tissues or fluids. A B cell composition may be enriched or purified.

In certain embodiments, a T cell is genetically modified to express one or more chimeric Tim receptors. Exemplary T cells include CD4⁺ helper, CD8⁺ effector (cytotoxic), naïve (CD45 RA+, CCR7+, CD62L+, CD27+, CD45RO−), central memory (CD45RO⁺, CD62L⁺, CD8⁺), effector memory (CD45RA⁺, CD45RO−, CCR7−, CD62L−, CD27−), T memory stem, regulatory, mucosal-associated invariant (MAIT), γδ (gd), tissue resident T cells, natural killer T cells, or any combination thereof. In certain embodiments, the T cells can be primary cells or cell lines derived from human, mouse, rat, or other mammals. If obtained from a mammal, a T cell can be obtained from numerous sources, including blood, bone marrow, lymph node, thymus, or other tissues or fluids. A T cell composition may be enriched or purified. T cell lines are well known in the art, some of which are described in Sandberg et al., Leukemia 21:230, 2000. In certain embodiments, the T cells lack endogenous expression of a TCRα gene, TCRβ gene, or both. Such T cells may naturally lack endogenous expression of TCRα and β chains, or may have been modified to block expression (e.g. T cells from a transgenic mouse that does not express TCR α and β chains or cells that have been manipulated to inhibit expression of TCR α and β chains) or to knockout a TCRα, chain, a TCRβ chain, or both genes.

In certain embodiments, host cells expressing a chimeric Tim protein of this disclosure on the cell surface are not T cells or cells of a T cell lineage, but cells that are progenitor cells, stem cells or cells that have been modified to express cell surface anti-CD3.

In certain embodiments, gene editing methods are used to modify the host cell genome to comprise a polynucleotide encoding a chimeric Tim receptor of the present disclosure. Gene editing, or genome editing, is a method of genetic engineering wherein DNA is inserted, replaced, or removed from a host cell's genome using genetically engineered endonucleases. The nucleases create specific double-stranded breaks at targeted loci in the genome. The host cell's endogenous DNA repair pathways then repair the induced break(s), e.g., by non-homologous ending joining (NHEJ) and homologous recombination Exemplary endonucleases useful for gene editing include a zinc finger nuclease (ZFN), a transcription activator-like effector (TALE) nuclease, a clustered regularly interspaced short palindromic repeats (CRISPR)/Cas nuclease system (e.g., CRISPR-Cas9), a meganuclease, or combinations thereof. Methods of disrupting or knocking out genes or gene expression in immune cells including B cells and T cells, using gene editing endonucleases are known in the art and described, for example, in PCT Publication Nos. WO 2015/066262; WO 2013/074916; WO 2014/059173; Cheong et al., Nat. Comm. 2016 7:10934; Chu et al., Proc. Natl Acad. Sci. USA 2016 113:12514-12519; methods from each of which are incorporated herein by reference in their entirety.

In certain embodiments, expression of an endogenous gene of the host cell is inhibited, knocked down, or knocked out. Examples of endogenous genes that may be inhibited, knocked down, or knocked out in a B cell include IGH, IGκ, IGλ, or any combination thereof. Examples of endogenous genes that may be inhibited, knocked down, or knocked out in a T cell include a TCR gene (TRA or TRB), an HLA gene (HLA class I gene or HLA class II gene), an immune checkpoint molecule (PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, TLA, KIR, LAG3, TIM3, A2aR, CD244/2B4, CD160, TIGIT, LAIR-1, or PVRIG/CD112R), or any combination thereof. Expression of an endogenous gene may be inhibited, knocked down, or knocked out at the gene level, transcriptional level, translational level, or a combination thereof. Methods of inhibiting, knocking down, or knocking out an endogenous gene may be accomplished, for example, by an RNA interference agent (e.g., siRNA, shRNA, miRNA, etc.) or an engineered endonuclease (e.g., CRISPR/Cas nuclease system, a zinc finger nuclease (ZFN), a Transcription Activator Like Effector nuclease (TALEN), a meganuclease), or any combination thereof. In certain embodiments, an endogenous B cell gene (e.g., IGH, IGκ, or IGλ) is knocked out by insertion of a polynucleotide encoding a chimeric Tim receptor of the present disclosure into the locus of the endogenous B cell gene, such as via an engineered endonuclease. In certain embodiments, an endogenous T cell gene (e.g., a TCR gene, an HLA gene, or an immune checkpoint molecule gene) is knocked out by insertion of a polynucleotide encoding a chimeric Tim receptor of the present disclosure into the locus of the endogenous T cell gene, such as via an engineered endonuclease.

In certain embodiments, a host cell may be genetically modified to express one type of chimeric Tim receptor. In other embodiments, a host cell may express at least two or more different chimeric Tim receptors.

The present disclosure also provides a composition comprising a population of chimeric Tim receptor modified host cells. In certain embodiments, the population of chimeric Tim receptor modified host cells may be a population of B cells, a population of T cells, a population of natural killer cells, a population of lymphoid precursor cells, a population of antigen presenting cells, a population of dendritic cells, a population of Langerhans cells, a population of myeloid precursor cells, a population of mature myeloid cells, or any combination thereof. Furthermore, a population of chimeric Tim receptor modified host cells of a particular cell type may be composed of one or more subtypes. For example, a population of B cells may be composed of chimeric Tim receptor modified naïve B cells, plasma cells, regulatory B cells, marginal zone B cells, follicular B cells, lymphoplasmacytoid cells, plasmablast cells, memory B cells, or any combination thereof. In another example, a population of T cells may be composed of chimeric Tim receptor modified CD4⁺ helper T cells, CD8⁺ effector (cytotoxic) T cells, naïve (CD45 RA+, CCR7+, CD62L+, CD27+, CD45RO−) T cells, central memory (CD45RO⁺, CD621L⁺, CD8⁺) T cells, effector memory (CD45RA+, CD45RO−, CCR7−, CD62L−, CD27−) T cells, T memory stem cells, regulatory T cells, mucosal-associated invariant T cells (MAIT), γδ (gd) cells, tissue resident T cells, natural killer T cells, or any combination thereof.

In certain embodiments, a population of host cells is composed of cells that each expresses the same chimeric Tim receptor(s). In other embodiments, a population of host cells is composed of a mixture of two or more subpopulation of host cells, wherein each subpopulation expresses a different chimeric Tim receptor or set of chimeric Tim receptors.

In certain embodiments, when preparing chimeric Tim receptor modified host cells, e.g., B cells or T cells, one or more growth factor cytokines that promotes proliferation of the host cells, e.g., B cells or T cells, may be added to the cell culture. The cytokines may be human or non-human. Exemplary growth factor cytokines that may be used to promote T cell proliferation include IL-2, IL-15, or the like. Exemplary growth factor cytokines that may be used to promote B cell proliferation include CD40L, IL-2, IL-4, IL-15, HL-21, BAFF, or the like.

Prior to genetic modification of the host cells with a chimeric Tim receptor vector, a source of host cells (e.g., T cells, B cells, natural killer cells, etc.) is obtained from a subject (e.g., whole blood, peripheral blood mononuclear cells, bone marrow, lymph node tissue, cord blood, thymus tissue, tissue from a site of infection, ascites, pleural effusion, spleen tissue), from which host cells are isolated using methods known in the art. Specific host cell subsets can be collected in accordance with known techniques and enriched or depleted by known techniques, such as affinity binding to antibodies, flow cytometry and/or immunomagnetic selection. After enrichment and/or depletion steps and introduction of a chimeric Tim receptor, in vitro expansion of the desired modified host cells can be carried out in accordance with known techniques, or variations thereof that will be apparent those skilled in the art.

Chimeric Tim receptors of the present disclosure confer cytotoxic activity to host cells expressing the chimeric Tim receptors that is specific for phosphatidylserine. Thus, upon binding phosphatidylserine exposed on the surface of a target cell, a host cell expressing a chimeric Tim receptor is capable of inducing apoptosis of the target cell. In certain embodiments, the host cell expressing the chimeric Tim receptor induces apoptosis of the target cell via: release of granzymes, perforins, granulysin, or any combination thereof; Fas ligand-Fas interaction: or both. In further embodiments, the chimeric Tim receptor further confers phosphatidylserine specific engulfment activity to host cells expressing the chimeric Tim receptor. In yet further embodiments, the host cell does not naturally exhibit an engulfment phenotype prior to modification with the chimeric Tim receptor.

Chimeric Tim receptors of the present disclosure may also be capable of costimulating T cells via at least one signaling pathway. In certain embodiments, chimeric Tim receptors provide costimulatory signals to T cells via at least two distinct signaling pathways (e.g., via the selected costimulatory signaling domain(s) in the chimeric Tim receptor). For example, a chimeric Tim receptor comprising a CD28 costimulatory signaling domain may be capable of providing a costimulatory signal via CD28 and Tim1. In certain embodiments, host immune cells expressing the chimeric Tim receptors exhibit reduction or inhibition of immune cell exhaustion. In certain embodiments, the host immune cell is a T cell or NK cells. In certain embodiments, exhausted T cells exhibit; (a) increased expression of PD-1, TIGIT, LAG3, TIM3, or any combination thereof; (b) decreased production of IFN-γ, IL-2, TNF-α, or any combination thereof; or both (a) and (b). In certain embodiments, exhausted NK cells exhibit; (a) increased expression of PD-1, NIKG2A, TIM3, or any combination thereof; (b) decreased production of IFN-γ, TNF-α, or both; or both (a) and (b).

In certain embodiments, host cells expressing the chimeric Tim receptors exhibit an enhanced effector response (e.g., tumor specific). In certain embodiments, the effector response is enhanced T cell proliferation, cytokine production (e.g., IFN-γ, IL-2, TNF-α), cytotoxic activity, persistence, or any combination thereof.

In certain embodiments host cells expressing the chimeric Tim receptors exhibit a reduced immunosuppressive response to phosphatidylserine. Phosphatidylserine is one of the primary apoptotic cell ligands that signal “eat me” to phagocytes. The removal of apoptotic cells by phagocytes generally reduces or prevents an inflammatory response via secretion of anti-inflammatory cytokines IL-10 and TGF-β and the decrease of secretion of inflammatory cytokines TNF-α, IL-1β, and IL-12. Thus, phosphatidylserine may act as an immunosuppressive signal during the clearance of apoptotic cells. In certain embodiments, upon binding phosphatidylserine, a chimeric Tim receptor modified host cell exhibits increased antigen-specific cytokine production (e.g., IFN-γ, IL-2, TNF-α), thereby reducing the immunosuppressive response to phosphatidylserine.

The expression of chimeric Tim receptors on host cells may be functionally characterized according to any of a large number of art-accepted methodologies for assaying host cell (e.g., T cell) activity, including determination of T cell binding, activation or induction and also including determination of T cell responses that are antigen-specific. Examples include determination of T cell proliferation, T cell cytokine release, antigen-specific T cell stimulation, CTL activity (e.g., by detecting ⁵¹Cr or Europium release from pre-loaded target cells), changes in T cell phenotypic marker expression, and other measures of T cell functions. Procedures for performing these and similar assays are may be found, for example, in Lefkovits (Immunology Methods Manual: the Comprehensive Sourcebook of Techniques, 1998). See, also, Current Protocols in Immunology; Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309 (1998) and references cited therein. Cytokine levels may be determined according to methods known in the art, including for example, ELISA, ELISPOT, intracellular cytokine staining, flow cytometry, and any combination thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like.

In certain embodiments, a chimeric Tim receptor modified host cell has a phagocytic index of about 20 to about 1,500 for a target cell. A “phagocytic index” is a measure of phagocytic activity of the transduced host cell as determined by counting the number of target cells or particles ingested per chimeric Tim receptor modified host cell during a set period of incubation of a suspension of target cells or particles and chimeric Tim receptor modified host cells in media. Phagocytic index may be calculated by multiplying [total number of engulfed target cells/total number of counted chimeric Tim receptor modified cells (e.g., phagocytic frequency)]×[average area of target cell or particle staining per chimeric Tim receptor⁺host cell×100 (e.g., hybrid capture)] or [total number of engulfed particles/total number of counted chimeric Tim receptor modified host cells]×[number of chimeric Tim receptor modified host cells containing engulfed particles/total number of counted chimeric Tim receptor cells]×100. In certain embodiments, a chimeric Tim receptor modified cell has a phagocytic index of about 30 to about 1,500; about 40 to about 1,500; about 50 to about 1,500; about 75 to about 1,500; about 100 to about 1,500; about 200 to about 1,500; about 300 to about 1,500; about 400 to about 1,500; about 500 to about 1,500; about 20 to about 1,400; about 30 to about 1,400; about 40 to about 1,400; about 50 to about 1,400; about 100 to about 1,400; about 200 to about 1,400; about 300 to about 1,400; about 400 to about 1,400; about 500 to about 1,400; about 20 to about 1,300; about 30 to about 1,300; about 40 to about 1,300; about 50 to about 1,300; about 100 to about 1,300; about 200 to about 1,300; about 300 to about 1,300; about 400 to about 1,300; about 500 to about 1,300; about 20 to about 1,200; about 30 to about 1,200; about 40 to about 1,200; about 50 to about 1,200; about 100 to about 1,200; about 200 to about 1,200; about 300 to about 1,200; about 400 to about 1,200; about 500 to about 1,200; about 20 to about 1,100; about 30 to about 1,100, about 40 to about 1,100; about 50 to about 1,100; about 100 to about 1,100; about 200 to about 1,100; about 300 to about 1,100; about 400 to about 1,100; or about 500 to about 1,100; about 20 to about 1,000; about 30 to about 1,000; about 40 to about 1,000; about 50 to about 1,000; about 100 to about 1,000; about 200 to about 1,000; about 300 to about 1,000; about 400 to about 1,000; or about 500 to about 1,000; about 20 to about 750; about 30 to about 750; about 40 to about 750; about 50 to about 750; about 100 to about 750; about 200 to about 750; about 300 to about 750; about 400 to about 750; or about 500 to about 750; about 20 to about 500; about 30 to about 500; about 40 to about 500; about 50 to about 500; about 100 to about 500; about 200 to about 500; or about 300 to about 500. In further embodiments, the incubation time is from about 2 hours to about 4 hours, about 2 hours, about 3 hours, or about 4 hours. In yet further embodiments, a chimeric Tim receptor modified cell exhibits phagocytic index that is statistically significantly higher than a cell transduced with truncated EGFR control. Phagocytic index may be calculated using methods known in the art and as further described in the Examples and PCT Application No. PCT/LTS2017/053553 (incorporated herein by reference in its entirety), including quantification by flow cytometry or fluorescence microscopy.

Host cells may be from an animal, such as a human, primate, cow, horse, sheep, dog, cat, mouse, rat, rabbit, guinea pig, pig, or a combination thereof. In a preferred embodiment, the animal is a human. Host cells may be obtained from a healthy subject or a subject having a disease associated with expression or overexpression of an antigen.

### PARP Inhibitors

Any suitable PARP inhibitor may be used in the compositions and methods of the disclosure. Exemplary PARP inhibitors include talazoparib, niraparib, rucaparib, olaparib (AZ 2281, KU59436), veliparib (ABT 888), CEP 9722, E7016, AG014699, MK4827, BMN-673, and Pamiparib (BGB3-290).

In a particular embodiment, the PARP inhibitor comprises niraparib. In a particular embodiment, the PARP inhibitor comprises talazoparib. In a particular embodiment, the PARP inhibitor comprises rucaparib. In a particular embodiment, the PARP inhibitor comprises olaparib. In a particular embodiment, the PARP inhibitor comprises veliparib. In a particular embodiment, the PARP inhibitor comprises CEP 9722. In a particular embodiment, the PARP inhibitor comprises E7016. In a particular embodiment, the PARP inhibitor comprises AG014699. In a particular embodiment, the PARP inhibitor comprises MK4827. In a particular embodiment, the PARP inhibitor comprises BMN-673. In a particular embodiment, the PARP inhibitor comprises Pamiparib.

### Methods of Treatment

A chimeric Tim receptor, a polynucleotides encoding a chimeric Tim receptor, a chimeric Tim receptor vector, or a host cell that expresses a chimeric Tim receptor, in combination with a PARP inhibitor according to any of the embodiments provided herein may be used in a method of treating a subject suffering from a disease, disorder or undesired condition. Embodiments of these methods include administering to a subject (i) a therapeutically effective amount of a pharmaceutical composition including one or more chimeric Tim receptors, polynucleotides encoding one or more chimeric Tim receptors, vectors comprising polynucleotides encoding one or more chimeric Tim receptors, or a population of host cells genetically modified to express one or more chimeric Tim receptors according to the present description; and (ii) a therapeutically effective amount of a pharmaceutical composition comprising a PARP inhibitor.

The chimeric Tim receptor compositions as described herein may be administered before PARP inhibitor therapy (e.g., 1 day to 30 days or more before the PARP inhibitor therapy), concurrently with PARP inhibitor therapy (on the same day), or after PARP inhibitor therapy (e.g., 1 day-30 days or more after the PARP inhibitor therapy). In certain embodiments, the chimeric Tim receptor modified cells are administered after administration of the one or more additional therapies. In further embodiments, the chimeric Tim receptor modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the PARP inhibitor. In still further embodiments, the chimeric Tim receptor modified cells are administered within 4 weeks, within 3 weeks, within 2 weeks, or within 1 week after administration of the PARP inhibitor therapy. Where the PARP inhibitor therapy involves multiple doses, the chimeric Tim receptor modified cells may be administered after the initial dose of the one or more additional therapies, after the final dose of the one or more additional therapies, or in between multiple doses of the one or more additional therapies.

Diseases that may be treated with cells expressing a chimeric Tim receptor as described in the present disclosure include cancer. Adoptive immune and gene therapies are promising treatments for various types of cancer (Morgan et al., Science 314:126, 2006; Schmitt et al., Hum. Gene Ther. 20:1240, 2009; June, J. Clin. Invest. 117:1466, 2007) and infectious disease (Kitchen et al., PLoS One 4:38208, 2009; Rossi et at, Nat. Biotechnol. 25:1444, 2007; Zhang et al., PLoS Pathog. 6:e1001018, 2010; Luo et al., J Mol. Med. 89:903, 2011).

A wide variety of cancers, including solid tumors and leukemias are amenable to the compositions and methods disclosed herein. Exemplary cancers that may be treated using the receptors, modified host cells, and composition described herein include adenocarcinoma of the breast, prostate, and colon; all forms of bronchogenic carcinoma of the lung; myeloid leukemia; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell). Additional types of cancers that may be treated using the receptors, modified host cells, and composition described herein include histiocytic disorders; malignant histiocytosis; leukemia; Hodgkin's disease; immunoproliferative small; non-Hodgkin's lymphoma; plasmacytoma; multiple myeloma; chronic myeloid leukemia (CML); acute myeloid leukemia (AML); plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment using the receptors, modified host cells, and composition described herein: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include angiokeratoma; angiolymphoid hyperplasia with eosinophilia; angioma sclerosing; angiomatosis; glomangioma; hemangioendothelioma; hemangioma; hemangiopericytoma; hemangiosarcoma: lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; neurofibromatosis; cervical dysplasia; and peritoneal cancer.

In some embodiments, compositions and methods of the present disclosure are useful to treat solid tumors. In some embodiments, the solid tumor cancer is breast cancer, ovarian cancer, colorectal cancer, fallopian cancer, peritoneal cancer, or prostate cancer. In some embodiments, the breast cancer is triple negative breast cancer. In some embodiments, the ovarian cancer is advanced ovarian cancer. In some embodiments, the prostate cancer is advanced prostate cancer. In some embodiments, the solid tumor cancer is melanoma. In some embodiments, the solid tumor cancer is lung cancer. In some embodiments, the lung cancer is non-small cell lung cancer.

In additional embodiments, the cancer is a Breast cancer (BRCA) mutated cancer. In particular embodiments, the cancer is a BRCA1 mutated cancer, a BRCA2 mutated cancer, or both.

A chimeric Tim receptor of the present disclosure may be administered to a subject in cell-bound form (e.g., gene therapy of target cell population). Thus, for example, a chimeric Tim receptor of the present disclosure may be administered to a subject expressed on the surface of T cells, Natural Killer Cells, Natural Killer T cells, B cells, lymphoid precursor cells, antigen presenting cells, dendritic cells, Langerhans cells, myeloid precursor cells, mature myeloid cells, including subsets thereof, or any combination thereof. In certain embodiments, methods of treating a subject comprise administering an effective amount of chimeric Tim receptor modified cells (i.e., recombinant cells that express one or more chimeric Tim receptors). The chimeric Tim receptor modified cells may be xenogeneic, syngeneic, allogeneic, or autologous to the subject.

Chimeric Tim receptor modified cells and PARP inhibitors may be administered in a manner appropriate to the disease or condition to be treated (or prevented) as determined by persons skilled in the medical art. An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently. The present disclosure provides pharmaceutical compositions comprising chimeric Tim receptor modified cells and a pharmaceutically acceptable carrier, diluent, or excipient. Suitable excipients include water, saline, dextrose, glycerol, or the like and combinations thereof. Other suitable infusion medium can be any isotonic medium formulation, including saline, Normosol R (Abbott). Plasma-Lyte A (Baxter), 5% dextrose in water, or Ringer's lactate.

A treatment effective amount of cells in a pharmaceutical composition is at least one cell (for example, one chimeric Tim receptor modified T cell) or is more typically greater than 10² cells, for example, up to 10⁶, up to 10⁷, up to 10⁸ cells, up to 10⁹ cells, up to 10¹⁰ cells, or up to 10¹¹ cells or more. In certain embodiments, the cells are administered in a range from about 10⁶ to about 10¹⁰ cells/m², preferably in a range of about 10⁷ to about 10⁹ cells/m². The number of cells will depend upon the ultimate use for which the composition is intended as well the type of cells included therein. For example, a composition comprising cells modified to contain a chimeric Tim receptor will comprise a cell population containing from about 5% to about 95% or more of such cells. In certain embodiments, a composition comprising chimeric Trim receptor modified cells comprises a cell population comprising at least 5%, 10%, 15%, 20%, 25%, 30%, 35%, 40%, 45%, 50%, 55%, 60%, 65%, 70%, 75%, 80%, 85%, 90%, 95% or more of such cells. For uses provided herein, the cells are generally in a volume of a liter or less, 500 mls or less, 250 mls or less, or 100 mls or less. Hence the density of the desired cells is typically greater than 1⁰⁴ cells/ml and generally is greater than 10⁷ cells/ml, generally 10⁸ cells/ml or greater. The cells may be administered as a single infusion or in multiple infusions over a range of time. Repeated infusions of chimeric Tim receptor modified cells may be separated by days, weeks, months, or even years if relapses of disease or disease activity are present. A clinically relevant number of immune cells can be apportioned into multiple infusions that cumulatively equal or exceed 10⁶, 10⁷, 10⁸, 10⁹, 10¹⁰, or 10¹¹ cells. A preferred dose for administration of a host cell comprising a recombinant expression vector as described herein is about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².

Chimeric Tim receptors and PARP inhibitors as described herein may be administered intravenously, intraperitoneally, intranasally, intratumorally, into the bone marrow, into the lymph node, and/or into cerebrospinal fluid.

In one aspect, methods of the present disclosure comprise conferring or enhancing phosphatidylserine-specific cytotoxic activity of a cell comprising introducing into a host cell a nucleic acid molecule encoding at least one chimeric Tim receptor or a chimeric Tim receptor vector according to any of the embodiments described herein; and expressing the at least one chimeric Tim receptor in the host cell, wherein the at least one chimeric Tim receptor enhances the phosphatidylserine-specific cytotoxic activity of the host cell as compared to a the host cell prior to modification to express a chimeric Tim receptor. In certain embodiments, the cytotoxic activity of the host cell is increased at least about 10%, 15%, 20%, 25%, 30%, 35%, 40%, 50%, 55%, 60%, 65%, 70%, 75%, 80%, 85%, 90%, 95%, 100%, 110%, 120%, 130%, 140%, 150%, 160%, 170%, 180%, 190%, 200% or more as compared to the host cell prior to modification with a nucleic acid molecule encoding a chimeric Tim receptor or a chimeric Tim receptor vector. In some embodiments, the host cell is an immune cell. In some embodiments, the host cell is a T cell or an NK cell. Methods of measuring cytotoxic activity of host cells, particularly immune cells such as T cells and NK cells, include a chromium (51Cr)-release assay, a β-gal or firefly luciferase release assay, flow cytometric methods of mediating target cell death and effector cell activity (see, e.g., Expert Rev. Vaccines, 2010, 9:601-616).

In certain embodiments, methods of the present disclosure comprise conferring or enhancing phosphatidylserine-specific cytotoxic activity of a cell further comprise conferring or enhancing phosphatidylserine-specific engulfment activity of the host cell expressing the at least one chimeric Tim receptor. In certain such embodiments, the host cell does not naturally exhibit an engulfment phenotype prior to modification with the chimeric Tim receptor. For example in certain such embodiments, the engulfment activity of the host cell is increased at least about 10%, 15%, 20%, 25%, 30%, 35%, 40%, 50%, 55%, 60%, 65%, 70%, 75%, 80%, 85%, 90%, 95%, 100%, 110%, 120%, 130%, 140%, 150%, 160%, 170%, 180%, 190%, 200% or more as compared to the host cell prior to modification to express the chimeric Tim receptor vector. In certain embodiments, the host cell does not naturally possess engulfment activity. In some embodiments, the host cell is an immune cell. In some embodiments, the host cell is a T cell or an NK cell. Methods of measuring engulfment activity of host cells include methods as described in PCT/US2017/053553 (incorporated herein by reference in its entirety).

In another aspect, a chimeric Tim receptor, a polynucleotide encoding a chimeric Tim receptor, a chimeric Tim receptor vector, or a host cell that expresses a chimeric Tim receptor according to any of the embodiments provided herein may be used in a method of enhancing effector function of the host cell. In certain embodiments, enhanced effector function comprises increased cytotoxic activity, increased antigen specific cytokine production (e.g., IFN-γ, IL-2, TNF-α, or any combination thereof), increased anti-apoptotic signaling, increased persistence, increased expansion, increased proliferation, or any combination thereof. In certain embodiments, the effector function of the host cell is enhanced at least about 10%, 15%, 20%, 25%, 30%, 35%, 40%, 50%, 55%, 60%, 65%, 70%, 75%, 80%, 85%, 90%, 95%, 100%, 110%, 120%, 130%, 140%, 150%, 160%, 170%, 180%, 190%, 200% or more as compared to a host cell that is not modified with a nucleic acid molecule encoding a chimeric Tim receptor or a chimeric Tim receptor vector. In some embodiments, the host cell is an immune cell. In certain embodiments, the host cell is a T cell or an NK cell.

In another aspect, host cells modified with chimeric Tim receptors of the present disclosure can be used in methods for inhibiting or reducing immune cell exhaustion. In some embodiments, the immune cell is a T cell or NK cell. In certain embodiments, reduced exhaustion in T cells comprises; (a) decreased expression of PD-1, TIGIT, LAG3, TIM3, or any combination thereof in T cells; (b) increased production of IFN-γ, IL-2, TNF-α, or any combination thereof in T cells; or both (a) and (b). In certain embodiments, reduced exhaustion in NK cells comprises; (a) decreased expression of PD-1, NKG2A, TIM3, or any combination thereof in NK cells; (b) increased production of IFN-γ, TNF-α, or both in NK cells; or both (a) and (b). In certain embodiments, the expression of an immune checkpoint molecule is decreased at least about 5%, 10%, 15%, 20%, 25%, 30%, 35%, 40%, 50%, 55%, 60%, 65%, 70%, 75%, 80%, 85%, 90%, 95%, or 100% in a host immune cell expressing the chimeric Tim receptor as compared to a host immune cell that is not modified with a nucleic acid molecule encoding a chimeric Tim receptor or a chimeric Tim receptor vector. In certain embodiments, the expression of the cytokine is increased at least about 5%, 10%, 15%, 20%, 25%, 30%, 35%, 40%, 50%, 55%, 60%, 65%, 70%, 75%, 80%, 85%, 90%, 95%, 100%, 110%, 120%, 130%, 140%, 150%, 160%, 170%, 180%, 190%, 200% or more in a host immune cell expressing the chimeric Tim receptor as compared to a host immune cell that is not modified with a nucleic acid molecule encoding a chimeric Tim receptor or a chimeric Tim receptor vector.

In another aspect, a chimeric Tim receptor, a polynucleotide encoding a chimeric Tim receptor, a chimeric Tim receptor vector, or a host cell that expresses a chimeric Tim receptor according to any of the embodiments provided herein may be used in a method of reducing an immunosuppressive response to phosphatidylserine in a host cell. In certain embodiments, the immunosuppressive response comprises secretion of anti-inflammatory cytokines (e.g., IL-10, TGF-β, or both), the decrease in secretion of inflammatory cytokines (e.g., TNF-α, HAL-1, and IL-12), or both. In certain embodiments, the immunosuppressive response of the host cell to phosphatidylserine is decreased at least about/5%, 10%, 15%, 20%, 25%, 30%, 35%, 40%, 50%, 55%, 60%, 65%, 70%, 75%, 80%, 85%, 90%, 95%, 100% as compared to a host cell that is not modified with a nucleic acid molecule encoding a chimeric Tim receptor or a chimeric Tim receptor vector. In some embodiments, the host cell is an immune cell. In certain embodiments, the host cell is a T cell or an NK cell.

In yet another aspect, a chimeric Tim receptor, a polynucleotide encoding a chimeric Tim receptor, a chimeric Tim receptor vector, or a host cell that expresses a chimeric Tim receptor according to any of the embodiments provided herein may be used in methods for eliminating target cells bearing surface exposed phosphatidylserine, e.g., for the elimination of cancer cells bearing surface presented phosphatidylserine. In certain embodiments, the target cells are damaged, stressed, apoptotic, necrotic cells (e.g., tumor cells) bearing surface exposed phosphatidylserine. In certain embodiments, host cells expressing chimeric Tim receptors clear damaged, stressed, apoptotic, or necrotic target cells bearing surface exposed phosphatidylserine via inducing apoptosis, or both inducing apoptosis and engulfment. Host cells expressing chimeric Tim receptors may be administered to a subject alone, or in combination with other therapeutic agents, including for example CAR-T cells, TCRs, antibodies, radiation therapy, chemotherapy, small molecules, oncolytic viruses, electropulse therapy, etc.

In another aspect, a chimeric Tim receptor, a polynucleotides encoding a chimeric Tim receptor, a chimeric Tim receptor vector, or a host cell that expresses a chimeric Tim receptor according to any of the embodiments provided herein may be used in methods to enhance the effect of a therapeutic agent that induces cellular stress, damage, necrosis, or apoptosis. Certain therapies, such as chemotherapy, radiation therapy, UV light therapy, electropulse therapy, adoptive cellular immunotherapy (e.g., CAR-T cells, TCRs) and oncolytic viral therapy, can induce cell damage or death to tumor cells, diseased cells, and cells in their surrounding environment. Cells expressing chimeric Tim receptors can be administered in combination with the cell damaging/cytotoxic therapy to bind to the phosphatidylserine moieties exposed on the outer leaflet of targeted cells and clear stressed, damaged, diseased, apoptotic, necrotic cells.

Chimeric Tim receptors and PARP inhibitors may be administered to a subject in combination with one or more additional therapeutic agents. Examples of therapeutic agents that may be administered in combination with a chimeric Tim compositions according to the present description include radiation therapy, adoptive cellular immunotherapy agent (e.g., recombinant TCR, enhanced affinity TCR, CAR, TCR-CAR, scTCR fusion protein, dendritic cell vaccine), antibody therapy, immune checkpoint molecule inhibitor therapy, UV light therapy, electric pulse therapy, high intensity focused ultrasound therapy, oncolytic virus therapy, or a pharmaceutical therapy, such as a chemotherapeutic agent, a therapeutic peptide, a hormone, an aptamer, antibiotic, anti-viral agent, anti-fungal agent, anti-inflammatory agent, a small molecule therapy, or any combination thereof. In certain embodiments, the chimeric Tim receptor modified host cells may clear stressed, damaged, apoptotic, necrotic, infected, dead cells displaying surface phosphatidylserine induced by the one or more additional therapeutic agents.

In certain embodiments, a vector comprises a polynucleotide encoding a chimeric Tim receptor and a polynucleotide encoding a cellular immunotherapy agent (e.g., chimeric antigen receptor, recombinant TCR, etc.). In certain embodiments, a single polynucleotide encoding the chimeric Tim receptor and cellular immunotherapy agent (e.g., CAR) is cloned into a cloning site and expressed from a single promoter, with the chimeric Tim receptor sequence and cellular immunotherapy agent (e.g., CAR) sequence separated from each other by an internal ribosomal entry site (IRES), furin cleavage site, or viral 2A peptide to allow for co-expression of multiple genes from a single open reading frame (e.g., a multicistronic vector). In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of SEQ ID NO: 12, 28, 29, or 30. An exemplary P2A peptide comprises an amino acid sequence of SEQ ID NO:13 or 31. An exemplary 2A peptide sequence comprises an amino acid sequence of SEQ ID NO:14. An exemplary F2A peptide sequence comprises an amino acid sequence of SEQ ID NO:15.

In certain embodiments, a polynucleotide encoding the chimeric Tim receptor and a polynucleotide encoding the cellular immunotherapy agent (e.g., CAR) binding protein are joined together into a single polynucleotide and then inserted into a vector. In other embodiments, a polynucleotide encoding the CER, and a polynucleotide encoding the CAR or TCR binding protein may be inserted separately into a vector in the same or different cloning sites, such that the expressed amino acid sequence produces a functional CER and CAR/or TCR. A vector that encodes a tandem expression cassette is referred to herein as a “tandem expression vector.”

In certain embodiments, a vector comprises a polynucleotide encoding a chimeric Tim receptor and a polynucleotide encoding a cellular immunotherapy agent (e.g., CAR). The polynucleotides encoding the chimeric Tim receptor and cellular immunotherapy agent (e.g., CAR) may be cloned sequentially into a vector at different cloning sites, with the chimeric Tim receptor and cellular immunotherapy agent (e.g., CAR) expressed under the regulation of different promoters.

In certain embodiments, a chimeric Tim receptor modified host cell may also be modified to co-express one or more small GTPases. Rho GTPases, a family of small (˜21 k Da) signaling G proteins and also a subfamily of the Ras superfamily, regulate actin cytoskeleton organization in various cell types and promote pseudopod extension and phagosome closure during phagocytosis (see, e.g., Castellano et al., 2000, J. Cell Sci. 113:2955-2961). Engulfment requires F-actin recruitment beneath tethered cells or particles, and F-actin rearrangement to allow membrane extension resulting in cell or particle internalization. RhoGTPases include RhoA, Rac1, Rac2, RhoG, and CDC42. Other small GTPases, such as Rap1, is involved in regulation of complement mediated phagocytosis. Co-expression of a small GTPase with the chimeric Tim receptor may promote target cell or particle internalization and/or phagosome formation by the host cell. In some embodiments, a recombinant nucleic acid molecule encoding a GTPase is encoded on a separate vector than the chimeric Tim receptor-containing vector. In other embodiments, a recombinant nucleic acid molecule encoding a GTPase is encoded on the same vector as the chimeric Tim receptor. The GTPase and chimeric Tim receptor may be expressed under the regulation of different promoters on the same vector (e.g., at different multiple cloning sites). Alternatively, the chimeric Tim receptor and GTPase may be expressed under the regulation of one promoter in a multicistronic vector. The polynucleotide sequence encoding the chimeric Tim receptor and the polynucleotide sequence encoding the small GTPase(s) may be separated from each other by an IRES or viral 2A peptide in a multicistronic vector. Exemplary 2A peptides include T2A (SEQ ID NO:12), P2A (SEQ ID NO: 13), E2A (SEQ ID NO: 14), F2A (SEQ ID NO:15). Examples of GTPases that may be co-expressed with a chimeric Tim receptor include Rac1, Rac2, Rab5 (also referred to as Rab5a), Rab7, Rap1, RhoA, RhoG, CDC42, or any combination thereof. In specific embodiments, the GTPase comprises or is a sequence that is at least 90%, at least 91%, at least 92%, at least 93%, at least 94%, at least 95%, at least 96%, at least 97%, at least 98%, at least 99%, at least 99.5%, or 100% identical to a Rac1 amino acid sequence of SEQ ID NO:17, a Rab5 amino acid sequence of SEQ ID NO: 18, a Rab7 amino acid sequence of SEQ ID NO:19, a Rap1 amino acid sequence of SEQ ID NO:20, a RhoA amino acid sequence of SEQ ID NO:21, a CDC42 amino acid sequence of SEQ ID NO:22, or any combination thereof.

In certain embodiments, a chimeric Tim receptor modified host cell may also be modified to co-express a cellular immunotherapy agent (e.g., CAR, TCR, etc.). In some embodiments, the cellular immunotherapy agent comprises a chimeric antigen receptor (CAR). CARs are recombinant receptors that generally comprise: an extracellular domain comprising a binding domain that binds to a target antigen; an intracellular signaling domain (e.g., comprising an ITAM containing intracellular signaling domain and optionally an intracellular costimulatory domain), and a transmembrane domain positioned between and connecting the extracellular domain and the intracellular signaling domain.

Binding domains suitable for use in CARs of the present disclosure include any antigen-binding polypeptide. A binding domain may comprise an antibody or antigen binding fragment thereof, including for example, a full length heavy chain, Fab fragment, Fab′, F(ab′)₂, sFv, VH domain, VL domain, dAb, VHH, CDR, and scFv. In certain embodiments, a CAR binding domain is murine, chimeric, human, or humanized.

In certain embodiments, the binding domain of the CAR targets a cancer or tumor antigen. Exemplary antigens that a CAR may target include CD138, CD38, CD33, CD123, CD72, CD79a, CD79b, mesothelin, PSMA, BCMA, ROR1, MUC-16, L1CAM, CD22, CD19, CD20, CD23, CD24, CD37, CD30, CA125, CD56, c-Met, EGFR, GD-3, HPV E6, HPV E7, MUC-1, HER2, folate receptor a, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, and GD2.

In certain embodiments, the extracellular domain of CARs provided in the present disclosure optionally comprises an extracellular, non-signaling spacer or linker domain. Where included, such a spacer or linker domain may position the binding domain away from the host cell surface to further enable proper cell to cell contact, binding, and activation. An extracellular spacer domain is generally located between the extracellular binding domain and the transmembrane domain of the CAR. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG4 hinge region is described in PCT Publication No. WO 2014/031687, which hinge region is incorporated herein by reference in its entirety. In a particular embodiment, an extracellular spacer domain comprises a modified IgG₄ hinge region having an amino acid sequence of SEQ ID NO:3.

Other examples of hinge regions that may be used in the CARs described herein include the hinge region from the extracellular regions of type 1 membrane proteins, such as CD8a, CD4, CD28 and CD7, which may be wild-type or variants thereof. In a particular embodiment, an extracellular spacer domain comprises a CD8a hinge region having an amino acid sequence of SEQ ID NO:70. In another particular embodiment, an extracellular spacer domain comprises a CD28 hinge region having an amino acid sequence of SEQ ID NO:32. In further embodiments, an extracellular spacer domain comprises all or a portion of an immunoglobulin Fc domain selected from: a CH1 domain, a CH2 domain, a C-3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NIKG2A, and NKG2D.

CARs of the present disclosure comprise a transmembrane domain that connects and is positioned between the extracellular domain and the intracellular signaling domain. The transmembrane domain ranges in length from about 15 amino acids to about 30 amino acids. The transmembrane domain is a hydrophobic alpha helix that transverses the host cell membrane and anchors the CAR in the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be selected from the same molecule as the extracellular domain or the intracellular signaling domain (e.g., a CAR comprises a CD28 costimulatory signaling domain and a CD28 transmembrane domain). In certain embodiments, the transmembrane domain and the extracellular domain are each selected from different molecules. In other embodiments, the transmembrane domain and the intracellular signaling domain are each selected from different molecules. In yet other embodiments, the transmembrane domain, the extracellular domain, and the intracellular signaling domain are each selected from different molecules.

Exemplary transmembrane domains for use in CARs of the present disclosure include a CD28, CD2, CD4, CD8a, CD5, CD3F, CD36, CD3ζ, CD9, CD16, CD22, CD25, CD27, CD33, CD37, D40, CD45, CD64, CD79A, CD793, CD80, CD86, CD95 (Fas), CD134 (OX40), CD137 (4-1BB), CD150 (SLAMF1), CD152 (CTLA4), CD1154 (CD40L), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH-2, NOTCH3, NOTCH4, PTCH2, ROR2, Ryk, Slp76, SIRPα, pTα, TCRα, TCRβ, TIM3, TRIM, LPA5, and Zap70 transmembrane domain. An exemplary CD28 transmembrane domain comprises an amino acid sequence of SEQ ID NO:7. In a particular embodiment, a transmembrane domain comprises a CD8a transmembrane domain having an amino acid sequence of SEQ ID NO:33.

The intracellular signaling domain of a CAR is an intracellular effector domain and is capable of transmitting functional signals to a cell in response to binding of the extracellular domain of the CAR to a target molecule (e.g., cancer antigen) and activates at least one of the normal effector functions or responses of the immune cell, e.g., T cell engineered to express the CAR. In some embodiments, the CAR induces a function of a T cell such as cytolytic activity or T helper activity, such as secretion of cytokines or other factors. The intracellular signaling domain may be any portion of an intracellular signaling molecule that retains sufficient signaling activity. In some embodiments, the intracellular signaling domain is obtained from an antigen receptor component (e.g., TCR) or costimulatory molecule. In some embodiments, a full length intracellular signaling domain of an antigen receptor or costimulatory molecule is used. In some embodiments, a truncated portion of an intracellular signaling domain of an antigen receptor or costimulatory molecule is used, provided that the truncated portion retains sufficient signal transduction activity. In further embodiments, an intracellular signaling domain is a variant of a full length or truncated portion of an intracellular signaling domain of an antigen receptor co stimulatory molecule, provided that the variant retains sufficient signal transduction activity (i.e., is a functional variant).

In certain embodiments, the intracellular signaling domain of a CAR comprises an immunoreceptor tyrosine-based activation motif (ITAM) containing signaling domain. An ITAM containing signaling domain generally contains at least one (one, two, three, four, or more) ITAMs, which refer to a conserved motif of YXXL/I-X₆₋₈-YXXL/I. An ITAM containing signaling domain may initiate T cell activation signaling following antigen binding or ligand engagement. ITAM-signaling domains include, for example, intracellular signaling domains of CD3γ, CD3δ, CD3ε, CD3ζ, CD5, CD22, CD79a, CD278 (ICOS), DAP12, FcRγ, and CD66d. Exemplary CD3ζ signaling domains that may be used in CARs of the present disclosure comprise an amino acid sequence of SEQ ID NO:27 or 5.

CAR intracellular signaling domains optionally comprise a costimulatory signaling domain, which, when activated in conjunction with a primary or classic (e.g., ITAM-driven) activation signal, promotes or enhances T cell response, such as T cell activation, cytokine production, proliferation, differentiation, survival, effector function, or combinations thereof. Costimulatory signaling domains for use in CARs include, for example, CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, NKG2C, SLP76, TRIM, ZAP70, or any combination thereof. In a particular embodiment, the costimulatory signaling domain comprises a OX40, CD2, CD27, CD28, ICAM-1, LFA-1 (CD11a/CD18), ICOS ((CD278), or 4-1BB (CD137) signaling domain. Exemplary CD28 costimulatory signaling domains that may be used in CARs of the present disclosure comprise an amino acid sequence of SEQ ID NO:26 or 4. An exemplary 4-1BB costimulatory signaling domain comprises an amino acid sequence of SEQ ID NO:122. In certain embodiments, a CAR comprises one, two, or more costimulatory signaling domains.

In some embodiments, CARs are recombinant receptors composed of an scFv binding domain derived from an antibody, a transmembrane domain, and an intracellular signaling domain(s). In some embodiments, the intracellular signaling domain(s) are derived from a TCR.

In certain embodiments, a chimeric antigen receptor comprises an amino acid sequences derived from any mammalian species, including humans, primates, cows, horses, goats, sheep, dogs, cats, mice, rats, rabbits, guinea pigs, pigs, transgenic species thereof, or any combination thereof. In certain embodiments, chimeric antigen receptor is murine, chimeric, human, or humanized.

In certain embodiments, a CAR is a first generation CAR, a second generation CAR, or a third generation CAR. A first generation CAR generally has an intracellular signaling domain comprising an intracellular signaling domain of CD3ζ, FcγRI, or other ITAM-containing activating domain to provide a T cell activation signal. Second generation CARs further comprise a costimulatory signaling domain (e.g., a costimulatory signaling domain from an endogenous T cell costimulatory receptor, such as CD28, 4-1BB, or ICOS). Third generation CARs comprise an ITAM-containing activating domain, a first costimulatory signaling domain and a second costimulatory signaling domain.

In some embodiments, one or more of the extracellular domain, the binding domain, the linker, the transmembrane domain, the intracellular signaling domain, or the costimulatory domain comprises junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane: domain of a fusion protein may have one or more junction amino acids at the amino-terminal end, carboxy-terminal end, or both.

Exemplary binding domain, extracellular spacer, transmembrane, and intracellular signaling domain sequences for use in CARs of the present disclosure are set forth in Table 11.

TABLE 11

| | | SEQ ID |
|---|---|---|
| Name | Amino Acid Sequence | NO: |
| Flexible linker | GGGGSGGGGSGGGGS | 89 |
| Flexible linker | GTGGSGGTGSGTGTS | 90 |
| IgG4 hinge | ESKYGPPCPPCP | 91 |
| region (short) | | |
| CD8a hinge | TTTPAPRPPTPAPTIASQPLSLRPEACRPAAGGAVHTR | 92 |
| region | GLDFACD | |
| CD28 hinge | IEVMYPPPYLDNEKSNGTIIHVKGKHLCPSPLFPGPSKP | 93 |
| region | | |
| CD8a | IYIWAPLAGTCGVLLLSLVITLYC | 94 |
| transmembrane | | |
| region | | |
| CD28 | FWVLVVVGGVLACYSLLVTVAFIIFWV | 95 |
| transmembrane | | |
| region | | |
| wildtype CD3ζ | RVKFSRSADAPAYQQGQNQLYNELNLGRREEYDVLD | 96 |
| signaling | KRRGRDPEMGGKPQRRKNPQEGLYNELQKDKMAEA | |
| domain | YSEIGMKGERRRGKGHDGLYQGLSTATKDTYDALHM | |
| | QALPPR | |
| variant CD3ζ | RVKFSRSADAPAYQQGQNQLYNELNLGRREEYDVLD | 97 |
| signaling | KRRGRDPEMGGKPRRKNPQEGLYNELQKDKMAEAY | |
| domain | SEIGMKGERRRGKGHDGLYQGLSTATKDTYDALHMQ | |
| | ALPPR | |
| wildtype CD28 | RSKRSRLLHSDYMNMTPRRPGPTRKHYQPYAPPRDFA | 98 |
| costimulatory | AYRS | |
| signaling | | |
| domain | | |
| variant CD28 | RSKRSRGGHSDYMNMTPRRPGPTRKHYQPYAPPRDF | 99 |
| costimulatory | AAYRS | |
| signaling | | |
| domain with | | |
| L186G/L187G | | |
| substitutions | | |
| with positions in | | |
| reference to full | | |
| length protein | | |
| 4-1BB | KRGRKKLLYIFKQPFMRPVQTTQEEDGCCRFPEEEE | 100 |
| costimulatory | GGCEL | |
| signaling | | |
| domain | | |
| GMCSFR_Signal | MLLVTSLLLCELPHPAFLLIP | 117 |
| Peptide | | |

In certain embodiments, a chimeric Tim receptor modified host cell co-expresses a recombinant TCR. Recombinant TCR proteins include “traditional” TCRs composed of a heterodimer of a chain polypeptide and β chain polypeptide or a heterodimer of a γ chain polypeptide and a δ chain polypeptide, binding fragments and fusion proteins thereof, including for example, single chain TCRs, single domain TCRs, soluble TCR fusion TCR proteins, and TCR fusion constructs (TRuC™). In certain embodiments, a tandem expression cassette comprises a polynucleotide encoding a recombinant TCR beta chain comprising a TCR beta variable region and a TCR beta constant region, and a polynucleotide encoding a recombinant TCR alpha chain comprising a TCR alpha variable region and a TCR alpha constant region. In certain embodiments, a recombinant TCR is an enhanced affinity TCR. In one embodiment, a recombinant TCR is an enhanced affinity TCR.

In certain embodiments, a recombinant TCR binding protein is a single chain TCR (scTCR) comprising a Vα joined to a Vβ by a flexible linker. In some embodiments, a scTCR comprises a Vα-linker-Vβ polypeptide. In other embodiments, a scTCR comprises a Vβ-linker-Vα polypeptide.

In certain embodiments, a chimeric Tim receptor modified host cell may also be modified to co-express a single chain TCR (scTCR) fusion protein A scTCR fusion protein comprises a binding domain comprising a scTCR (a TCR Vα domain linked to a ICR Vβ domain), an optional extracellular spacer, a transmembrane domain, and an intracellular component comprising a single intracellular signaling domain providing an T cell activation signal (e.g., a CD3ζ ITAM-containing activating domain) and optionally a costimulatory signaling domain (see, Aggen et al., 2012, Gene Ther. 19:365-374; Stone et al., Cancer Immunol. Immunother. 2014, 63:1163-76).

In certain embodiments, a chimeric Tim receptor modified host cell may also be modified to co-express a T cell receptor-based chimeric antigen receptor (TCR-CAR). A TCR-CAR is a heterodimeric fusion protein generally comprising a soluble TCR (a polypeptide chain comprising a Vα domain and Cα domain and a polypeptide chain comprising a Vβ domain and a Cβ domain) wherein the VβCβ polypeptide chain is linked to a transmembrane domain and an intracellular signaling component (e.g., an ITAM-containing activating domain and optionally a costimulatory signaling domain) (see, e.g., Walseng et al., 2017 Scientific Reports 7:10713).

In certain embodiments, an engineered host cell that co-expresses a chimeric Tim receptor and a cellular immunotherapy agent (e.g., CAR, TCR, etc.) comprises a recombinant nucleic acid encoding the chimeric Tim receptor and a recombinant nucleic acid molecule encoding the cellular immunotherapy agent on separate vectors within the engineered host cell.

In some embodiments, an engineered host cell that co-expresses a chimeric Tim receptor and a cellular immunotherapy agent (e.g., CAR, TCR, etc.) comprises a recombinant nucleic acid encoding the chimeric Tim receptor and a recombinant nucleic acid molecule encoding the cellular immunotherapy agent on the same vector as the chimeric Tim receptor within an engineered host cell. The chimeric Tim receptor and cellular immunotherapy agent may be expressed tinder the regulation of different promoters on the same vector (e.g., at different multiple cloning sites). Alternatively, the chimeric Tim receptor and cellular immunotherapy agent may be expressed under the regulation of one promoter in a multicistronic vector (e.g., tandem expression vector). The polynucleotide sequence encoding the chimeric Tim receptor and the polynucleotide sequence encoding the cellular immunotherapy agent may be separated by an IRES or viral 2A peptide in a multicistronic vector.

Tandem expression cassettes, tandem expression vectors, and engineered host cells comprising the same are described in International Application Publication No. WO2019/191339, which is incorporated herein by reference in its entirety.

Host cells expressing chimeric Tim receptors may be administered to a subject alone, or in combination with other therapeutic agents, including for example CAR-T cells, TCRs, antibodies, radiation therapy, chemotherapies, small molecules, oncolytic viruses, electropulse therapy, etc.

In certain embodiments, the chimeric Tim receptor and adoptive cellular immunotherapy agent (e.g., a CAR, TCR-CAR, TCR, etc. described above) are administered to the subject in the same host cell or different host cells. In certain embodiments, the chimeric Tim receptor and adoptive cellular immunotherapy agent are expressed in the same host cell from the same vector or from separate vectors. In certain embodiments, the chimeric Tim receptor and adoptive cellular immunotherapy agent are expressed in the same host cell from a multicistronic vector. In certain embodiments, the chimeric Tim receptor is expressed in the same host cell type as the adoptive cellular immunotherapy agent (e.g., the chimeric Tim receptor is expressed CD4 T cells and the CAR/or TCR is expressed in CD4 T cells, or the chimeric Tim receptor is expressed CD8 T cells and the CAR/or TCR is expressed in CD8 T cells). In other embodiments, the chimeric Tim receptor is expressed in a different host cell type as the adoptive immunotherapy agent (e.g., the chimeric Tim receptor is expressed CD4 T cells and the CAR/or TCR is expressed in CD8 T cells). Cellular immunotherapy compositions comprising a combination of immune cells or cellular subsets engineered with chimeric Tim receptors and a cellular immunotherapy agent (e.g., CAR, TCR, etc.), methods of making, and methods of use are described in PCT International Publication No. WO2019/191340, which is incorporated herein by reference in its entirety.

Exemplary antigens that a recombinant TCR, enhanced affinity TCR, CAR, TCR-CAR, or scTCR fusion protein may target include WT-1, mesothelin, MART-1, NY-ESO-1, MAGE-A3, HPV E7, survivin, α Fetoprotein, and a tumor-specific neoantigen.

CARs of the present disclosure may target a variety of antigens, including a viral antigen, bacterial antigen, fungal antigen, parasitic antigen, tumor antigen, autoimmune disease antigen. Exemplary antigens that a CAR may target include CD138, CD38, CD33, CD123, CD72, CD79a, CD79b, mesothelin, PSMA, BCMA, ROR1, MUC-16, L1CAM, CD22, CD19, CD20, CD23, CD24, CD37, CD30, CA125, CD56, c-Met, EGFR, GD-3, HPV E6, HPV E7, MUC-1, HER2, folate receptor a, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor, ECFRviii, VEGFR-2, LGR5, SSX2, AKAP-4, FLT3, fucosyl GM1, GM3, o-acetyl-CD2, and GD2.

Radiation therapy includes external beam radiation therapy (e.g., conventional external beam radiation therapy, stereotactic radiation, 3-dimensional conformal radiation therapy, intensity-modulated radiation therapy, volumetric modulated arc therapy, particle therapy, proton therapy, and auger therapy), brachytherapy, systemic radioisotope therapy, intraoperative radiotherapy, or any combination thereof.

Exemplary antibodies for use in conjunction with the chimeric Tim compositions described herein include rituxmab, pertuzumab, trastuzumab, alemtuzumab, Ibritumomab tiuxetan, Brentuximab vedotin, cetuximab, bevacizumnab, abciximab, adalimumab, alefacept, basilizimab, belimumab, bezlotoxumab, canakinumab, certolizumab pegol, daclizumab, denosumab, efalizumab, golimumab, olaratumab, palivizumab, panitumumab, and tocilizumab.

Exemplary inhibitors of immune checkpoint molecules that may be for use in conjunction with the chimeric Tim compositions described herein include checkpoint inhibitors targeting PD-1L, PD-L2, CD80, CD86, B7-H3, 137-1H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/234, CD160, TIGIT, LAIR-1, PVRIG/CD112R, or any combination thereof. In certain embodiments, an immune checkpoint inhibitor may be an antibody, a peptide, an RNAi agent, or a small molecule. An antibody specific for CTLA-4 may be ipilimumab or tremelimumab. An antibody specific for PD-1 may be pidilizumab, nivolumab, or pembrolizumab. An antibody specific for PD-L1 may be durvalumab, atezolizumab, or avelumab.

Exemplary chemotherapeutics for use in conjunction with the chimeric Tim receptor compositions described herein may include an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

A chemotherapeutic includes non-specific cytotoxic agents that inhibit mitosis or cell division, as well as molecularly targeted therapy that blocks the growth and spread of cancer cells by targeting specific molecules that are involved in tumor growth, progression, and metastasis (e.g., oncogenes). Exemplary non-specific chemotherapeutics for use in conjunction with the expression cassette compositions described herein may include an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), hypomethylating agent, and a DNA repair inhibitor.

Examples of chemotherapeutic agents considered for use in combination therapies contemplated herein include vemurafenib, dabrafenib, trametinib, cobimetinib, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome)), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), fdabra tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), ibrutinib, venetoclax, crizotinib, alectinib, brigatinib, ceritinib, and vinorelbine (Navelbine®).

Exemplary alkylating agents for use in combination therapies contemplated herein include nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilnostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents for use in combination therapies contemplated herein include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary platinum based agents for use in combination therapies contemplated herein include carboplatin, cisplatin, oxaliplatin, nedaplatin, picoplatin, satraplatin, phenanthriplatin, and triplatin tetranitrate.

Exemplary hypomethylating agents for use in combination therapies include azacitidine and decitabine.

Exemplary molecularly targeted inhibitors for use in conjunction with the chimeric Tim receptor compositions described herein include small molecules that target molecules involved in cancer cell growth and survival, including for example, receptor tyrosine kinase inhibitors, RAF inhibitors, BCL-2 inhibitors, ABL inhibitors, TRK inhibitors, c-KIT inhibitors, c-MET inhibitors, CDK4/6 inhibitors, FAK inhibitors, FGFR inhibitors, FLT3 inhibitors, IDH1 inhibitors, ID-12 inhibitors, PDGFRA inhibitors, and RET inhibitors

Exemplary molecularly targeted therapy includes hormone antagonists, signal transduction inhibitors, gene expression inhibitors (e.g., translation inhibitors), apoptosis inducers, angiogenesis inhibitors (e.g., a VEGF pathway inhibitor), tyrosine kinase inhibitors (e.g., an EGF/EGFR pathway inhibitor), growth factor inhibitors, GTPase inhibitors, serine/threonine kinase inhibitors, transcription factor inhibitors, inhibitors of driver mutations associated with cancer, B-Raf inhibitors, RAF inhibitors, a MEK inhibitors, mTOR inhibitors, adenosine pathway inhibitors, EGFR inhibitors, PI3K inhibitors, BCL2 inhibitors, VEGFR inhibitors, MET inhibitors, MYC inhibitors, BCR-ABL inhibitors, ABL inhibitors, HER2 inhibitors, H-RAS inhibitors, K-RAS inhibitors, PDGFR inhibitors, ALK inhibitors, ROS1 inhibitors, BTK inhibitors, TRK inhibitors, c-KIT inhibitors, c-MET inhibitors, CDK4/6 inhibitors, FAK inhibitors, FGFR inhibitors, FLT3 inhibitors, IDH1 inhibitors, IDH2 inhibitors, PARP inhibitors, PARP inhibitors, PDGFRA inhibitors, and RET inhibitors. In certain embodiments, use of molecularly targeted therapy comprises administering a molecularly targeted therapy specific for the molecular target to a subject identified as having a tumor that possesses the molecular target (e.g., driver oncogene). In certain embodiments, the molecular target has an activating mutation. In certain embodiments, use of chimeric Tim receptor modified cells in combination with a molecularly targeted inhibitor increases the magnitude of anti-tumor response, the durability of anti-tumor response, or both. In certain embodiments, a lower than typical dose of molecularly targeted therapy is used in combination with chimeric Tim receptor modified cells.

Exemplary angiogenesis inhibitors include, without limitation A6 (Angstrom Pharmaceuticals), ALT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP 02) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQule), ASA404 (Novartis/Antisoma), Atiprimod (Callisto Pharmaceuticals), ATN-161 (Attenuon), AV-412 (Aveo Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Boehringer Ingelheim Pharmaceuticals), BIBW 2992 (Boehringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharmaceuticals), Combretastatin (Oxigene), CP-751,871 (Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfiram (Antabuse), E7820 (Eisai Limited), E7080 (Eisai Limited), EMD 121974 (Cilengitide) (EMD Pharmaceuticals), ENMD-1198 (EntreMed), ENMD-2076 (EntreMed), Endostar (Simcere), Erbitux (ImClone/Bristol-Myers Squibb), EZN-2208 (Enzon Pharmaceuticals), EZN-2968 (Enzon Pharmaceuticals), GC1008 (Genzyme), Genistein, GSK1363089 (Foretinib) (GlaxoSmithKline), GW786034 (Pazopanib) (GlaxoSmithKline), GT-111 (Vascular Biogenics Ltd.), IMC-1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostat) (Novartis), Lucentis (Ranibizumab) (Genentech/Novartis), LY317615 (Enzastaurin) (Eli Lilly and Company), Macugen (Pegaptanib) (Pfizer), MED1522 (Abegrin) (Medimmune), MLN518 (Tandutinib) (Millennium), Neovastat (AE941/Benefin) (Aeterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCID (2ME2) (EntreMed), PF-02341066 (Pfizer), PF-04554878 (Pfizer), Pf-88 (Progen Industries/Medigen Biotechnology), PKC412 (Novartis), Polyphenon E (Green Tea Extract) (Polypheno E International, Inc.), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230 (Pasireotide) (Novartis), Squalamine (Genaera), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TKI258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exelixis), XL647 (Exelixis), XL784 (Exelixis), XL820 (Exelixis), XL999 (Exelixis), ZD6474 (AstraZeneca), Vorinostat (Merck), and ZSTK474.

Exemplary B-Raf inhibitors include vemurafenib, dabrafenib, and encorafenib.

Exemplary MEK inhibitors include binimetinib, cobimetinib, refametinib, selumetinib, and trametinib.

Exemplary 3TK inhibitors include ibrutinib, Loxo-305, tirabrutinib, GDC-0853, acalabrutinib, ONO-4059, spebrutinib, BGB-3111, H1M71224, and M7583.

Exemplary TRK inhibitors include entrectinib, larotrectinib, CH7057288, ONO-7579, LOXO-101, lestaurtinib, and LOXO-195.

Exemplary c-KIT inhibitors include imatinb, sunitinb, and ponatinib.

Exemplary c-MET inhibitors include capmatinib, crizotinib, tivantinib, onartuzumab, INCB28060, AMiG-458, savolitinib, and tepotinib.

Exemplary CDK4/6 inhibitors include palbociclib, ribociclib, abemaciclib, and trilaciclib.

Exemplary FAK inhibitors include defactinib, GSK2256098, 131853520, and PF-00562271.

Exemplary FGFR inhibitors include erdafitinib, pemigatinib, infigratinib, rogaratinib, AZD4547, BGJ398, FP-1039, and ARQ 087.

Exemplary FLT-3 inhibitors include quizartinib, crenolanib, gilteritinib, midostaurin, and lestaurtinib.

Exemplary IDH1 inhibitors include ivosidenib, BAY-1436032, and AGI-5198.

An exemplary IDH2 inhibitor includes enasidenib.

Exemplary PDGFRA inhibitors include imatinib, regorafenib, crenolanib, and olaratumab.

Exemplary pan-RAF inhibitors include belvarafenib, LXH254, LY3009120, INT-152, and HM95573.

Exemplary RET inhibitors include lenvatinib, alectinib, vandetanib, cabozantinib, BLU-667, and LOXO-292.

Exemplary ROS1 inhibitors include ceritinib, lorlatinib, entrectinib, crizotinib, TPX-0005, and DS-6051b.

Exemplary Vascular Endothelial Growth Factor (VEGF) receptor inhibitors include, but are not limited to, Bevacizumab (Avastin®), axitinib (Inlyta®); Brivanib alaninate (BMS-582664, (S)-((R)-1-(4-(4-Fluoro-2-methyl-11H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-(3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3α,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

Exemplary EGF pathway inhibitors include, without limitation tyrphostin 46, EKB-569, erlotinib (Tarceva®), gefitinib (Iressa®), erbitux, nimotuzumab, lapatinib (Tykerb®), cetuximab (anti-EGFR mAb), ¹⁸⁸Re-labeled nimotuzumab (anti-EGER mAb), and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980. Exemplary EGFR antibodies include, but are not limited to, Cetuximab (Erbitux®); Panitumumab (Vectibix®); Matuzumab (EMD-72000); Trastuzumab (Herceptin®); Nimotuzumab (hR3); Zalutumumab, TheraCIM h-R3; MDX0447 (CAS 339151-96-1); and ch806 (mAb-806, CAS 946414-09-1). Exemplary Epidermal growth factor receptor (EGFR) inhibitors include, but not limited to, Erlotinib hydrochloride (Tarceva®); ceritinib; brigatinib; osimeritinib; icotinib; Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1-H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-[4-[[(1R)-1-Phenylethyl]amino]-7H-pyrrolo[2,3-d]pyrimidin-6-yl]-phenol (PKI166, CAS 187724-61-4): rociletinib.

Exemplary mTOR inhibitors include, without limitation, rapamycin (Rapamune®), and analogs and derivatives thereof, SDZ-RAD; Temsirolimus (Torisel®; also known as CCI-779); Ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Evenolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1).

Exemplary Phosphoinositide 3-kinase (PI3K) inhibitors include, but are not limited to, duvelisib, idelalisib, 4-[2-(1H-Indazol-1-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6). Exemplary Protein Kinase B (PKB) or AKT inhibitors include, but are not limited to. 8-[4-(1-Aminocyclobutyl)phenyl]-9-phenyl-1,2,4-triazolo[3,4-f][1,6]naphthyridin-3(2H)-one (NK-2206, CAS 1032349-93-1); Perifosine (KRX0401); 4-Dodecyl-N-1,3,4-thiadiazol-2-yl-benzenesulfonamide (PHT-427, CAS 1191951-57-1); 4-[2-(4-Amino-1,2,5-oxadiazol-3-yl)-1-ethyl-7-[(3S)-3-piperidinylmethoxy]-1H-imidazo[4,5-c]pyridin-4-yl]-2-methyl-3-butyn-2-ol (GSK690693, CAS 937174-76-0); 8-(1-Hydroxyethyl)-2-methoxy-3-[(4-methoxyphenyl)methoxy]-6H-dibenzo[b,d]pyran-6-one (palomid 529, P529, or SG-00529); Tricirbine (6-Amino-4-methyl-8-(β-D-ribofuranosyl)-4H,8H-pyrrolo[4,3,2-de]pyrimido[4,5-c]pyridazine); (αS)-α-[[[5-(β-Methyl-1H-indazol-5-yl)-3-pyridinyl]oxy]methyl]-benzeneethanamine (A674563, CAS 552325-73-2); 4-[(4-Chiorophenyl)methyl]-1-(7H-pyrrolo[2,3-d]pyrimidin-4-yl)-4-piperidinamine (CCT128930, CAS 885499-61-6); 4-(4-Chlorophenyl)-4-[4-(1H pyrazol-4-yl)phenyl]-piperidine (AT7867, CAS 857531-00-1); and Archexin (RX-0201, CAS 663232-27-7).

In certain embodiments, a tyrosine kinase inhibitor used in combination with chimeric Tim receptor modified cells is an anaplastic lymphoma kinase (ALK) inhibitor. Exemplary ALK inhibitors include crizotinib, ceritinib, alectinib, brigatinib, dalantercept, entrectinib, and lorlatinib.

In certain embodiments where chimeric Tim receptor modified cells are administered in combination with one or more additional therapies, the one or more additional therapies may be administered at a dose that might otherwise be considered subtherapeutic if administered as a monotherapy. In such embodiments, the chimeric Tim receptor composition may provide an additive or synergistic effect such that the one or more additional therapies can be administered at a lower dose. Combination therapy includes administration of a chimeric Tim receptor compositions as described herein before an additional therapy (e.g., 1 day to 30 days or more before the additional therapy), concurrently with an additional therapy (on the same day), or after an additional therapy (e.g., 1 day-30 days or more after the additional therapy). In certain embodiments, the chimeric Tim receptor modified cells are administered after administration of the one or more additional therapies. In further embodiments, the chimeric Tim receptor modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the one or more additional therapies. In still further embodiments, the chimeric Tim receptor modified cells are administered within 4 weeks, within 3 weeks, within 2 weeks, or within 1 week after administration of the one or more additional therapies. Where the one or more additional therapies involves multiple doses, the chimeric Tim receptor modified cells may be administered after the initial dose of the one or more additional therapies, after the final dose of the one or more additional therapies, or in between multiple doses of the one or more additional therapies.

In certain embodiments, methods of the present disclosure include a depletion step. A depletion step to remove chimeric Tim receptors from the subject may occur after a sufficient amount of time for therapeutic benefit in order to mitigate toxicity to a subject. In such embodiments, the chimeric Tim receptor vector may include an inducible suicide gene, such as iCASP9, inducible Fas, or HSV-TK. Similarly, a chimeric Tim receptor vector may be designed for expression of a known cell surface antigen such as CD20 or truncated EGFR (SEQ LD NO: 16) that facilitates depletion of transduced cells through infusion of an associated monoclonal antibody (mAb), for example, Rituximab for CD20 or Cetuximab for EGFR. Alemtuzumab, which targets CD52 present on the surface of mature lymphocytes, may also be used to deplete transduced B cells, T cells, or natural killer cells.

Subjects that can be treated by the compositions and methods of the present disclosure include animals, such as humans, primates, cows, horses, sheep, dogs, cats, mice, rats, rabbits, guinea pigs, or pigs. The subject may be male or female, and can be any suitable age, including infant, juvenile, adolescent, adult, and geriatric subjects.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

### Example 1

### Evaluating Tim-CER-T Cells for Synthetic Lethal PARP Inhibitor Synergisms in Ovarian Cancer

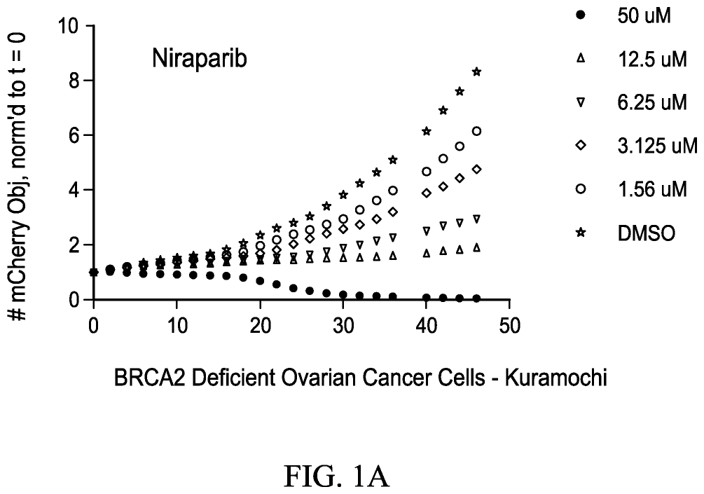

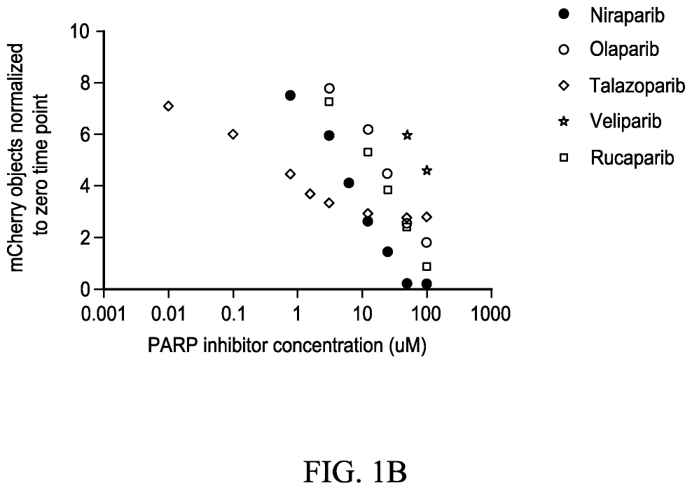

Kuramochi ovarian cancer cells expressing an mCherry-NLS plasmid were incubated with 0.78 to 100 uM of the PARP inhibitor Niraparib. The number of Kuramochi cells/well was tracked via imaging. At t=50, the number of Kuramochi cells in culture decreases monotonically with increasing Niraparib concentration. The results are shown in FIG. 1A.

Similar experiments were performed at various doses of the PARP inhibitors Niraparib, Olaparib, Talazoparib, Veliperib, and Rucaparib, and the number of Kuramochi cells was plotted at t=50 h. Talazoparib and Niraparib exhibit the most sensitive EC₅₀ values, followed by Rucaparib and Olaparib. Veliparib has the least impact on the number of detected Kuramochi cells of the five PARP inhibitors tested. Niraparib and Rucaparib almost eliminate the Kuramochi culture by t=50 h. The results are shown in FIG. 1B.

### Example 2

### Parp Inhibitor Upregulates Surface Phosphatidylserine and has Negligible Impact on CD4/CD8 T Cells

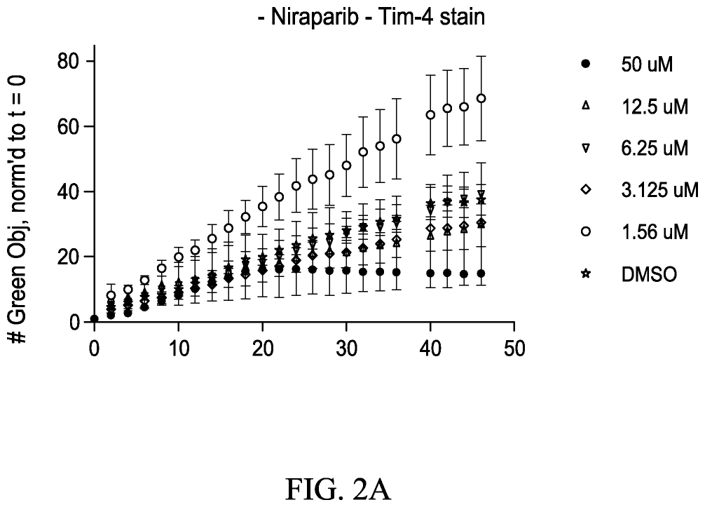

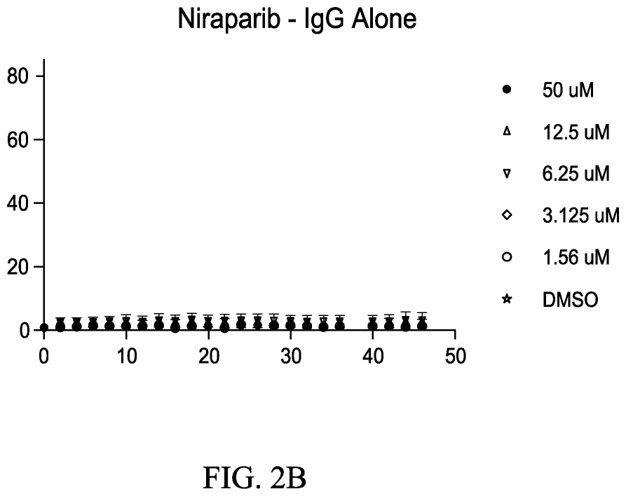

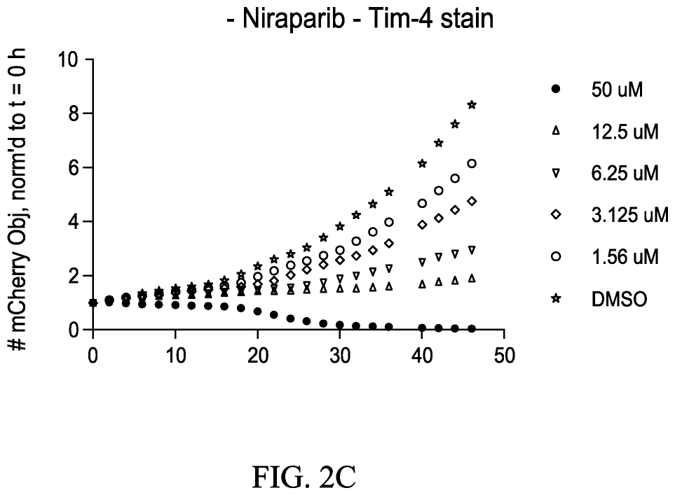

Kuramochi cells expressing a mCherry-NLS plasmid were incubated with various concentrations of Niraparib, a Tim4-Fc chimera, and an anti-mouse IgG2a antibody conjugated to Alexa488. The number of Tim4+Kuramochi cells (i.e., green objects) increases significantly above the IgG only control in response to incubation with even 1.56 μM Niraparib (FIGS. 2A-2B). The number of Kuramochi cells increases in response to 1.56-12.5 μM Niraparib (FIG. 2C), indicating that PARP inhibition induces surface phosphatidylserine without killing Kuramochi cells.

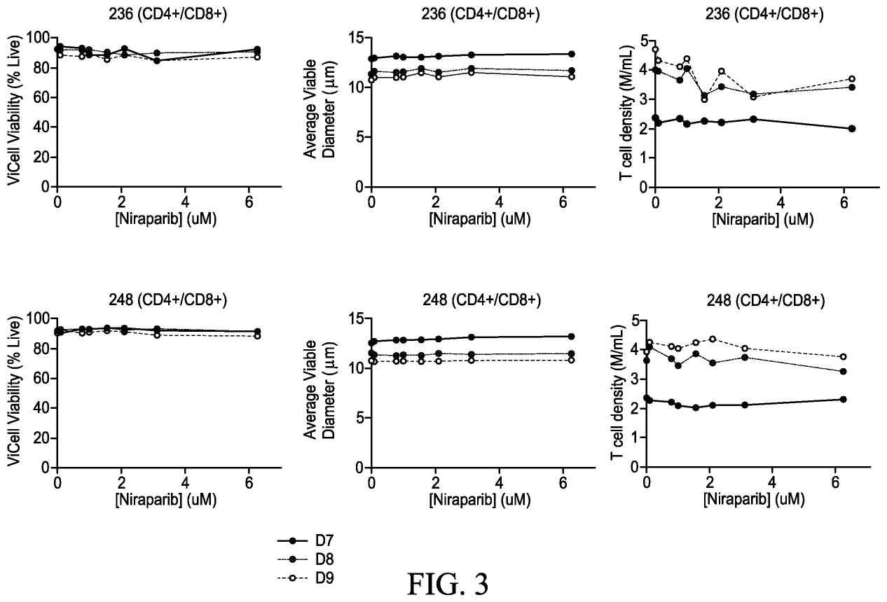

Co-cultures of primary human CD4+ and CD8+ T cells were incubated with various concentrations of Niraparib and T cell viability, diameter, and density were measured using a ViCell cell counter on days 7, 8, and 9 after activation with TransAct, corresponding to days 1, 2, and 3 after incubation with Niraparib. T cell viability, average diameter, and expansion from day 7 to day 9 exhibit negligible changes as a function of Niraparib concentration (FIG. 3).

### Example 3

### Chimeric Tim Receptor/Niraparib Synergistically Kill Kuramochi Cells in Vitro at Different Doses

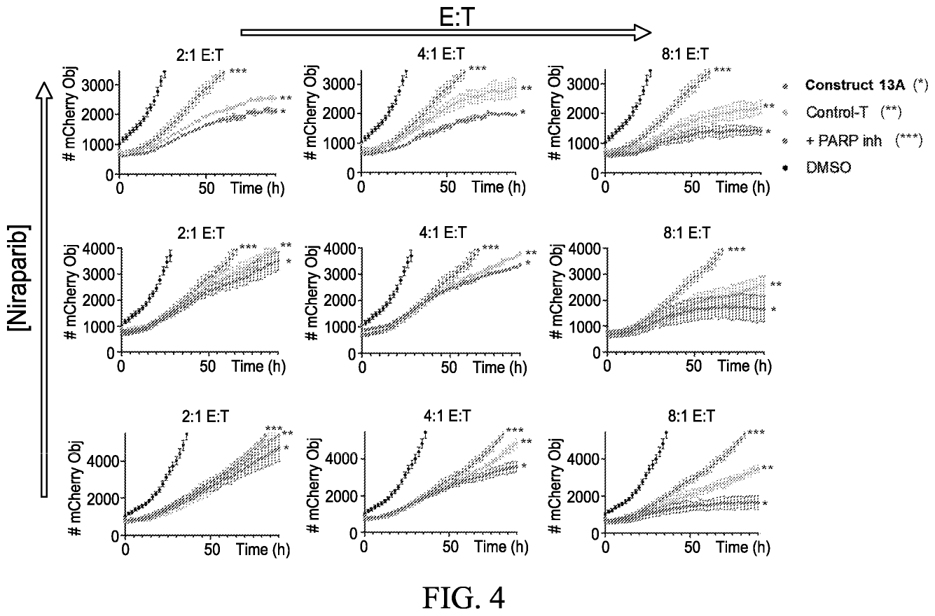

Kuramochi cells expressing a mCherry-NLS plasmid were incubated for 20 hours with 25, 12.5, or 6.25 μM Niraparib. The next day the Niraparib concentration was reduced to 0.52 μM and co-cultures of primary human CD4+ and CD8+ T cells expressing chimeric Tim receptor construct 13A (TIM4 binding domain-CD28 transmembrane domain—CD28 costimulatory domain-CD3ζ signaling domain) (SEQ ID NO 162) were added to the Kuramochi culture. T cell cultures expressing chimeric Tim receptor construct 13A significantly reduce the number of Kuramochi cells in culture below the mock transduction control (control-T), depending on the experimental conditions. The number of Kuramochi cells expressing mCherry-NLS was monitored via IncuCyte. The results are shown in FIG. 4. Combination therapy with chimeric Tim receptor Construct 13A and niraparib synergistically kill Kuramochi cells at different doses.

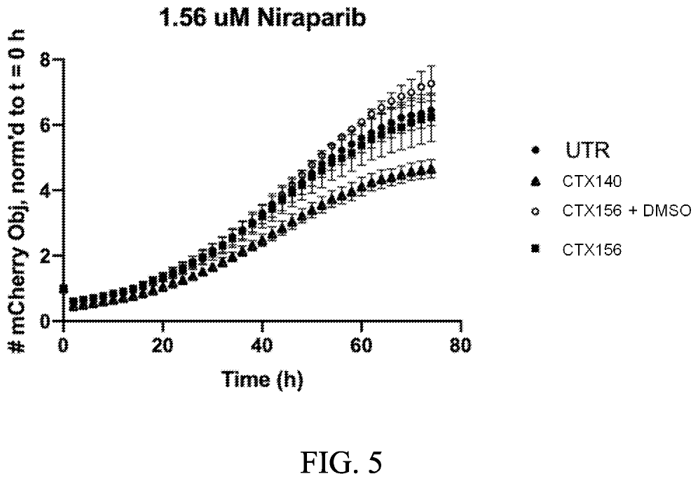

Kuramochi cells expressing a mCherry-NLS plasmid were incubated for ˜20 hours with 1.56 μM Niraparib. The next day, media were removed from cells and replaced with fresh media with 0 μM Niraparib, and primary human CD4+ and CD8+ T cells expressing chimeric Tim receptor CTX140 (Tim4 binding domain-CD28 hinge-CD28 transmembrane-CD28 costimulatory domain-DAP12)(SEQ ID NO: 195) or CTX156 (tEGFR control) were added to the Kuramochi culture. The number of Kuramochi cells expressing mCherry-NLS was monitored via IncuCyte. The results are shown in FIG. 5. T cell cultures expressing chimeric Tim receptor construct CTX140 significantly reduce the number of Kuramochi cells in culture below the mock transduction control.

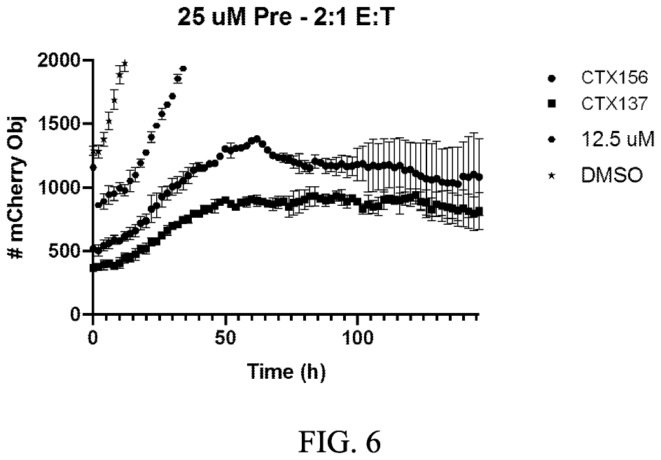

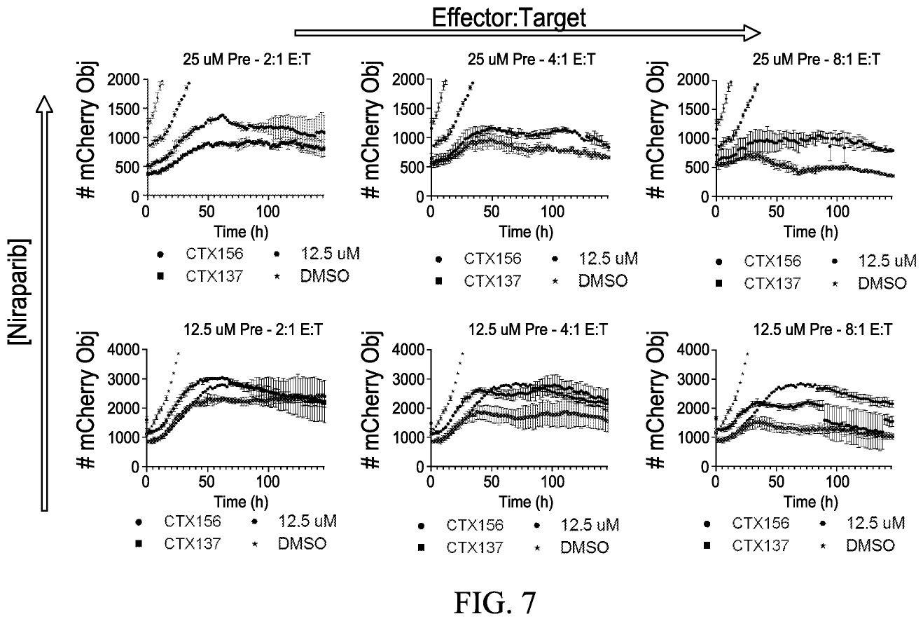

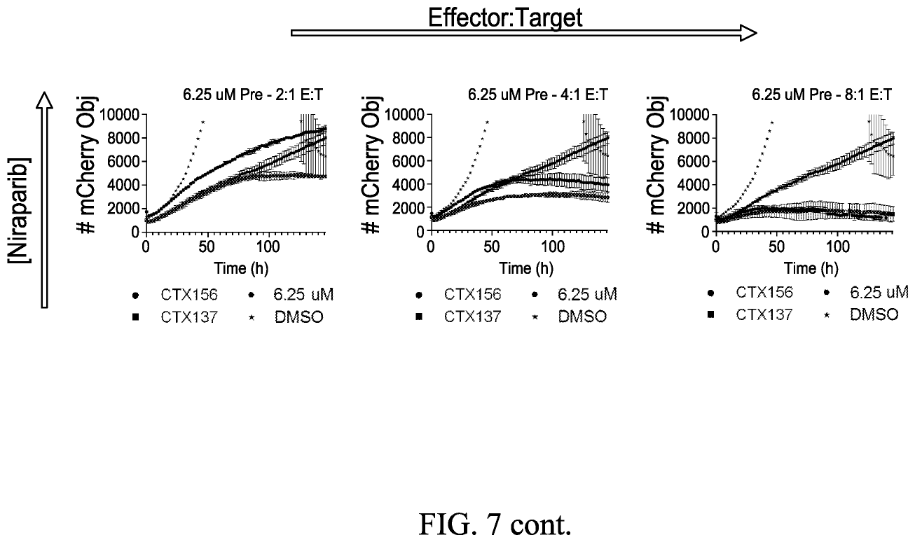

21500 Kuramochi ovarian cancer cells expressing an mCherry-NLS plasmid were plated on day 0.25, 12.5, or 6.25 μM Niraparib were added to cell culture ˜4 hours later. ˜20 hours after initial Niraparib dose, primary human CD4+ and CD8+ T cells expressing chimeric Tim receptor CTX137 (Tim4 binding domain-CD28 transmembrane-CD28 costimulatory domain-DAP12) (SEQ ID NO: 197) or CTX156 (tEGFR control) and maintenance dose of 0.52 μM Niraparib were added. The results for 25 μM Niraparib pre-treatment are shown in FIG. 6. The results for 25, 12.5, or 6.25 μM Niraparib pre-treatment with varying effector:target (E:T) ratios are shown in FIG. 7. T cell cultures expressing chimeric Tim receptor construct CTX137 significantly reduce the number of Kuramochi cells in culture below the mock transduction control.

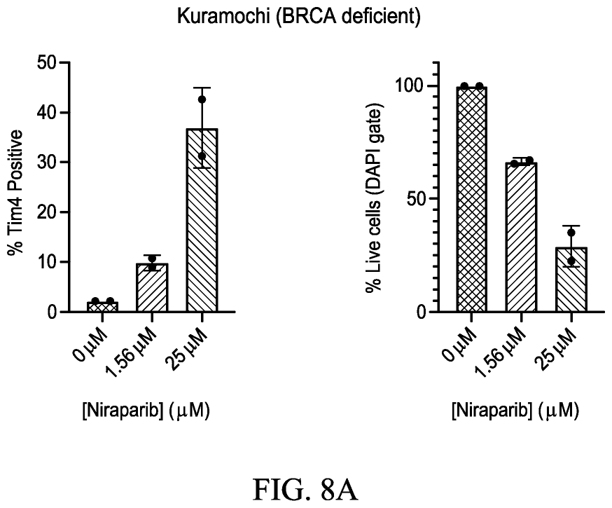

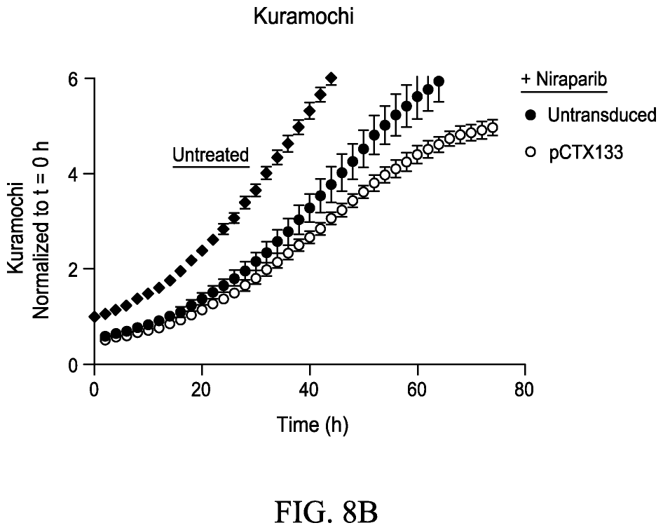

To induce Ptd-Ser exposure, BRCA-2 mutated Kuramochi cell line was treated with therapeutic doses of the PARP inhibitor Niraparib. Brief exposure to Niraparib elicits changes in membrane phospholipid symmetry, in a dose dependent manner, and is effective in inhibiting growth of Kuramochi cells (FIG. 8A). The addition of chimeric Tim4 receptor pCTX133 (Tim4-TLR2-CD3z), at low effector:target ratios (1:1), enhanced the potency of Niraparib in vitro compared to transduced-controls, demonstrating the ability of pCTX133 to elicit direct cytotoxic effects on target cells (FIG. 8B).

### Example 4

### Chimeric Tim Receptor/Niraparib Synergistically Kill A2780 Cells

T cell transduction and culturing: CD4 and CD8 T cells were isolated from frozen healthy donor PBMCs using Miltenyi CD4+ and CD8+ isolation kits. CD4 and CD8 T cells were then mixed in a 1:1 ratio and incubated overnight in a flat bottom 24 well plate in OpTmizer medium supplemented with cell serum replacement, IL-Glutamine, GlutaMAX, and IL-2, IL-7, and IL-15 cytokines, and TransAct diluted 1:50. 16-24 hours past activation with TransAct the T cells were transduced with the appropriate virus at an MOI=10 with 8 μg/mL polybrene and 1:50 TransAct in a flat bottom 96 well plate. ˜24 hours after transduction the T cells were transferred to a 24 well GRex plate in OpTmizer medium supplemented with cell serum replacement, L-Glutamine, GlutaMAX, and IL-2, IL-7, and IL-15 cytokines. T cells were counted and supplemented with fresh medium approximately every two days thereafter.

Cell line Engineering: A2780 (human ovarian cancer cell line) and Kuramochi (BRCA deficient human ovarian cancer cell line) cells were transduced with lentiviral vectors encoding a mCherry-Nuclear localization sequence (NLS) gene and then sorted according to mCherry expression. A2780-mCherry-NLS cell lines were clonally sorted, expanded, and banked, whereas Kuramochi-mCherry-NLS cells were expanded and banked in bulk. Cell lines were thawed and kept in culture up to ˜passage 25 in RPMI 1640 supplemented with 10% FBS.

Co-culture assays: On Day 0 2,500 A2780-mCh-NLS or Kuramochi-mCh-NLS cells were plated in a NUNC Edge 96-well plate and Niraparib (Free Base) was added ˜4 hours later at the appropriate concentration, all in RPMI 1640+10% FBS. Approximately 20 hours later the medium was removed and replaced with the appropriate concentration of Niraparib (Free Base) and the appropriate number of T cells, all in OpTmizer medium supplemented with cell serum replacement, L-Glutamine, GlutaMAX, and IL-2, IL-7, and IL-15 cytokines. When A2780-mCh-NLS cells were used, the supernatant was centrifuged and resuspended in T cell medium to preserve any suspension component of A2780 cells. Plates were immediately transferred to IncuCyte when appropriate.

Flow cytometry measurement of surface PS: A2780-mCh-NLS cells were treated with 1.56 or 25 LIM Niraparib or with equivalent volume of DMSO (control). 48 hours later samples were trypsinized and stained using a Tim4-Fc followed by a fluorescently-labeled secondary antibody to the Tim4-Fc. Samples were immediately analyzed via Flow cytometry.

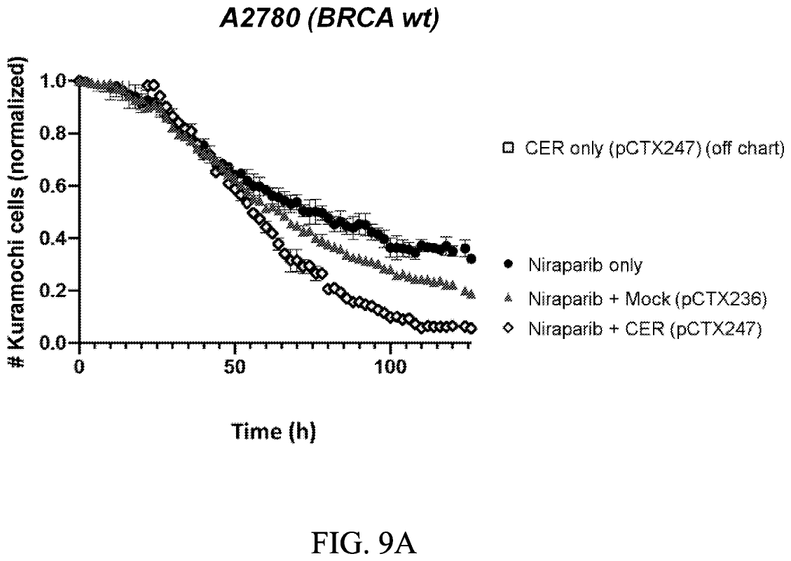

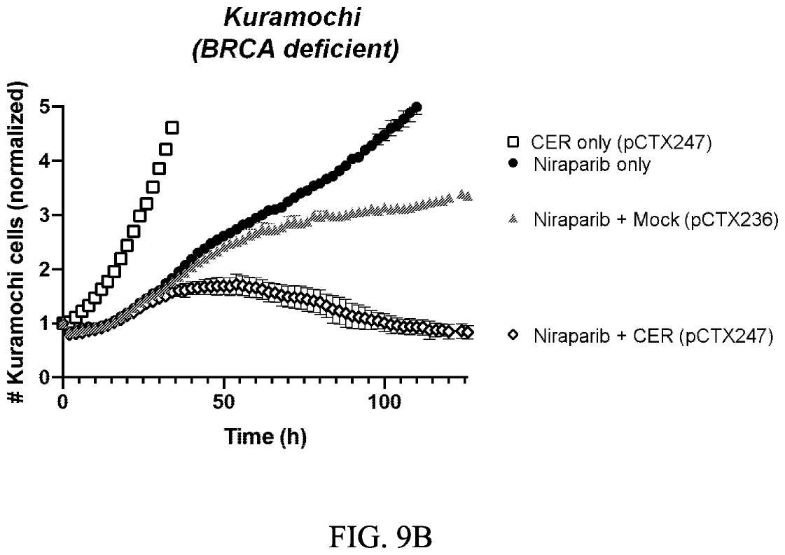

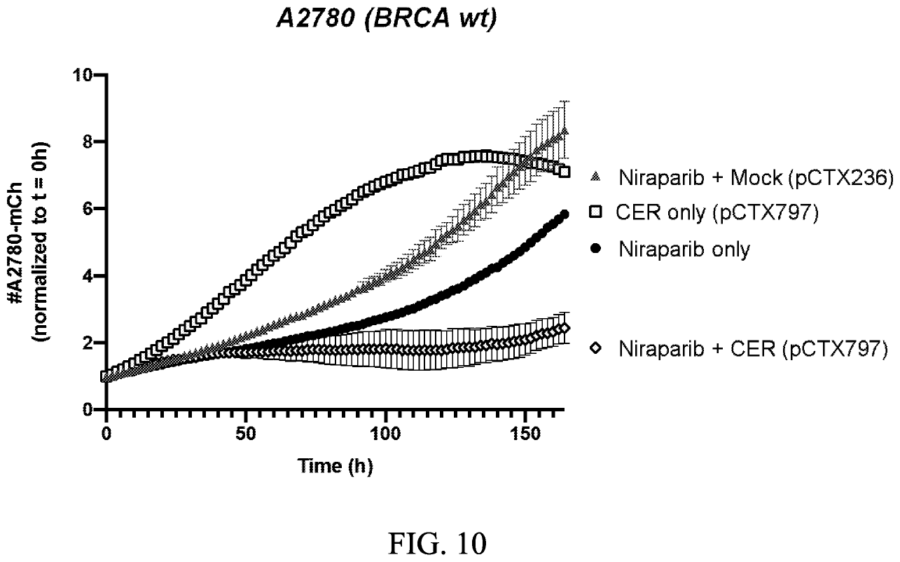

As shown in FIGS. 9A-9B, T cells modified with chimeric Tim4 receptor pCTX247 (Tim4 binding domain-CD28 transmembrane—CD28 signaling domain—CD3ζ signaling domain; SEQ ID NO:257) demonstrate in vitro anti-tumor activity in two ovarian cancer cell lines. As shown in FIG. 10, T cells modified with chimeric Tim4 receptor pCTX797 (Tim4 binding domain-CD28 transmembrane—CD28 signaling domain—CD3ζ signaling domain; SEQ ID NO:258) demonstrate in vitro anti-tumor activity in A2780 ovarian cancer model.

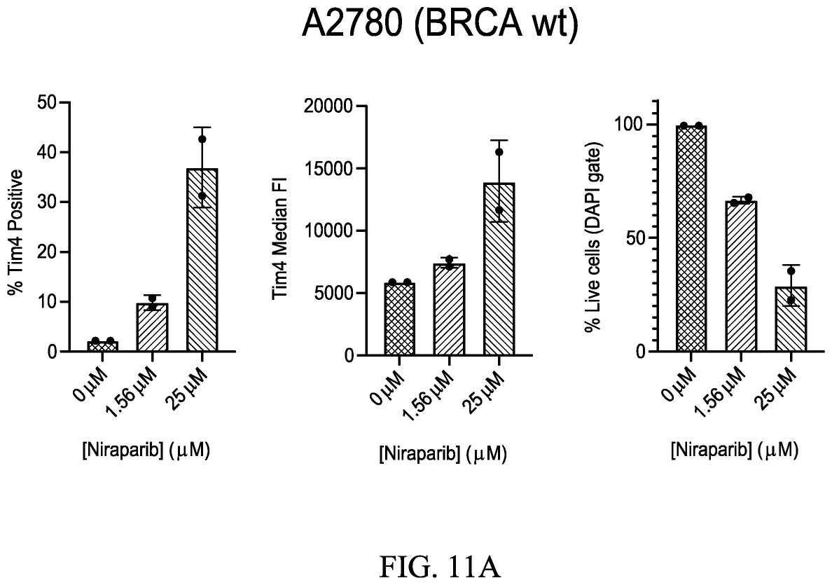

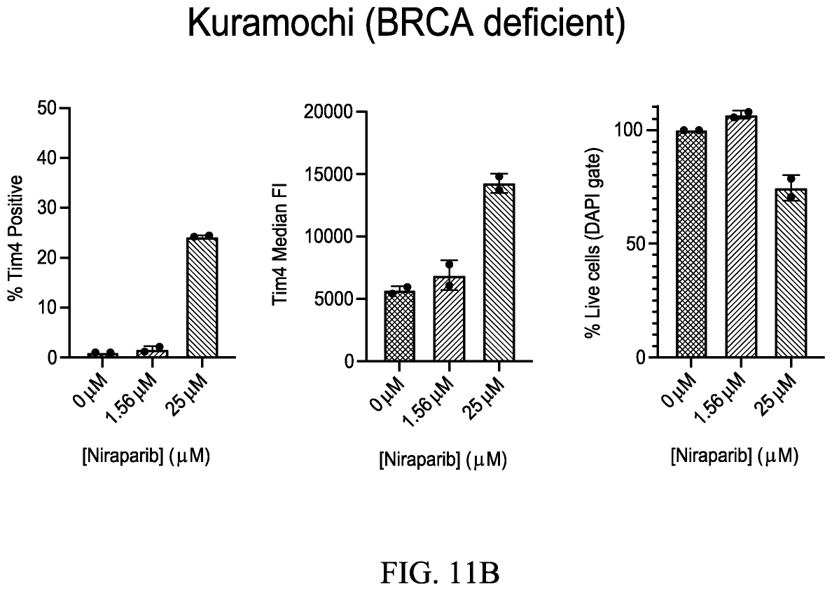

As shown in FIG. 11A-11B, Phosphatidyl serine is present on the cell surface in response to Niraparib in two ovarian cancer models.

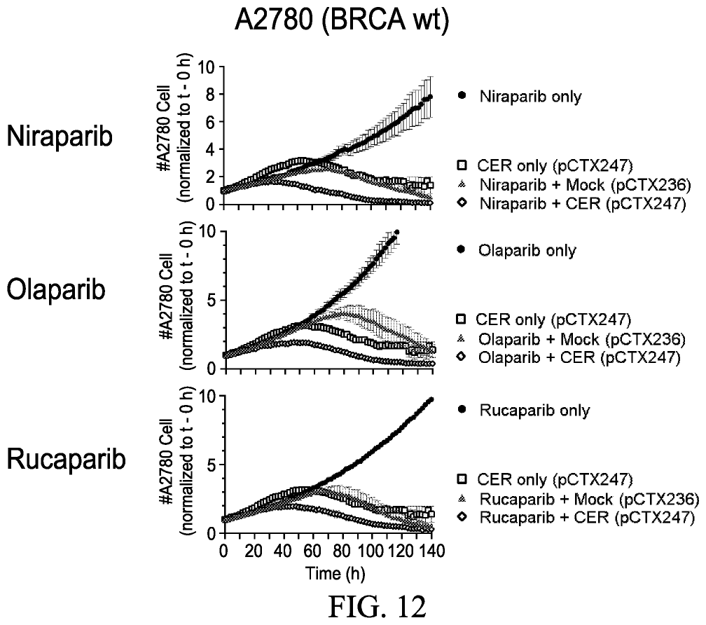

As shown in FIG. 12, chimeric Tim4 receptor pCTX797 (Tim4 binding domain-CD28 transmembrane—CD28 signaling domain—CD3ζ signaling domain; SEQ ID NO:258) exhibits synergy with additional PARP inhibitors (rucarparib, olaparib, and niraparib) compared to truncated EGFR (EGFRt) control (pCTX236).

The various embodiments described above can be combined to provide further embodiments. All of the U.S. patents, U.S. patent application publications, US. patent applications, foreign patents, foreign patent applications and non-patent publications referred to in this specification and/or listed in the Application Data Sheet including but not limited to, U.S. Provisional Patent Application No. 63/066,150, filed on Aug. 14, 2020, U.S. Provisional Patent Application No. 63/087,049, filed on Oct. 2, 2020, and U.S. Provisional Patent Application No. 63/226,712 filed on Jul. 28, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating a cancer, wherein the cancer is breast cancer, ovarian cancer, colorectal cancer, fallopian cancer, peritoneal cancer, prostate cancer, lung cancer, or melanoma, in a subject in need thereof, the method comprising administering to the subject an effective amount of an engineered T cell comprising:
a chimeric engulfment receptor comprising a single chain chimeric protein, the single chain chimeric protein comprising:
(a) an extracellular domain comprising a binding domain comprising:
(i) a Tim4 IgV domain; and
(ii) a Tim4 mucin domain;
(b) an intracellular signaling domain, wherein the intracellular signaling domain comprises a primary intracellular signaling domain, wherein the primary signaling domain comprises a CD28 signaling domain or a TLR2 signaling domain and a secondary intracellular signaling domain, wherein the secondary signaling domain comprises a CD3ζ signaling domain or a DAP12 signaling domain; and
(c) a transmembrane domain positioned between and connecting the extracellular domain and the intracellular signaling domain; and
a Poly (ADP-ribose) polymerase (PARP) inhibitor).

2. The method of claim 1, wherein the breast cancer is triple negative breast cancer.

3. The method of claim 1, wherein the ovarian cancer is advanced ovarian cancer.

4. The method of claim 1, wherein the prostate cancer is advanced prostate cancer.

5. The method of claim 1, wherein the lung cancer is non-small cell lung cancer.

6. The method of claim 1, wherein the cancer is a BReast CAncer gene (BRCA) mutated cancer.

7. (canceled)

8. The method of claim 1, wherein the PARP inhibitor is talazoparib, niraparib, rucaparib, olaparib, veliparib, CEP 9722, E7016, AG014699, MK4827, BMIN-673, Pamiparib, or a combination thereof.

9.-19. (canceled)

20. The method of any one of claim 1, further comprising administering an additional therapeutic agent comprising radiation, cellular immunotherapy, chimeric antigen receptor, T cell receptor, antibody, immune checkpoint molecule inhibitor, chemotherapy, hormone therapy, peptide, antibiotic, anti-viral agent, anti-fungal agent, anti-inflammatory agent, UV light therapy, electric pulse therapy, high intensity focused ultrasound therapy, oncolytic virus therapy, a small molecule therapy, or a combination thereof.

21. (canceled)

22. (canceled)

23. The method of claim 20, wherein the additional therapeutic agent comprises an angiogenesis inhibitor (e.g., a VEGF pathway inhibitor), tyrosine kinase inhibitor (e.g., an EGF pathway inhibitor), receptor tyrosine kinase inhibitor, growth factor inhibitor, GTPase inhibitor, serine/threonine kinase inhibitor, transcription factor inhibitor, B-Raf inhibitor, RAF inhibitor, MEK inhibitor, mTOR inhibitor, EGFR inhibitor, ALK inhibitor, ROS1 inhibitor, BCL-2 inhibitor, PI3K inhibitor, VEGFR inhibitor, BCR-ABL inhibitor, MET inhibitor, MYC inhibitor, ABL inhibitor, HER2 inhibitor, BTK inhibitor, H-RAS inhibitor, K-RAS inhibitor, PDGFR inhibitor, TRK inhibitor, c-KIT inhibitor, c-MET inhibitor, CDK4/6 inhibitor, FAK inhibitor, FGFR inhibitor, FLT3 inhibitor, IDH1 inhibitor, IDH2 inhibitor, PDGFRA inhibitor, or RET inhibitor.

24.-31. (canceled)

32. The method of claim 1, wherein the Tim4 IgV domain comprises the amino acid sequence set forth in SEQ ID NO:34, and/or Tim4 mucin domain comprises the amino acid sequence set forth in SEQ ID NO:35.

33.-35. (canceled)

36. The method of any claim 1, wherein the transmembrane domain comprises a Tim4 transmembrane domain or CD28 transmembrane domain.

37. The method of claim 36, wherein the Tim4 transmembrane domain comprises the amino acid sequence set forth in SEQ ID NO:6 or 23, or the CD28 transmembrane domain comprises the amino acid sequence of SEQ ID NO:7.

38. The method of claim 1, wherein the chimeric Tim receptor further comprises an extracellular spacer domain.

39.-49. (canceled)

50. The method of claim 1, wherein the CD28 signaling domain comprises the amino acid sequence set forth in SEQ ID NO:4 or 26, the DAP12 signaling domain comprises the amino acid sequence set forth in SEQ ID NO:9, the CD3ζ signaling domain comprises the amino acid sequence set forth in SEQ ID NO:5, or the TLR2 signaling domain comprises the amino acid sequence set forth in SEQ ID NO:222.

51.-64. (canceled)

65. The method of claim 1, wherein the chimeric engulfment receptor comprises:
(i) the amino acid sequence of SEQ ID NO:195 or amino acids 25-473 of SEQ ID NO:195;
(ii) the amino acid sequence of SEQ ID NO:196 or amino acids 25-446 of SEQ ID NO:196;
(iii) the amino acid sequence of SEQ ID NO:197 or amino acids 25-434 of SEQ ID NO:197; or
(iv) the amino acid sequence of SEQ ID NO:198 or amino acids 25-428 of SEQ ID NO:198.

66.-75. (canceled)

76. The method of claim 1, wherein the chimeric receptor comprises:
(a) an extracellular domain comprising a binding domain comprising: (i) a Tim4 IgV domain and a Tim4 mucin domain;
(b) an intracellular signaling domain, wherein the intracellular signaling domain comprises a primary intracellular signaling domain comprising CD3ζ signaling domain; a secondary intracellular signaling domain comprising a CD28 signaling domain; and a tertiary intracellular signaling domain comprising a TLR2 signaling domain; and
(c) a transmembrane domain positioned between and connecting the extracellular domain and the intracellular signaling domain.

77.-79. (canceled)

80. The method of claim 1, wherein the chimeric engulfment receptor comprises the amino acid sequence of set forth in any of SEQ ID NOS: 162, 195-198, 71, 227, 228, 229, 232, 233, 238-240, 243, and 244.

81.-85. (canceled)

86. The method of claim 1, wherein the engineered T cell is a CD4+ T cell, a CD8+ T cell, or a CD4+/CD8+ T cell.

87. The method of claim 1, wherein the T cell is a human cell.

88. The method of claim 1, wherein the engineered T cell is administered as a composition further comprising a pharmaceutically acceptable excipient.
